(19) United States
(12) Patent Application Publication
(10) Pub. No.: US 2003/0114445 A1
(43) Pub. Date: Jun. 19, 2003

(54) N3-substituted 6-anilinopyrimidines and methods to treat-Gram-positive bacterial and mycoplasmal infections

(76) Inventors: Chengxin Zhi, Worcester, MA (US); Zheng-Yu Long, Worcester, MA (US); George E. Wright, Worcester, MA (US); Neal C. Brown, Greenfield, NH (US); Serge Lamothe, Boisbriand (CA); Irina Motorina, Laval (CA)

Correspondence Address: FISH & RICHARDSON PC, 225 FRANKLIN ST, BOSTON, MA, 02110, US

(21) Appl. No.: 10/174,131

(22) Filed: Jun. 17, 2002

Related U.S. Application Data
(60) Provisional application No. 60/298,351, filed on Jun. 15, 2001.
(60) Provisional application No. 60/348,477, filed on Jan. 14, 2002.

(51) Int. Cl. A61K 31/541; A61K 31/5377; A61K 31/513; C07D 417/02; C 07D 4 3/02; C07D 413/02

(52) U.S. Cl. 514/227.8; 514/252.14; 514/269; 514/235.8; 544/60; 544/295; 544/311; 544/123

(57) ABSTRACT

Compounds useful for treating Gram-positive bacterial and mycoplasmal infections are disclosed. The compounds have the general formulae shown below.

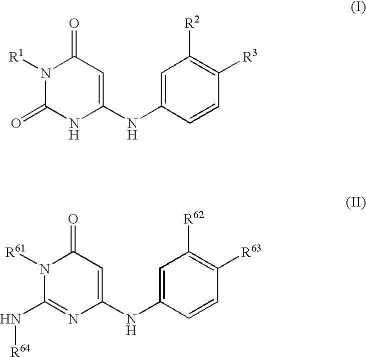

### CROSS-REFERENCE TO RELATED APPLICATIONS

This application claims benefit of priority from U.S. Provisional Patent Application Nos. 60/298,351, filed on Jun. 15, 2001, and No. 60/348,477, filed on Jan. 14, 2002, both of which are incorporated herein by reference in their entirety.

### STATEMENT AS TO FEDERALLY SPONSORED RESEARCH

The invention described herein was supported in part by STTR grant number AI41260-1 and SBIR grant number AI41260-O₂-03 from the National Institutes of Health. The government thus has certain rights in the invention.

### TECHNICAL FIELD

This invention relates to anti-bacterial and anti-mycoplasmal compounds, and more particularly to N3-substituted 6-anilinopyrimidine compounds.

### BACKGROUND

Gram-positive pathogens pose a serious threat to public health. Two of these pathogens, Staphylococcus aureus and Enterococcus fecalis/fecium, are primarily nosocomial (hospital-acquired) pathogens; together, they presently account for the majority of nosocomial diseases. A third organism, Streptococcus pneumoniae, is a community-acquired pathogen. Mycoplasma also pose threats to public health.

Staphylococcus aureus is currently the most frequent cause of nosocomial bacteremia and skin/wound infection and the second most frequent cause of nosocomial lower respiratory infection. Enterococcus fecalis/fecium ranks third behind Staphylococcus aureus and Escherichia coli as a cause of nosocomial septicemia, endocarditis, and infections of wounds and the urinary tract. Streptococcus pneumoniae causes several serious and potentially life-threatening diseases. In the United States it is estimated that Streptococcus pneumoniae accounts annually for 6,000 cases of pneumococcal meningitis, a half million cases of pneumonia, 55,000 cases of bacteremia, and 6 million cases of otitis media. Annual mortality from Streptococcus pneumoniae-induced disease is estimated to be 40,000 in the United States and 3-5 million globally.

There is a rapidly growing global crisis in the clinical management of life-threatening infectious disease caused by multi-antibiotic-resistant strains of the Gram-positive pathogens.

Streptococcus, Enterococcus, and Staphylococcus. New Gram-positive specific antibiotic compounds which can selectively hit these targets must be researched and developed as part of the effort to successfully meet this crisis.

### SUMMARY

The invention is based on the discovery that the N3-substituted-6-anilinopyrimidine compounds disclosed herein have potent anti-bacterial and anti-mycoplasmal properties. Some of these compounds can form salts that are very soluble in water; the compounds can therefore be administered in water or in physiological saline. The compounds can be administered to prevent or to treat Gram-positive bacterial or mycoplasmal infections in eukaryotic cell cultures, animals, or humans.

In one aspect, the invention features compounds having the formula shown below:

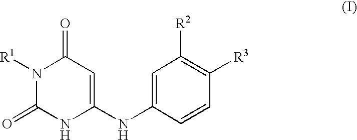

wherein R¹ is (CH₂)ₘ-{(A)ₙ-(CH₂)ₚ}_{q}—B,

in which each A is, independently, CH₂, CH═CH, C≡C, CO, O, S, NR⁸, where R⁸ is H or C₁₋₆ alkyl, CHR¹⁰, where R¹⁰ is OH or C₁₋₆ alkyl, CH(CR¹²R¹³)ᵣCH, where each of R¹² and R¹³ is, independently, H, halogen, or C₁₋₆ alkyl, OCO, CONR¹⁴, NR¹⁵CO, where each of R¹⁴ and R¹⁵ is, independently, H or C₁₋₆ alkyl, SO₂NH, or NHSO₂;

B is H, halogen, substituted or unsubstituted C₁₋₁₀ alkyl, C₃₋₈ cycloalkyl, C₅₋₁₅ heteroaryl, NH₂, CN, OR¹⁶, SR¹⁸, COR¹⁹, OCOR²⁰, NR²¹(CO)R²², NR²³R²⁴, NR²⁵ (CO)NHR²⁶, CN, CH(CO₂R²⁸)₂, CO₂R³⁰, NHSO₂R³², CONR³⁴R³⁶, or CH₂COR³⁸, in which each of R¹⁶-R³⁸ is, independently, H, substituted or unsubstituted C₁₋₆ alkyl, substituted or unsubstituted C₁₋₆ cycloalkyl, substituted or unsubstituted C₆₋₁₂ aryl, substituted or unsubstituted C₇₋₂₀ arylalkyl, substituted or unsubstituted C₇₋₂₀ alkylaryl, substituted or unsubstituted C₄₋₁₀ heteroaryl, C₁₋₃ acyl, or C₁₋₆ sulfonyl, or B is a substituted or unsubstituted 5-8 membered non-aromatic heterocycle;

wherein m is 1-4, n is 0 or 1, p is 0-4, q is 0-4, and r is 1-4; and

wherein each of R² and R³ is, independently, C₁₋₆ alkyl, C₂₋₆ alkenyl, or halogen, or R² and R³ together are C₃₋₅ alkylene;

provided that R¹ is not unsubstituted alkyl, hydroxy-substituted alkyl, alkoxy-substituted alkyl, carboxy-substituted alkyl, amino-substituted alkyl, (substituted-amino)-substituted alkyl, amido-substituted alkyl, carbamate-substituted alkyl, halogen-substituted alkyl, thio-substituted alkyl, azido-substituted alkyl, dithio-substituted alkyl, sulfonyl-substituted alkyl, or alkenyl;

further provided that when A is OCO, then B is not CO₂H, NH₂, or CH(CO₂R)₂;

further provided that when A is C═C, then B is not H or alkyl;

further provided that when A is NH, then B is not CO₂R;

or a pharmaceutically acceptable salt thereof.

In another aspect, the invention features compounds having the formula shown below:

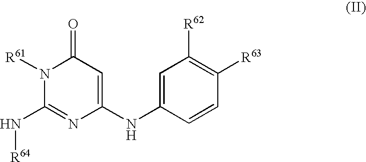

wherein R⁶¹ is (CH₂)ₜ-{(E)ₙ-(CH₂)ᵥ}_{w}-J,

in which each E is, independently, CH₂, CH═CH, C≡C, CO, O, S, NR⁷⁰, where R⁷⁰ is H or C₁₋₆ alkyl, CHR⁷¹, where R⁷¹ is OH or C₁₋₆ alkyl, CH(CR⁷²R⁷³)CH, where each of R⁷² and R⁷³ is, independently, H, halogen, or C₁₋₆ alkyl, OCO, CONR⁷⁴, NR⁷⁵CO, where each of R⁷⁴ and R⁷⁵ is, independently, H or C₁₋₆ alkyl, SO₂NH, or NHSO₂;

J is H, halogen, substituted or unsubstituted C₁₋₁₀ alkyl, C₃₋₈ cycloalkyl, C₅₋₁₀ heteroaryl, NH₂, CN, OR⁷⁶, SR⁷⁸, COR⁷⁹ OCOR⁸⁰, NR⁸¹(CO)R⁸², NR⁸³ R⁸⁴, NR⁸⁵(CO)NHR⁸⁶, CN, CH(CO₂R⁸⁸)₂, CO₂R⁹⁰, NHSO₂R⁹⁰, CONR⁹⁴R⁹⁶, or CH₂COR⁹⁸, in which each of R⁷⁶-R⁹⁸ is, independently, H, substituted or unsubstituted C₁₋₆ alkyl, substituted or unsubstituted C₁₋₆ cycloalkyl, substituted or unsubstituted C₆₋₁₂ aryl, substituted or unsubstituted C₇₋₂₀ arylalkyl, substituted or unsubstituted C₇₋₂₀ alkylaryl, substituted or unsubstituted C₄₋₁₀ heteroaryl, C₁₋₃ acyl, or C₁₋₆ sulfonyl, or B is a substituted or unsubstituted 5-8 membered non-aromatic heterocycle;

wherein R⁶⁴ is H, substituted or unsubstituted C₁₋₆ alkyl, or substituted or unsubstituted C₆₋₁₂ aryl;

wherein each of R⁶² and R⁶³ is, independently, C₁₋₆ alkyl, C₂₋₆ alkenyl, or halogen, or R⁶² and R⁶³ together are C₃₋₅ alkylene; and

wherein t is 1-4, u is 0 or 1, v is 0-4, w is 0-4, and x is 1-4;

provided that R⁶¹ is not unsubstituted alkyl, hydroxy-substituted alkyl, alkoxy-substituted alkyl, carboxy-substituted alkyl, amino-substituted alkyl, amido-substituted alkyl, carbamate-substituted alkyl, halogen-substituted alkyl, thio-substituted alkyl, azido-substituted alkyl, dithio-substituted alkyl, or sulfonyl-substituted alkyl;

further provided that when E is OCO, then J is not CO₂H, NH₂, or CH(CO₂R)₂;

further provided that when E is C═C, then J is not H or alkyl;

further provided that when E is NH, then J is not CO₂R;

or a pharmaceutically acceptable salt thereof.

This invention also provides pharmaceutical compositions including a compound as described above, methods for inhibiting bacterial growth, and methods for therapeutically or prophylactically treating a subject (e.g., an animal or a human) with a bacterial infection. The compounds described herein inhibit Gram-positive bacterial and mycoplasmal DNA polymerase III; the compounds thus inhibit the growth of bacteria and mycoplasmata. The invention further features pharmaceutical compositions containing these compounds; methods for inhibiting the growth of Gram-positive bacteria and/or mycoplasma using these compounds; and methods for treating subjects (e.g., animals or humans) with, or susceptible to, Gram-positive bacterial or mycoplasmal infections.

As used herein, “inhibiting” means reducing the cellular growth rate by at least 80%. In certain embodiments, the growth can be inhibited by 90%, 95%, or even 99% or more. The degree of inhibition can be ascertained by an in vitro growth assay, e.g., by a standard liquid culture technique. Compounds showing inhibition of colony formation at suitable MICs (minimal inhibitory concentrations), e.g., <100 μg/ml, are useful for further examination as therapeutic agents. Other standards of inhibition testing can also be used. In the context of inhibiting bacterial or mycoplasmal growth, by “effective amount” of a compound is meant an amount which, when administered in vivo or in vitro, will achieve the above-stated levels of inhibition.

The method for treating a subject (e.g., a human) with a Gram-positive bacterial or mycoplasmal infection involves administering to the subject a therapeutically effective amount of a compound of the invention. By “therapeutically effective amount” is meant an amount which, when administered to a subject in need, will alleviate at least one of the symptoms of a bacterial or mycoplasmal infection. In the context of prophylaxis, a “therapeutically effective amount” is an amount which, when administered to a subject susceptible to bacterial or mycoplasmal infection, will help inhibit or reduce the likelihood of such an infection.

“A subject susceptible to a Gram-positive bacterial infection” is meant a subject (e.g., a human or an animal) that is at increased risk, relative to the general population, of contracting a Gram-positive bacterial infection. Examples of such subjects include those that have recently undergone a surgical procedure, or immunocompromised humans, e.g., those with AIDS (acquired immunodeficiency syndrome). Such animals or humans can be identified using methods known to one of ordinary skill in the art.

By “substituted” is meant that one or more hydrogen atoms of a compound or portion of a compound are replaced by substituents, including, but not limited to, C₁₋₄ alkyl, C₁₋₆ cycloalkyl, hydroxyl, C₁₋₄ alkoxyl, amino, carboxyl, halogen, cyano, azido, C₆₋₁₂ aryl, C₇₋₂₀ arylalkyl, C₄₋₆ heteroaryl, (CO)—C₁₋₆ alkyl, (CO)—C₁₋₆ aryl, (SO₂)—C₁₋₆ alkyl, (SO₃)—C₁₋₆ alkyl, (SO₂)—C₆₋₁₂ aryl, (SO₃)—C₆₋₁₂ aryl, (SO₂)—C₄₋₁₂ heteroaryl, (SO₃)—C₄₋₁₂ heteroaryl. The substituents can in turn be substituted with functional groups, including, but not limited to, halogen, trifluoromethyl, hydroxyl, and carboxyl.

“Pharmaceutically acceptable salts” are those salts derived from pharmaceutically acceptable inorganic and organic acids and bases. Examples of suitable acids include hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, formic, benzoic, malonic, naphthalene-2-sulfonic and benzenesulfonic acids. Other acids such as oxalic acid, while not themselves pharmaceutically acceptable, may be useful as intermediates in obtaining the compounds of the invention and their pharmaceutically acceptable acid addition salts. Salts derived from appropriate bases include alkali metal (e.g. sodium), alkaline earth metal (e.g. magnesium), ammonium, and NR⁴⁺ (where R is C₁₋₄ alkyl) salts. Preferred salts include hydrochlorides, hydrobromides, sulfates, mesylates, maleates, and fumarates. References hereinafter to a compound according to the invention includes compounds of the general formulae shown, as well as their pharmaceutically acceptable salts.

The compounds of this invention may contain functional groups that increase the water solubility of the compounds, facilitating their bioavailability, absorption, and distribution in humans and animals, without interfering with their inhibition of growth of Gram-positive bacteria and mycoplasma spp. Alternatively, the compounds form salts that are relatively water-soluble.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying description below. Other features, objects, and advantages of the invention will be apparent from the description, chemical structures, drawings, and claims.

### DESCRIPTION OF DRAWINGS

FIG. 1 is a graph showing plasma concentration time curves for a compound of the invention.

FIG. 2 is a graph showing the effect of several compounds against S. aureus infections in mice.

FIG. 3 is a graph showing the effect of a compound against S. aureus infections in mice.

### DETAILED DESCRIPTION

The invention features compounds, methods, and compositions for treating Gram-positive bacterial infections and mycoplasmal infections. The methods for inhibiting the growth of bacteria or mycoplasma involve administering, in an amount sufficient to be effective for inhibition, compounds of the invention. In addition, the low toxicity of these compounds to mammals and other animals endows this class of agents with the characteristics required of Gram-positive- and mycoplasma-specific therapeutic antimicrobials. The compounds target an essential enzyme in DNA replication that has not previously been a target for any marketed antibiotic; development of drug resistance will thus be minimized. The compounds can be used to circumvent the natural and acquired resistance of pathogenic Gram-positive bacteria and mycoplasma to conventional antimicrobials.

In one aspect, the invention features compounds having the formula shown below:

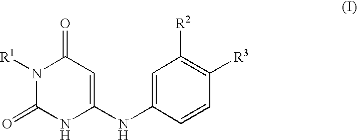

wherein R¹ is (CH₂)ₘ-{(A)ₙ-(CH₂)ₚ}_{q}—B,

in which each A is, independently, CH₂, CH═CH, C≡C, CO, O, S, NR⁸, where R⁸ is H or C₁₋₆ alkyl, CHR¹⁰, where R¹⁰ is OH or C₁₋₆ alkyl, CH(CR¹²R¹³)ᵣCH, where each of R¹² and R¹³ is, independently, H, halogen, or C₁₋₆ alkyl, OCO, CONR¹⁴, NR¹⁵CO, where each of R¹⁴ and R¹⁵ is, independently, H or C₁₋₆ alkyl, SO₂NH, or NHSO₂;

B is H, halogen, substituted or unsubstituted C₁₋₁₀ alkyl, C₃₋₈ cycloalkyl, C₅₋₁₅ heteroaryl, NH₂, CN, OR¹⁶, SR¹⁸, COR¹⁹, OCOR²⁰, NR²¹(CO)R²², NR²³R²⁴, NR²⁵(CO)NHR²⁶, CN, CH(CO₂R²⁸)₂, CO₂R³⁰, NHSO₂R³², CONR³⁴R³⁶, or CH₂COR³⁸, in which each of R¹⁶-R³⁸ is, independently, H, substituted or unsubstituted C₁₋₆ alkyl, substituted or unsubstituted C₁₋₆ cycloalkyl, substituted or unsubstituted C₆₋₁₂ aryl, substituted or unsubstituted C₇₋₂₀ arylalkyl, substituted or unsubstituted C₇₋₂₀ alkylaryl, substituted or unsubstituted C₄₋₁₀ heteroaryl, C₁₋₃ acyl, or C₁₋₆ sulfonyl, or B is a substituted or unsubstituted 5-8 membered non-aromatic heterocycle;

wherein m is 1-4, n is 0 or 1, p is 0-4, q is 0-4, and r is 1-4; and

wherein each of R² and R³ is, independently, C₁₋₆ alkyl, C₂₋₆ alkenyl, or halogen, or R² and R³ together are C₃₋₅ alkylene,

provided that R¹ is not unsubstituted alkyl, hydroxy-substituted alkyl, alkoxy-substituted alkyl, carboxy-substituted alkyl, amino-substituted alkyl, (substituted-amino)-substituted alkyl, amido-substituted alkyl, carbamate-substituted alkyl, halogen-substituted alkyl, thio-substituted alkyl, azido-substituted alkyl, dithio-substituted alkyl, sulfonyl-substituted alkyl, or alkenyl;

further provided that when A is OCO, then B is not CO₂H, NH₂, or CH(CO₂R)₂;

further provided that when A is C═C, then B is not H or alkyl;

further provided that when A is NH, then B is not CO₂R;

or a pharmaceutically acceptable salt thereof.

In one embodiment, n is 1, p is 1-4, q is 1, A is not CH₂, C═C, or C—C, and B is not H, alkyl, or cycloalkyl in the compounds of formula (I) shown above. Furthermore in some embodiments, n is 1, q is 1, A is not CH₂, and B is a 5-8 membered, substituted or unsubstituted non-aromatic heterocycle. In some embodiments, B includes a substituent that is capable of forming a salt with an acid or a base; for example, B can include an amine, a carboxylic acid, a sulfonamide, or an imide. Alternatively, B may include a ring N atom that is capable of forming a salt with an acid or a base; for example, B can include an amine, a sulfonamide, or an imide. In some cases, B is a substituted heterocycle; in some embodiments, B includes a substituent that is capable of forming a salt with an acid or a base, e.g., B can include an amine, a carboxylic acid, a sulfonamide, or an imide. Alternatively, B is a substituted heterocycle including a ring N atom that is capable of forming a salt with an acid or a base, e.g., B can include an amine, a sulfonamide, or an imide.

In some compounds of formula (I), R² is selected from the group consisting of Cl, Br, I, CH₃, CH₂CH₃, and CH═CH₂ (e.g., CH₂CH₃). In some embodiments, R³ is CH₃ and/or

A is O. In some embodiments, R² and R³ together are —CH₂CH₂CH₂—, so the compound of formula (I) is an indanyl.

In some embodiments, compounds of formula (I) are compounds wherein B is

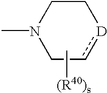

in which D is O, S, NR⁴¹, or C(R⁴²)(R⁴³), in which each of R⁴¹-R⁴³ is, independently, absent, H, C₁₋₆ alkyl, C₆₋₂₀ aryl, C₄₋₉ heteroaryl, C₇₋₁₂ arylalkyl, or COR⁴⁴, in which R⁴⁴ is substituted or unsubstituted C₆₋₁₀ aryl or substituted or unsubstituted C₄₋₆ heteroaryl, OR⁴⁵, in which R⁴⁵ is H, substituted or unsubstituted C₁₋₆ alkyl, substituted or unsubstituted C₁₋₆ cycloalkyl, C₆₋₁₂ aryl, C₇₋₂₀ arylalkyl, C₄₋₆ heteroaryl, C₁₋₃ acyl, or C₁₋₆ sulfonyl; each R⁴⁰ is, independently, substituted or unsubstituted C₁₋₆ alkyl, substituted or unsubstituted C₁₋₆ cycloalkyl, C₆₋₁₂ aryl, C₇₋₂₀ arylalkyl, C₄₋₆ heteroaryl, C₁₋₃ acyl, or C₁₋₆ sulfonyl, OR⁴⁶, CH₂OR⁴⁸, in which each of R⁴⁶ and R⁴⁸ is, independently, H, substituted or unsubstituted C₁₋₆ alkyl, substituted or unsubstituted C₁₋₆ cycloalkyl, C₆₋₁₂ aryl, C₇₋₂₀ arylalkyl, C₄₋₆ heteroaryl, C₁₋₃ acyl, or C₁₋₆ sulfonyl, or CO₂R⁵⁰, where R⁵⁰ is substituted or unsubstituted C₆₋₁₂ aryl, or substituted or unsubstituted C₄₋₆ heteroaryl; and s is 0-2.

In some embodiments, D is O, S, or NR⁴¹, and s is 1 or 2. Alternatively, D may be C(R⁴²)(R⁴³), where at least one of R⁴² and R⁴³ is not H.

This invention also features compounds having the formula shown below:

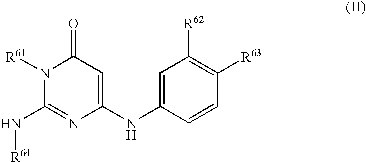

wherein R⁶¹ is (CH₂)ₜ-{(E)ₙ-(CH₂)ᵥ}_{w}-J,

in which each E is, independently, CH₂, CH═CH, C≡C, CO, O, S, NR⁷⁰, where R⁷⁰ is H or C₁₋₆ alkyl, CHR⁷¹, where R⁷¹ is OH or C₁₋₆ alkyl, CH(CR⁷²R⁷³)ₓCH, where each of R⁷² and R⁷³ is, independently, H, halogen, or C₁₋₆ alkyl, OCO, CONR⁷⁴, NR⁷⁵CO, where each of R⁷⁴ and R⁷⁵ is, independently, H or C₁₋₆ alkyl, SO₂NH, or NHSO₂;

J is H, halogen, substituted or unsubstituted C₁₋₁₀ alkyl, C₃₋₈ cycloalkyl, C₅₋₁₀ heteroaryl, NH₂, CN, OR⁷⁶, SR⁷⁸, COR⁷⁹ OCOR⁸⁰, NR⁸¹(CO)R⁸², NR⁸³R⁸⁴, NR⁸⁵(CO)NHR⁸⁶, CN, CH(CO₂R⁸⁸)₂, CO₂R⁹⁰, NHSO₂R⁹², CONR⁹⁴R⁹⁶, or CH₂COR⁹⁸, in which each of R⁷⁶-R⁹⁸ is, independently, H, substituted or unsubstituted C₁₋₆ alkyl, substituted or unsubstituted C₁₋₆ cycloalkyl, substituted or unsubstituted C₆₋₁₂ aryl, substituted or unsubstituted C₇₋₂₀ arylalkyl, substituted or unsubstituted C₇₋₂₀ alkylaryl, substituted or unsubstituted C₄₋₁₀ heteroaryl, C₁₋₃ acyl, or C₁₋₆ sulfonyl, or B is a substituted or unsubstituted 5-8 membered non-aromatic heterocycle;

wherein R⁶⁴ is H, substituted or unsubstituted C₁₋₆ alkyl, or substituted or unsubstituted C₆₋₁₂ aryl;

wherein each of R⁶² and R⁶³ is, independently, C₁₋₆ alkyl, C₂₋₆ alkenyl, or halogen, or R⁶² and R⁶³ together are C₃₋₅ alkylene; and

wherein t is 1-4, u is 0 or 1, v is 0-4, w is 0-4, and x is 1-4;

provided that R⁶¹ is not unsubstituted alkyl, hydroxy-substituted alkyl, alkoxy-substituted alkyl, carboxy-substituted alkyl, amino-substituted alkyl, amido-substituted alkyl, carbamate-substituted alkyl, halogen-substituted alkyl, thio-substituted alkyl, azido-substituted alkyl, dithio-substituted alkyl, or sulfonyl-substituted alkyl;

further provided that when E is OCO, then J is not CO₂H, NH₂, or CH(CO₂R)₂;

further provided that when E is C═C, then J is not H or alkyl;

further provided that when E is NH, then J is not CO₂R;

or a pharmaceutically acceptable salt thereof.

For example, compounds of formula (II) include those compounds where R⁶² is selected from the group consisting of Cl, Br, I, CH₃, CH₂CH₃, or CH═CH₂ (e.g., CH₂CH₃) and those where R⁶³ is CH₃. In some embodiments, or R⁶² and R⁶³ together are —CH₂CH₂CH₂—, and the compound of formula (II) is an indanyl. In some embodiments, R⁶⁴ is selected from the group consisting of CH₃, CH₂CH₃, phenyl, 3-ethyl-4-methylphenyl, 4-hydroxybutyl, 4-aminobutyl, and 4-methoxybutyl.

In other embodiments, in compounds of formula (II), J is

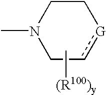

in which G is O, S, NR¹⁰¹, or C(R¹⁰²)(R¹⁰³), in which each of R¹⁰¹-R¹⁰³ is, independently, absent, H, C₁₋₆ alkyl, C₆₋₂₀ aryl, C₄₋₆ heteroaryl, C₇₋₁₂ arylalkyl, or COR¹⁰⁴, in which R¹⁰⁴ is substituted or unsubstituted C₆₋₁₀ aryl or substituted or unsubstituted C₄₋₆ heteroaryl, OR¹⁰⁵, in which R¹⁰⁵ is H, substituted or unsubstituted C₁₋₆ alkyl, substituted or unsubstituted C₁₋₆ cycloalkyl, substituted or unsubstituted C₆₋₁₂ aryl, substituted or unsubstituted C₇₋₂₀ arylalkyl, substituted or unsubstituted C₇₋₂₀ alkylaryl, substituted or unsubstituted C₄₋₁₀ heteroaryl, C₁₋₃ acyl, or C₁₋₆ sulfonyl;

each R¹⁰⁰ is, independently, substituted or unsubstituted C₁₋₆ alkyl, substituted or unsubstituted C₁₋₆ cycloalkyl, substituted or unsubstituted C₆₋₁₂ aryl, substituted or unsubstituted C₇₋₂₀ arylalkyl, substituted or unsubstituted C₇₋₂₀ alkylaryl, substituted or unsubstituted C₄₋₁₀ heteroaryl, C₁₋₃ acyl, or C₁₋₆ sulfonyl, OR¹⁰⁶, CH₂OR¹⁰⁸, in which each of R¹⁰⁶ and R¹⁰⁸ is, independently, H, substituted or unsubstituted C₁₋₆ alkyl, substituted or unsubstituted C₁₋₆ cycloalkyl, substituted or unsubstituted C₆₋₁₂ aryl, substituted or unsubstituted C₇₋₂₀ arylalkyl, substituted or unsubstituted C₇₋₂₀ alkylaryl, substituted or unsubstituted C₄₋₁₀ heteroaryl, C₁₋₃ acyl, or C₁₋₆ sulfonyl, or CO₂R¹¹⁰, where R¹¹⁰ is substituted or unsubstituted C₆₋₁₂ aryl, or substituted or unsubstituted C₄₋₆ heteroaryl;

and y is 0-2.

In the compounds of formula (I) or (II) wherein A is CH(CR¹²R³)ᵣCH, wherein each of R¹² and R¹³ is, independently, H, halogen, or C₁₋₆ alkyl, and r is 1-4, the fragment CH(CR¹²R¹³)ᵣCH includes the possibility of a 3-6 membered ring portion; the ring may be substituted with one or more halogens, or with one or more alkyl chains. For example, the fragment may be one of the fragments shown below:

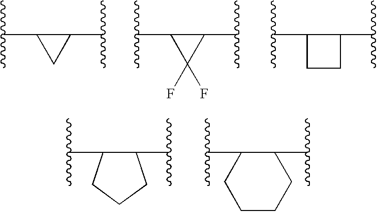

This invention also provides pharmaceutical compositions including, and methods for making and using, the compounds described herein. Such methods include a method of inhibiting growth of Gram-positive bacteria in vitro by contacting the bacteria with an effective amount of a compound according to this invention. Additional methods include a method of treating an animal (e.g., a human) with a Gram-positive bacterial infection by administering to the animal a therapeutically effective amount of a compound according to this invention, and a method of prophylactically treating an animal susceptible to a Gram-positive bacterial infection by administering to the animal a therapeutically effective amount of a compound of this invention. Other methods for using the compounds and compositions according to this invention will be apparent to those of ordinary skill in the art upon reading the present application and are expressly included as a part of this invention.

The compounds described herein have special advantages in the treatment of organisms that have become resistant to currently used therapeutics. For example, these compounds can inhibit the DNA polymerase III enzymes from strains of pathogenic Enterococci, Streptococci, and Staphylococci that are resistant to currently used antibiotics. Inhibition of DNA polymerase III, the enzyme responsible for replication of the genome of the organism, causes inhibition of growth of the organism.

Mechanism of Action

Genome sequence analysis has indicated that organisms such as the Mycoplasmas and Gram-positive eubacteria of the so-called low G:C class, i.e., those with genomes containing a proportion of guanine+cytosine of less than 0.5, contain two types of DNA polymerase III (pol III): pol IIIC, encoded by a polC gene, and pol IIIE, encoded by one or more dnaE genes, (See, Wright, G. and Brown, N. DNA polymerase III: A new target for antibiotic development, Current Opinion in Anti-Infective Investigational Drugs 1:45-48 (1999) and Braithewaite, D. and Ito, J. Compilation, alignment, and phylogenetic relationships of DNA polymerases, Nucl. Acids Res. 21:787-802 (1993)). The compounds described herein are designed to specifically inhibit the pol IIIC enzyme.

Gram-positive pol IIIC is an enzyme that is absolutely required for the replicative synthesis of DNA that accompanies the cyclical duplication of the host chromosome. The compounds described herein mimic purine deoxyribonucleoside-5-triphosphates and physically inhibit DNA polymerase. The mechanism of action of N3-substituted pyrimidines is further described in U.S. Pat. No. 5,516,905. Because the compounds described herein inhibit the DNA polymerase, they are useful for inhibiting the growth of Gram-positive bacteria and mycoplasma, and for treating Gram-positive bacterial and mycoplasmal infections.

Antibacterial and Anti-Mycoplasmal Compounds

The compounds described herein are N3-substituted-6-anilinopyrimidines, for example, N3-substituted-6-anilinouracils and N3-substituted isocytosines. Useful compounds include, but are not limited to: 3-[2-(2-Benzyloxyethoxy)ethyl]-6-(3-ethyl-4-methylanilino)uracil; 3-(4-Ethoxycarbonylbutyl)-6-(3-ethyl-4-methylanilino)uracil; 3-(3-Methoxycarbonyl-2-propenyl)-6-(3-ethyl-4-methylanilino)uracil; 3-(4-Oxopentyl)-6-(3-ethyl-4-methylanilino)uracil; 3-(Ethoxycarbonylmethyl)-6-(3-ethyl-4-methylanilino)uracil; 3-(3-Ethoxycarbonylpropyl)-6-(3-ethyl-4-methylanilino)uracil; 3-[(N,N-Diethylaminocarbonyl)methyl]-6-(3-ethyl-4-methylanilino)uracil; 3-[2-(2-Methoxyethoxy)ethyl]-6-(3-ethyl-4-methylanilino)uracil; 3-(4-Hydroxy-2-butynyl)-6-(3-ethyl-4-methylanilino)uracil; 3-[2-(N,N-Diethylamino)ethyl]-6-(3-ethyl-4-methylanilino)uracil; 3-(2-Oxopropyl)-6-(3-ethyl-4-methylanilino)uracil; 3-[2-(Methanesulfonylamino)ethyl]-6-(3-ethyl-4-methylanilino)uracil; 3-[2-(N-Morpholino)ethyl]-6-(3-ethyl-4-methylanilino)uracil; 3-(8-Hydroxyoctyl)-6-(3-ethyl-4-methylanilino)uracil; 3-(3-Cyanopropyl)-6-(3-ethyl-4-methylanilino)uracil; 3-(4-Cyanobutyl)-6-(3-ethyl-4-methylanilino)uracil; 3-{2-[(2-Hydroxyethoxy)ethoxy)]ethyl}-6-(3-ethyl-4-methylanilino)uracil; 3-(4-Acetoxybutyl)-6-(3-ethyl-4-methylanilino)uracil; 3-(4,5-Dihydroxypentyl)-6-(3-ethyl-4-methylanilino)uracil; 3-[3-(N-Morpholino)propyl]-6-(3-ethyl-4-methylanilino)uracil; (S)-3-(2-Methyl-3-hydroxypropyl)-6-(3-ethyl-4-methylanilino)uracil; 3-[(4-Piperazinyl)butyl]-6-(3-ethyl-4-methylanilino)uracil dihydrochloride; 3-(6-Hydroxyhexyl)-6-(3-ethyl-4-methylanilino)uracil; 3-(4-Aminobutyl)-6-(3-ethyl-4-methylanilino)uracil hydrochloride; 3-[2-(2-Hydroxyethoxy)ethyl]-6-(3-ethyl-4-methylanilino)uracil; 3-(4-Hydroxy-2-butenyl)-6-(3-ethyl-4-methylanilino)uracil; 3-{[2-(Hydroxymethyl)-3,3-difluorocyclopropyl]methyl}-6-(3-ethyl-4-methylanilino)uracil; 3-(4-Iodobutyl)-6-(3-ethyl-4-methylanilino)uracil; 3-[4-(N-morpholino)butyl]-6-(3-ethyl-4-methylanilino)uracil; 3-[(N-Thiomorpholino)butyl]-6-(3-ethyl-4-methylanilino)uracil; 3{[4-(3-Trifluoromethylphenyl-4-chloro-)-4-hydroxypiperidino]butyl}-6-(3-ethyl-4-methylanilino)uracil; 3{[4-(4-Chlorophenyl)-4-hydroxypiperidino]butyl}-6-(3-ethyl-4-methylanilino)uracil; 3-[2-(4-Benzoylpiperazino)ethyl]-6-(3-ethyl-4-methylanilino)uracil; 3-{4-[4-(2-Furoyl)piperazino]butyl}-6-(3-ethyl-4-methylanilino)uracil; 3-[4-(4-Benzylpiperazino)butyl]-6-(3-ethyl-4-methylanilino)uracil; 3-{[4-(3-Hydoxymethyl) morpholino]butyl}-6-(3-ethyl-4-methylanilino)uracil; 3-{[4-(3-Ethoxycarbonylmethyl) morpholino]butyl}-6-(3-ethyl-4-methylanilino)uracil; 3-[4-(cis-2,6-Dimethylmorpholino) butyl]-6-(3-ethyl-4-methylanilino)uracil; 3-{4-[4-(2-Pyrimidinyl)piperazinyl]butyl}-6-(3-ethyl-4-methyylanilino)uracil; 3-{4-[(4-Fluorophenyl)piperazinyl]butyl}-6-(3-ethyl-4-methyylanilino)uracil; 3-(4-Piperazinylbutyl)-6-(3-ethyl-4-methylanilino)uracil dihydrochloride; 3-[4-(N-Morpholinocarbonyloxy)butyl]-6-(3-ethyl-4-methylanilino)uracil; 3-[4-(2-Thienylsulfonylamino)butyl]-6-(3-ethyl-4-methylanilino)uracil; 3-{4-[(Cyclopropylcarbonyl)amino]butyl}-6-(3-ethyl-4-methylanilino)uracil; 3-{4-[(Chloromethylcarbonyl)amino]butyl}-6-(3-ethyl-4-methylanilino)uracil; 3-[4-(2-Chloroacetoxy)butyl]-6-(3-ethyl-4-methylanilino)uracil; 3-(4-Acetoxybutyl)-6-(3-ethyl-4-methylanilino)isocytosine; 3-[2-(N-morpholino)ethyl]-6-(3-ethyl-4-methylanilino) isocytosine; 3-[3-(N-morpholino)propyl]-6-(3-ethyl-4-methylanilino)isocytosine; 3-[4-(N-morpholino)butyl]-6-(3-ethyl-4-methylanilino)isocytosine; 3-[2-(N-morpholino)ethyl]-6-(3-ethyl-4-methylanilino)uracil hydrochloride; 3-[3-(N-morpholino)propyl]-6-(3-ethyl-4-methylanilino)uracil hydrochloride; 3-[4-(N-morpholino)butyl]-6-(3-ethyl-4-methylanilino) uracil hydrochloride; 3-(3-morpholinopropyl)-6-(3-ethyl-4-methylanilino)isocytosine hydrochloride; 3-{4-[4-(6-methyl-4-carboxy-2-pyrimidinyl)-1-piperazinyl]butyl}-6-(3-ethyl-4-methylanilino)uracil; 3-{4-[4-(6-methyl-4-methoxycarbonyl-2-pyrimidinyl)-1-piperazinyl]butyl}-6-(3-ethyl-4-methylanilino)uracil; 3-{4-[4-(4-trifluoromethyl-5-methoxycarbonyl-2-pyrimidinyl)-1-piperazinyl]butyl}-6-(3-ethyl-4-methylanilino)uracil; 3(S)-{5-[(3-pyrrolidinyl)methylamino]pentyl}-6-(3-ethyl-4-methylanilino)uracil; 3-{4-[4-(2-nitro-4-trifluoromethylphenyl)-1-piperazinyl]butyl}-6-(3-ethyl-4-methylanilino)uracil; 3-{4-[4-(4-trifluoromethyl-5-carboxy-2-pyrimidinyl)-1-piperazinyl]butyl}-6-(3-ethyl-4-methylanilino)uracil; 3-{5-[(4-amino-5-cyano-2-pyrimidinyl)amino]pentyl}-6-(3-ethyl-4-methylanilino)uracil; 3-{5-[9-(1,3-dimethyl-2,6-dioxopurinyl)]pentyl}-6-(3-ethyl-4-methylanilino)uracil; 3-[5-(1-carboxy-6-methoxy-2,3,4,9-tetrahydro-1H-b-carboline-2-yl)pentyl]-6-(3-ethyl-4-methylanilino)uracil; 3-[5-(6-methoxy-2,3,4,9-tetrahydro-1H-b-carboline -2-yl)pentyl]-6-(3-ethyl-4-methylanilino)uracil; 3-[5-(2-thiophenecarboxamido) pentyl]-6-(3-ethyl-4-methylanilino)uracil; 3-{5-[1-(4-hydroxy-4-butylpiperidinyl)]pentyl}-6-(3-ethyl-4-methylanilino)uracil; 3-{5-[1-(4-(thiophen-2-yl)-1,2,3,6-tetrahydropyridinyl)] pentyl}-6-(3-ethyl-4-methylanilino)uracil; 3-{5-[1-(4-(benzo[b]thiophen-7-yl)-1,2,3,6-tetrahydropyridinyl)]pentyl}-6-(3-ethyl-4-methylanilino)uracil; 3-{4-[4-(3-carboxy-4-oxo-6-fluoro-7-chloroquinoline-1-yl)butylamino]butyl}-6-(3-ethyl-4-methylanilino)uracil; 3-{5-[1-(4-(4-fluorophenyl)-1,2,3,6-tetrahydropyridinyl)]pentyl}-6-(3-ethyl-4-methylanilino)uracil; 3-{5-[1-(4-(4-chlorophenyl)-1,2,3,6-tetrahydropyridinyl)]pentyl}-6-(3-ethyl-4-methylanilino) uracil; 3-[5-(2-benzo[b]thiophenecarboxamido)pentyl]-6-(3-ethyl-4-methylanilino)uracil; 3-{5-(3-ethoxycarbonyl-4-oxopiperidinyl)pentyl}-6-(3-ethyl-4-methylanilino)uracil; and 3(S)-{5-[(N-tert-butoxycarbonylpyrrolidin-3-yl)methylamino]pentyl}-6-(3-ethyl-4-methylanilino) uracil.

Water Solubility

The compounds described herein have increased water solubility; some of the compounds can form salts, such as with inorganic or organic acids or with alkali metal bases, thus greatly increasing their solubilities. For instance, Compound 35 (3-[4-(N-morpholino)butyl]-6-(3-ethyl-4-methylanilino)uracil, see Example 3 below) has solubility of <1 mg/ml in water at 25° C., but its hydrochloride, Compound 59 (3-[4-(N-morpholino)butyl]-6-(3-ethyl-4-methylanilino)uracil hydrochloride, see Example 3 below), has water solubility of >60 mg/ml in water. The improved water solubilities are a distinct advantage in formulation and in dosing of animals for testing, and for ultimate therapeutic use in humans.

Compound Efficacy

The ability of a test compound to inhibit the activity of DNA Pol IIIC can be tested by using, e.g., a DNA polymerase assay, e.g., as described in Barnes and Brown, Nuc. Acids Res., 6:1203-19 (1979); Trantolo et al., J. Med. Chem., 29:676-681 (1986); Mills et al., J. Bacteriol., 132:641-49 (1977); and Low et al., J. Biol. Chem., 251:1311-25 (1976), all hereby incorporated by reference. This rapid screening method can use natural or recombinant DNA poT IIIC enzyme in a standard DNA polymerase activity assay. By including a test compound in a side-by-side assay with a control, the effect of the test compound on polymerase activity can be assessed. Test compounds with an appropriate level of inhibition of the natural or recombinant bacterial DNA polymerase III are good candidate therapeutics for further evaluation.

Toxicity

The toxicity of the new compounds toward mammalian cells can be evaluated according to standard methods known to those skilled in the art (see, e.g., Gootz, T. D. Clin. Microbiol. Rev., 3:13-31 (1990)). The toxic concentration (or “IC₅₀”) can be determined by using protocols well known in the field of pharmacology. A suitable range of IC₅₀ values for a compound to be considered for further therapeutic evaluation will be greater than the MIC in bacterial cultures, i.e., the therapeutic index should be greater than 10.

Methods of Preparing Compounds

General methods for the preparation of certain N3-substituted-6-anilinouracils have been published (P. Tarantino, C. Zhi, J. Gambino, G. E. Wright and N. C. Brown, 6-Anilinouracil-based Inhibitors of Bacillus subtilis DNA Polymerase III: Antipolymerase and Antimicrobial Structure-Activity Relationships Based on Substitution at Uracil N3, J. Med. Chem., 42:2035-2040 (1999).)

A representative synthesis is shown below:

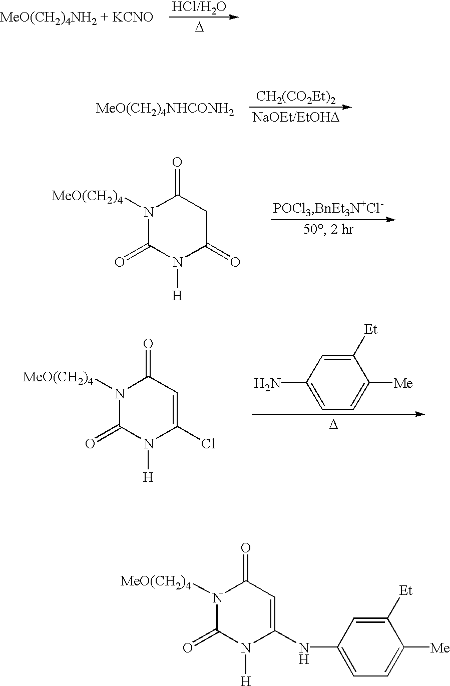

An alternative method is a two-step procedure, illustrated in the following scheme:

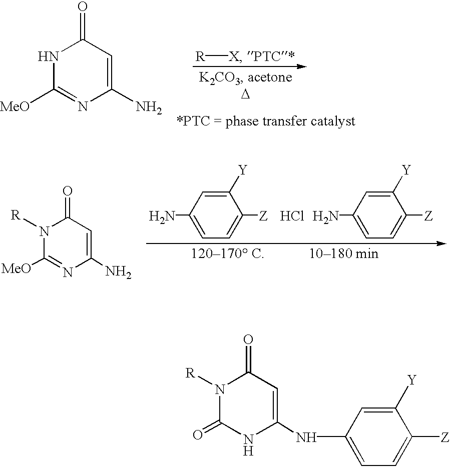

where R—X is a substituted alkyl halide (chloride, bromide or iodide), PTC is a phase transfer catalyst such as benzyltriethylammonium chloride (TBAC) or tetrabutylammonium bromide (TBAB), and Y and Z are, independently, CH₃, CH₂CH₃, CH═CH₂, Cl, Br or I.

Typically, in the first step, a mixture of 6-amino-2-methoxy-4-pyrimidone (1 eq), potassium carbonate (1.2-2 eq), PTC (0.2-1 eq) and alkylating agent (1-5 eq) in acetone or acetonitrile is heated at reflux for 10 hours to 3 days. After cooling to room temperature, the insoluble salts are filtered from the mixture, and the solvent is removed. The residue is purified by chromatography on silica gel with chloroform/methanol as eluent to give first the byproduct 6-amino-4-alkoxy-2-methoxypyrimidine and then the desired 6-amino-2-methoxy-3-alkyl-4-pyrimidone, in approximately equal yields. This first step has been accomplished with simple alkyl halides (see e.g., Müller et al., Synthesis, 1428-1436 (1998)).

In the second step, a mixture of 6-amino-2-methoxy-3-substituted-4-pyrimidone (1.0 eq), substituted-aniline hydrochloride (1.2-2.5 eq), and a few drops of the substituted aniline (ca. 0.1-1 eq) is heated at 120-170° C. for between 10 minutes to 3 hours. After cooling to room temperature, water is added, and the mixture is extracted with chloroform. The combined organic layers are dried over anhydrous magnesium sulfate. The solvent is removed under reduced pressure, and the residue is purified by chromatography on silica gel with chloroform:methanol as eluent to give the target compounds, 3-alkyl-6-anilinouracils, in good yields.

An alternate method for carrying out the first step of the above-described two-step procedure is illustrated below:

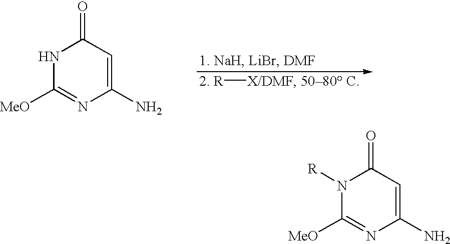

In this alternate method, sodium hydride (1.2 eq) is added to a mixture of 6-amino-2-methoxy-4-pyrimidone (1 eq) in N,N-dimethylformamide (DMF) at 0° C. Lithium bromide (1.2-2.0 eq) is added, and the mixture is stirred for 1 hour at room temperature. The mixture is added dropwise to a solution of the alkylating agent (1.5 eq) in DMF at 50-80° C., and the reaction mixture is stirred at 50-80° C. for 3-10 hours. After cooling to room temperature, the solvent is removed. The residue is purified by chromatography on silica gel with chloroform:methanol as eluent, to give 6-amino-2-methoxy-3-substituted-4-pyrimidones.

An alternative method for synthesizing N3-substituted pyrimidines is shown below:

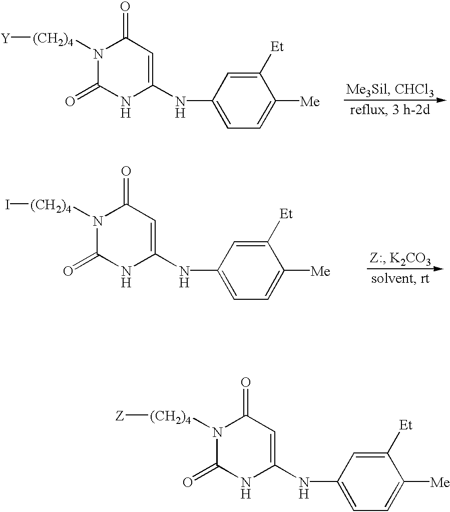

where Y is OCH₃, OH, or OCOCH₃, and Z: is a nucleophile.

In the first step of this method, trimethylsilyl iodide (2-5 eq) is added to a stirred solution of 3-(4-methoxybutyl)-6-(3-ethyl-4-methylanilino)uracil [or 3-(4-hydroxybutyl)-6-(3-ethyl-4-methylanilino)uracil, or 3-(4-acetoxybutyl)-6-(3-ethyl-4-methylanilino)uracil] (1 eq) in dry chloroform. The reaction mixture is stirred at reflux for 3 hours-2 days, until disappearance of starting material. Methanol and sodium sulfite are then added to the brown-purple solution. After stirring at room temperature for 10 minutes, the mixture is filtered, and the solvent is removed in vacuo. The residue is purified by chromatography on silica gel with chloroform:methanol (2:98-7:93) as eluent to give 3-(4-iodobutyl)-6-(3-ethyl-4-methylanilino)uracil in high yield (80-95%).

In the second step, a mixture of 3-(4-iodobutyl)-6-(3-ethyl-4-methylanilino)uracil, potassium carbonate, and nucleophile Z: in a solvent (acetone, acetonitrile or DMF) is stirred at room temperature. Once the reaction is complete, e.g., as monitored by thin layer chromatography, the solution is concentrated in vacuo, and water is added. The mixture is extracted with chloroform, and the extracts are dried over sodium sulfate. After removal of chloroform, the residue is purified by chromatography on silica gel using chloroform:methanol as eluent to give the products.

Methods for preparing the compounds disclosed herein are further described in Zhi et al., U.S. S. No. 60/298,436, filed on Jun. 15, 2001.

Detection of Infections

Gram-positive bacterial infections can be detected by any standard method (e.g., Gram staining). Mycoplasmal infections can also be detected using standard techniques. Once identified, infected cell cultures can be treated with compounds of the invention to inhibit the growth of the bacteria. In addition, infected subjects (e.g., animals and humans) can be treated by administering compounds of the invention. Subjects (e.g., animals and humans) at risk for bacterial infection can also be treated prophylactically using compounds of the invention; in these cases, bacterial infections can be inhibited or reduced.

Therapeutic Administration of Compounds

The compounds described herein are useful for the treatment of infections in humans caused by Gram-positive bacteria, including strains resistant to common antibiotic drugs. The compounds are also useful for the treatment of mycoplasmal infections in humans caused by various species of the genera Mycoplasma and Ureaplasma. They are also useful for the treatment of related Gram-positive bacterial infections and mycoplasmal infections in animals such as pigs, cows, horses, goats, chickens, turkeys, sheep, dogs, cats, rats, mice, and rabbits, and for eliminating or avoiding bacterial or mycoplasmal infections of eukaryotic cell cultures.

The compounds of the invention can be formulated for pharmaceutical, veterinary, and tissue culture use, optionally together with an acceptable diluent, carrier, or excipient and/or in unit dosage form. In using the compounds of the invention, conventional pharmaceutical, veterinary, or culture practice can be employed to provide suitable formulations or compositions, all of which are encompassed by the pharmaceutical compositions of this invention.

For human or animal use, the formulations of this invention can be administered by parenteral administration, for example, intravenous, subcutaneous, intramuscular, intraorbital, ophthalmic, intraventricular, intracranial, intracapsular, intraspinal, intracistemal, or intraperitoneal administration, or by intranasal, aerosol, scarification, oral, buccal, rectal, vaginal, or topical administration. The formulations of this invention can also be administered by the use of surgical implants which release the compounds of the invention, either as a bolus or slowly over a pre-selected period of time.

Without limitation, parenteral formulations can be, for example, in the form of liquid solutions or suspensions; for oral administration, formulations can be, for example, in the form of tablets, capsules, liquid solutions and suspensions (wherein such solutions and suspensions are particularly for formulations intended for pediatric use); and for intranasal administration, the formulations can be, for example, in the form of powders, nasal drops, or aerosols. Other suitable formulations for parenteral, oral, or intranasal delivery of the compounds of this invention will be well known to those of ordinary skill in the art.

Methods well known in the art for making formulations can be found in, for example, “Remington's Pharmaceutical Sciences.” Formulations for parenteral administration may contain as excipients sterile water or saline, polyalkylene glycols such as polyethylene glycol, oils of vegetable origin, hydrogenated naphthalenes, or biocompatible, biodegradable lactide polymers. Polyoxyethylene-polyoxypropylene copolymers can be used to control the release of the present factors. Other potentially useful parenteral delivery systems for the compounds of the invention include ethylene-vinyl acetate copolymer particles, osmotic pumps, implantable infusion systems, and liposomes. Formulations for inhalation may contain lactose as an excipient, or can be aqueous solutions containing, for example, polyoxyethylene-9-lauryl ether, glycocholate and deoxycholate, or can be oily solutions for administration in the form of nasal drops, or can be gels to be applied intranasally. Formulations for parenteral administration may also include glycocholate for buccal administration, methoxysalicylate for rectal administration, or citric acid for vaginal administration.

The concentration of the compound in the formulations of the invention will vary depending upon a number of factors, including the dosage to be administered, and the route of administration. In general, the compounds of the invention can be provided in an aqueous physiological buffer solution containing about 0.1 to 10% w/v compound for parenteral administration. General dose ranges are from about 0.01 mg/kg to about 1 g/kg of body weight per day, e.g., from about 0.01 mg/kg to 100 mg/kg or 0.1 ug/kg to 50 mg/kg of body weight per day. The dosage to be administered depends upon the type and extent of progression of the infection being addressed, the overall health of the patient, and the route of administration. For topical and oral administration, formulations and dosages can be similar to those used for other antibiotic drugs, e.g., erythromycin.

In one embodiment, a compound or composition of the invention is administered to an animal (e.g., swine, cow, horse, chicken, or other commercially relevant livestock) or to a human patient who has been diagnosed with a mycoplasmal or Gram-positive bacterial infection. The compounds can also be administered to the animal (e.g., a human) to inhibit or reduce the likelihood of a mycoplasmal or Gram-positive bacterial infection, particularly in an animal susceptible to such infections (including, without limitation, a human patient who is immunodeficient or immunocompromised, or one who has recently undergone a medical procedure). In other embodiments, cultured eukaryotic cells, either those that have mycoplasmal or Gram positive bacterial infections, are treated with the new compositions, or the compositions are added to inhibit or reduce the likelihood of such infections (e.g., prophylactic treatment).

The compounds can be administered both prophylactically and after infection has occurred. Prophylaxis can be most appropriate for immunocompromised animal and human patients and for animals and patients following surgery or dental procedures. This list of relevant conditions for application of the methods of the invention is not intended to be limiting, and any appropriate infection responsive to the compounds can be treated using the methods and/or compounds described herein.

### EXAMPLES

The following specific examples are to be construed as merely illustrative, and not limitative of the remainder of the disclosure in any way.

### Example 1

### Enzyme Assays and Determination of Inhibitor K₁ Values

DNA pol activity was assayed as described in Barnes et al., Nuc. Acids Res., 6:1203-19 (1979), using activated “nicked” calf-thymus DNA as template primer, 10 pM [³H-methyl]-dTTP as the labelled dNTP substrate, and dATP, dCTP, and dGTP at 25 μM each.

Inhibitory activity of the compounds is determined by measuring the ability of the agent to inhibit enzyme-catalyzed incorporation of [³H]-dTMP into nicked calf thymus DNA in the absence of the competitor dGTP or dATP (“truncated assay”) (see G. E. Wright and N. C. Brown, Inhibition of Bacillus subtilis DNA Polymerase III by Arylhydrazinopyrimidines: Novel Properties of 2-Thiouracil Derivatives, Biochim. Biophys. Acta 432:37-48 (1976)). Purified pol IIIC is added to a buffered solution containing Mg²⁺, DTT, glycerol, nicked calf thymus DNA, saturating concentrations of dATP, dCTP, dTTP and [³H]-dTTP. Reaction mixtures are incubated at 30° C. for 10 minutes, quenched and filtered, and radioactivity in the acid-insoluble material measured by scintillation counting. Experiments are done in triplicate. Inhibitors are assayed by addition of several dilutions of a stock solution of inhibitor (DMSO or water, depending on solubility) before enzyme addition. Typically compounds are tested at five concentrations to estimate the K, value. The truncated assay, i.e. exclusion of the competitive substrate dGTP or dATP, depending upon whether the compound is a uracil derivative or an isocytosine derivative, respectively, allows for the direct determination of apparent inhibitor constants (K,) in this assay system.

### Example 2

### Inhibition of Bacterial Growth with N3-substituted 6-anilinopyrimidines

Each compound is assayed against a panel of Bacillus, Enterococcus and Staphylococcus, and a Gram-negative bacterium Escherichia coli as negative control, grown in appropriate plate media solidified with 1.3% agar-agar. Stock solutions of the compounds in dimethylsulfoxide or water, depending on solubility, are added to sterile medium at a temperature of 60° C. This stock mixture is diluted with drug-free medium and used to make a series of Petri plates containing inhibitor in a series of two-fold serial dilutions, from about 80 to 0.625 μg/mL. One tenth mL of diluted bacteria containing 500-1000 colony-forming units (CFU) are plated and spread, and the plates incubated at 37° C. for 24 hours. MIC (minimum inhibitory concentration) is equivalent to the lowest concentration at which growth, i.e. colony formation, is not observed.

As the data in Table 1 show, the N3-substituents can increase the potency of the 6-anilinouracils and 6-anilinoisocytosines in inhibiting the model enzyme Pol IIIC from B. subtilis. Some of the compounds shown in Table 1 inhibit the growth of Gram-positive bacteria, but do not inhibit the growth of Gram-negative bacteria.

TABLE 1
Pol IIIC inhibition and antibacterial activity of N3-substituted-6-
anilinopyrimidines

| | | K₁ (microM) | | MIC (microg/ml) | | |
|---|---|---|---|---|---|---|
| | | B.subtilis | B. | S.aureus | E. | E. |
| Compound | R | pol IIIC | subtilis | (Smith) | fecalis | fecium |
| 5 | CH₂CH₂OCH₂CH₂OBn | 0.056 | <1.25 | 5 | 5 | 10 |
| 6 | (CH₂)₄CO₂Et | 0.076 | <1.25 | 2.5 | 5 | <1.25 |
| 7 | CH₂CH═CHCO₂CH₃ | 0.32 | 2.5 | 10 | 20 | 5 |
| 8 | CH₂CH₂CH₂COCH₃ | 0.061 | <1.25 | 5 | 5 | 5 |
| 10 | (CH₂)₃CO₂Et | 0.1 | <1.25 | 2.5 | 5 | 2.5 |
| 11 | CH₂CONEt₂ | 0.45 | 2.5 | 10 | 20 | 20 |
| 12 | CH₂CH₂OCH₂CH₂OMe | 0.045 | 2.5 | 2.5 | 5 | 5 |
| 13 | CH₂CCCH₂OH | 0.068 | 5 | 5 | 10 | 10 |
| 14 | CH₂CH₂NEt₂ | 0.53 | 10 | 20 | 20 | 20 |
| 15 | CH₂COCH₃ | 0.303 | 2.5 | 10 | 20 | 10 |
| 16 | CH₂CH₂NHSO₂CH₃ | 0.29 | 5 | 10 | 20 | 10 |
| 17 | (CH₂)2—N[(CH₂)₄]O | 0.09 | <1.25 | 5 | 5 | 2.5 |
| 18 | (CH₂)₈OH | 0.026 | 1.25 | 2.5 | 2.5 | 2.5 |
| 19 | (CH₂)₃CN | 0.14 | 2.5 | 2.5 | 5 | 5 |
| 20 | (CH₂)₄CN | 0.094 | 2.5 | 2.5 | 2.5 | 5 |
| 21 | (CH₂)₂O(CH₂)₂O(CH₂)₂OH | 0.087 | 2.5 | 10 | 10 | 10 |
| 22 | (CH₂)₄OAc | 0.058 | 1.25 | 2.5 | 5 | 5 |
| 23 | CH₂CH₂CH₂CH(OH)CH₂OH | 0.096 | 10 | 20 | 20 | 20 |
| 24 | (CH₂)₃—N[(CH₂)₄]O | 0.11 | 2.5 | 10 | 10 | 5 |
| 25 | CH₂CH(OH)CH₃(S) | 0.11 | <1.25 | 5 | 5 | 2.5 |
| 27 | (CH₂)₆OH | 0.049 | <1.25 | 2.5 | 2.5 | 5 |
| 29 | CH₂CH₂OCH₂CH₂OH | 0.125 | <1.25 | 5 | 5 | 5 |
| 31 | (CH₂)₃CO₂H | 0.057 | >20 | 10 | >20 | 20 |
| 32 | CH₂CH═CHCH₂—OH | 0.051 | 1.25 | 5 | 5 | 2.5 |
| 35 | (CH₂)₄—N[(CH₂)₄]O | 0.048 | 2.5 | 5 | 5 | 2.5 |
| 36 | (CH₂)₄N[(CH₂)₄]S | 0.068 | 5 | 10 | 5 | 5 |
| 37 | (CH₂)₄N[(CH₂)₅]-4-OH-4- | 0.032 | 1.25 | 2.5 | 2.5 | 2.5 |
| | ClCF₃Ph | | | | | |
| 38 | (CH₂)₄N[(CH₂)₅]-4-OH-4-(pClPh) | 0.025 | 5 | 5 | 5 | 5 |
| 39 | (CH₂)₂—N[(CH₂)₄]N—COPh | 0.077 | 2.5 | 10 | 10 | 5 |
| 40 | (CH₂)₄N[(CH₂)₄]N—CO-2-furyl | 0.043 | 5 | 5 | 5 | 10 |
| 41 | (CH₂)₂—N[(CH₂)₄]N—CH₂Ph | 0.034 | 5 | >20 | 10 | 5 |
| 44 | (CH₂)₄N[c-2,6-diMe-morpholine] | 0.036 | 2.5 | >20 | >20 | 20 |
| 45 | (CH₂)₄N[(CH₂)₄]N-(2- | 0.043 | 2.5 | 10 | 5 | 5 |
| | pyrimidinyl) | | | | | |
| 46 | (CH₂)₄N[(CH₂)₄]N-(pF-Ph) | 0.07 | 5 | 20 | 10 | 5 |
| 47 | (CH₂)₄OCON[(CH₂)₄]O | 0.071 | <1.25 | 5 | 5 | 5 |
| 48 | (CH₂)₄NH—SO₂-(2-thienyl) | 0.075 | 2.5 | 5 | 5 | 2.5 |
| 49 | (CH₂)₄NH—CO-cC₃H₅ | 0.082 | 5 | 5 | 5 | 2.5 |
| 50 | (CH₂)₄NHCOCH₂CI | 0.035 | 2.5 | 5 | 5 | 10 |
| 51 | (CH₂)₄—OCOCH₂Cl | 0.04 | 0.313 | 10 | 2.5 | 5 |
| HB-EMAU | CH₂CH₂CH₂CH₂OH | 0.07 | <1.25 | 5 | 5 | 5 |
| 60 | 4-Me-6-CO₂H-2-pyrimidinyl.HCl | 0.016 | 40 | 40 | 40 | 40 |
| 61 | 4-CH₃-6-CO₂Me-2-pyrimidinyl | 0.066 | 10 | 40 | 20 | 20 |
| 62 | 4-CF₃-5-CO₂Me-2-pyrimidinyl | 0.55 | 80 | 80 | 80 | 80 |
| 63 | Pyrrolidinyl-3-methyl | 0.112 | 20 | 40 | 80 | 80 |
| 64 | N(2-NO₂-4-CF₃Ph)piperazinyl | 58.8 | 80 | 80 | 80 | 80 |
| 65 | 3-CF₃-4-CO₂H-2-pyrimidinyl | 0.113 | 80 | 80 | 80 | 40 |
| 66 | 3-NH₂-4-CN-2-pyrimidinyl | 39.5 | 80 | 80 | 80 | 80 |
| 67 | 9-caffeinyl | 0.06 | 2.5 | 10 | 20 | 10 |
| 68 | 2-CO₂H-7-OMe-b-carboline | 0.288 | 20 | 40 | 80 | 80 |
| 69 | 7-OMe-b-carboline | 0.109 | 5 | 10 | 20 | 20 |
| 70 | See structure | 0.065 | 1.25 | 10 | 5 | 5 |
| 71 | See structure | 2.1 | 5 | 10 | 10 | 10 |
| 72 | See structure | 0.077 | 5 | 20 | 20 | 40 |
| 73 | See structure | 0.08 | 10 | 10 | >80 | >80 |
| 74 | 1-(ClFquin)-AB-EMAU | 0.063 | 20 | 20 | 20 | 20 |
| 75 | See structure | 0.098 | 5 | 5 | 5 | 5 |
| 76 | See structure | 0.087 | 5 | 5 | 80 | 80 |
| 77 | See structure | 0.48 | 80 | 80 | 80 | 80 |
| 78 | See structure | 0.25 | 80 | 80 | 80 | 80 |
| 79 | Boc-pyrrolidinyl3-methyl | 0.12 | 20 | 20 | 40 | 40 |
| 80 | See structure | 0.024 | 2.5 | 10 | 5 | 5 |
| 81 | See structure | 0.034 | 1.25 | 20 | 10 | 10 |
| 82 | See structure | 0.029 | 10 | 20 | 20 | 20 |

### Example 3

### Organic Synthesis of Compounds of the Invention

General methods for the preparation of 6-amino-2-methoxy-3-substituted-4-pyrimidones

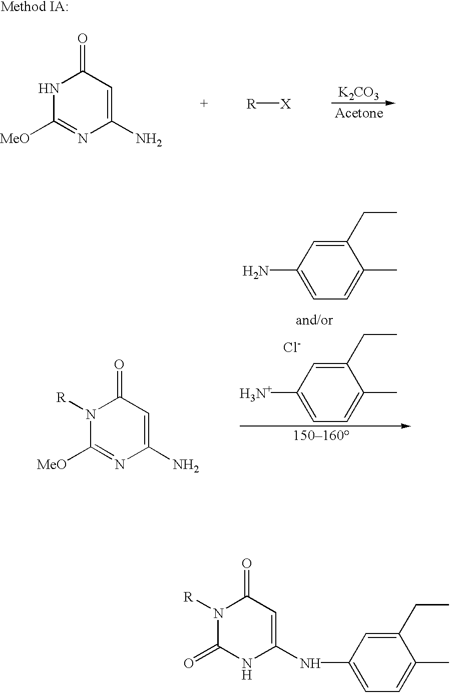

A mixture of 6-amino-2-methoxy-4-pyrimidone (1 eq), potassium carbonate (1.2-2 eq), benzyltriethylammonium chloride (0.2-1 eq) and alkylating agent (1-5 eq) in acetone (or acetonitrile) was heated at 50-100° C. for 10 hours-3 days. After cooling to room temperature, the insoluble salts were filtered off and the solvent was removed. The residue was purified by chromatography on silica gel with chloroform/methanol as eluent to give 6-amino-2-methoxy-3-substituted-4-pyrimidones.

Method IB:

Sodium hydride (1.2 eq) was added to a mixture of 6-amino-2-methoxy-4-pyrimidone (1 eq) in DMF at 0° C. Then lithium bromide (1.2-2.0 eq) was added to the mixture and stirred for 1 hour at room temperature. The mixture was added dropwise to the solution of alkylating agent (1.5 eq) in DMF at 50-80° C. and the reaction mixture was stirred at 50-80° C. for 3-10 hours. After cooling to room temperature, the solvent was removed. The residue was purified by chromatography on silica gel with CHCl₃/MeOH as eluent, to give 6-amino -2-methoxy-3-substituted-4-pyrimidones.

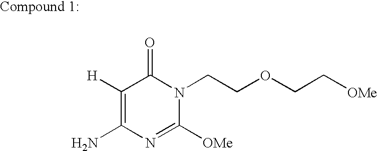

6-Amino-2-methoxy-3-[2-(2-methoxyethoxy)ethyl]-4-pyrimidone

The mixture of 6-amino-2-methoxy-4-pyrimidone (1 eq), potassium carbonate (1.5 eq), benzyltriethylammonium chloride (0.3 eq) and 2-(2-methoxyethoxy)ethyl bromide (1.5 eq) in acetone was heated at reflux overnight. After cooling to room temperature, the insoluble salts were filtered off and the solvent was removed. The residue was purified by chromatography on silica gel with chloroform:methanol (98:2-95:5) as eluent, to give 6-amino-2-methoxy-3-[2-(2-methoxyethoxy)ethyl]-4-pyrimidone (yield 42%) as a white solid. 300 MHz ¹H NMR (DMSO-d₆): δ 3.22 (s, 3H, CH₃O), 3.38 (m, 2H, CH₂O), 3.47 (m, 4H, 2×CH₂), 3.85 (s, 3H, CH₃O), 3.91 (t, 2H, CH₂N), 4.81 (s, 1H, C₅—H), 6.36 (s, 2H, NH₂) ppm.

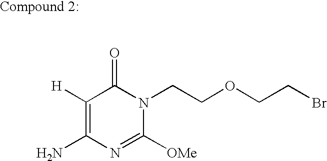

6-Amino-2-methoxy-3-[2-(2-bromoethoxy)ethyl]-4-pyrimidone

Method IA yielded 25% of Compound 2. 300 MHz ¹H NMR (DMSO-d₆): δ 3.53 (m, 4H, BrCH₂ and CH₂O), 3.70 (t, 2H, CH₂O), 3.88 (s, 3H, CH₃O), 3.93 (t, 2H, CH₂N), 4.84 (s, 1H, C₅—H), 6.42 (s, 2H, NH₂) ppm.

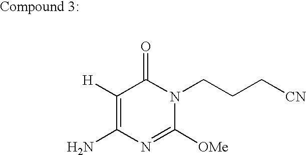

6-Amino-2-methoxy-3-(3-cyanopropyl)-4-pyrimidone

Sodium hydride (1.2 eq) was added to a mixture of 6-amino-2-methoxypyrimidin-4-one (1 eq) in DMF at 0° C. Then lithium bromide (1.4 eq) was added, and the mixture was stirred for 1 hour at room temperature. The mixture was added dropwise to a solution of 4-bromo-1-butyronitrile (1.5 eq) in DMF at 80° C., and the reaction mixture was stirred at 80° C. for 5 hours. After cooling to room temperature, the solvent was removed in vacuo. Water was added and the mixture was extracted with chloroform, and the organic extracts were dried over sodium sulfate. After removal of chloroform, the residue was purified by chromatography on silica gel using chloroform:methanol as eluent to give 6-amino-2-methoxy-3-(3-cyanopropyl)pyrimidin-4(3H)-one (yield 65%) as a white solid. 300 MHz ¹H NMR (DMSO-d₆): δ 1.78 (m, 2H, CH₂), 2.50 (t, 2H, CH₂CN) 3.84 (t, 2H, CH₂N), 3.88 (s, 3H, CH₃O), 4.83 (s, 1H, C₅—H), 6.44 (s, 2H, NH₂) ppm.

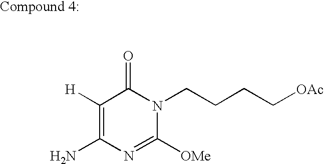

6-Amino-2-methoxy-3-(4-acetoxybutyl)pyrimidin-4(3H)-one

Sodium hydride (1.2 eq) was added to the mixture of 6-amino-2-methoxy-4-pyrimidone (1 eq) in DMF at 0° C. Then lithium bromide (1.5 eq) was added to the mixture and stirred for 1 hour at room temperature. The mixture was added dropwise to a solution of 4-bromo-1-acetoxybutane (1.5 eq) in DMF at 50° C. Workup and chromatography gave 6-amino-2-methoxy-3-(4-acetoxybutyl)-4-pyrimidone (yield 54%) as a white solid. 300 MHz ¹H NMR (DMSO-d₆): δ 1.52 (m, 4H, 2×CH₂), 2.0 (s, 3H, CH₃CO) 3.76 (t, 2H, CH₂O), 3.88 (s, 3H, CH₃N),), 4.0 (t, 2H, CH₂O), 4.82 (s, 1H, C₅—H), 6.41 (s, 2H, NH₂)ppm.

General Method for the Preparation of 3-substituted-6-anilinouracils

Method II:

A stirred mixture of 6-amino-2-methoxy-3-substituted-4-pyrimidone (1.0 eq), 3-ethyl-4-methylaniline hydrochloride (1.1-1.5 eq), and a few drops of 3-ethyl-4-methylaniline was heated at 120-170° C. for 10 minute-3 hours. After cooling to room temperature, water was added and the mixture extracted with chloroform. The combined organic layers were dried over anhydrous magnesium sulfate. The solvent was removed under reduced pressure, and the residue was purified by chromatography on silica gel with chloroform:methanol as eluent, to give target compounds.

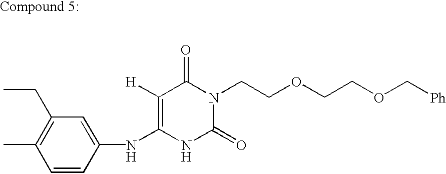

3-[2-(2-Benzyloxyethoxy)ethyl]-6-(3-ethyl-4-methylanilino)uracil

A mixture of 6-amino-2-methoxy-3-[2-(2-benzyloxyethoxy)ethyl]-4-pyrimidone (430 mg, 1.35 mmol) and 3-ethyl-4-methylaniline hydrochloride (254 mg, 1.48 mmol), and a few drops of 3-ethyl-4-methylaniline was heated at 160° C. for 3 hours. After cooling to room temperature, water (15 ml) was added and the mixture extracted with chloroform (3×40 ml). The combined organic layers were dried over anhydrous magnesium sulfate. The solvent was removed under reduced pressure, and the residue was purified by chromatography on silica gel with chloroform:methanol (99:1-97:3) as eluent, to give 410 mg (72% yield) of 3-[2-(2-benzyloxyethoxy)ethyl]-6-(3-ethyl-4-methylanilino)uracil. Crystallization from ethanol gave white crystals. 300 MHz ¹H NMR (DMSO-d₆): δ 1.13 (t, 3H, CH₃CH₂), 2.24 (s, 3H, CH₃Ar), 2.57 (q, 2H, CH₂Ar), 3.53 (m, 6H, 3×CH₂), 3.88 (t, 2H, CH₂N), 4.47 (s, 2H, PhCH₂), 4.72 (s, 1H, C₅—H), 6.92-7.15 (m, 3H, Ar—H), 7.25-7.36 (m, 5H, Ph—H), 8.16 (s, 1H, NH), 10.49 (s, 1H, NH) ppm.

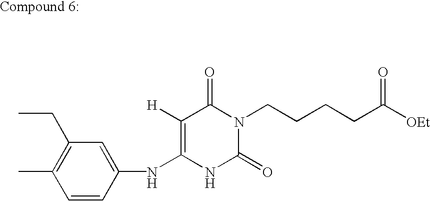

3-(4-Ethoxycarbonylbutyl)-6-(3-ethyl-4-methylanilino)uracil

A mixture of 6-amino-2-methoxy-3-[4-(ethoxycarbonyl)butyl]-4-pyrimidone (608 mg, 2.26 mmol) and 3-ethyl-4-methylaniline hydrochloride (430 mg, 2.50 mmol), and a few drops of 3-ethyl-4-methylaniline was heated at 160° C. for 3 hours. Workup gave 632 mg (75% yield) of 3-[4-(ethoxycarbonyl)butyl]-6-(3-ethyl-4-methylanilino)uracil. Crystallization from ethanol gave white crystals. 300 MHz ¹H NMR (DMSO-d₆): δ 1.11-1.19 (m, 6H, 2×CH₃), 1.49 (m, 4H, 2×CH₂), 2.24 (s, 3H, CH₃Ar), 2.30 (t, 2H, CH₂CO₂Et), 2.57 (q, 2H, CH₂Ar), 3.69 (t, 2H, CH₂N), 4.04 (q, 2H, CO₂CH₂), 4.72 (s, 1H, C₅—H), 6.92-7.15 (m, 3H, Ar—H), 8.16 (s, 1H, NH), 10.48 (s, 1H, NH) ppm.

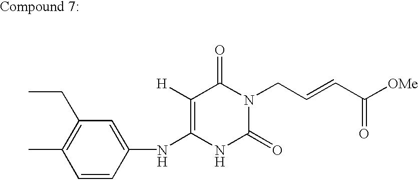

3-[3-(Methoxycarbonyl)-2-propenyl]-6-(3-ethyl-4-methylanilino)uracil

A mixture of 6-amino-2-methoxy-3-[3-(methoxycarbonyl)-2-propenyl)-4-pyrimidone (500 mg, 2.09 mmol) and 3-ethyl-4-methylaniline hydrochloride (450 mg, 2.62 mmol), and a few drops of 3-ethyl-4-methylaniline was heated at 165° C. for 3 hours. Workup gave 158 mg (22% yield) of 3-[3-(methoxycarbonyl)-2-propenyl)-6-(3-ethyl-4-methylanilino)uracil. Crystallization from ethanol gave light yellow solid. 300 MHz ¹H NMR (DMSO-d₆): δ 1.14 (t, 3H, CH₃CH₂Ar), 2.24 (s, 3H, CH₃Ar), 2.58 (q, 2H, CH₂Ar), 3.65 (s, 3H, CO₂CH₃), 4.47 (d, 2H, CH₂N), 4.75 (s, 1H, C₅—H), 5.73 (d, 1H, C═CHCO₂Me), 6.85 (dt, 1H, CH₂CH═C), 6.94-7.17 (m, 3H, Ar—H), 8.21 (s, 1H, NH), 10.60 (s, 1H, NH) ppm.

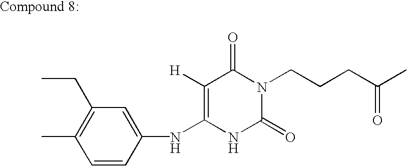

3-(4-Oxopentyl)-6-(3-ethyl-4-methylanilino)uracil

A mixture of 6-amino-2-methoxy-3-(4-oxopentyl)-4-pyrimidone (306 mg, 1.36 mmol) and 3-ethyl-4-methylaniline hydrochloride (260 mg, 1.51 mmol), and a few drops of 3-ethyl-4-methylaniline was heated at 160° C. for 3 hours. Workup gave 92 mg (21% yield) of 3-(4-oxopentyl)-6-(3-ethyl-4-methylanilino)uracil. Crystallization from ethanol gave white crystals. 300 MHz ¹H NMR (CDCl₃): δ 1.17 (t, 3H, CH₃CH₂), 1.90 (m, 2H, CH₂), 2.09 (s, 3H, CH₃CO), 2.28 (s, 3H, CH₃Ar), 2.45 (t, 2H, CH₂CO), 2.58 (q, 2H, CH₂Ar), 3.87 (t, 2H, CH₂N), 5.09 (s, 1H, C₅—H), 6.89-7.11 (m, 3H, Ar—H), 7.46 (s, 1H, NH), 10.10 (s, 1H, NH) ppm.

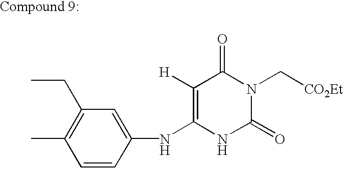

3-(Ethoxycarbonylmethyl)-6-(3-ethyl-4-methylanilino)uracil

A mixture of 6-amino-2-methoxy-3-(ethoxycarbonylmethyl)-4-pyrimidone (24 mg, 0.11 mmol) and 3-ethyl-4-methylaniline hydrochloride (20 mg, 0.12 mmol), and one drop of 3-ethyl-4-methylaniline was heated at 165° C. for 30 minutes. Workup gave 31 mg (89% yield) of 3-(ethoxycarbonylmethyl)-6-(3-ethyl-4-methylanilino)uracil as white crystals. 300 MHz ¹H NMR (DMSO-d₆): δ 1.05-1.20 (m, 6H, 2×CH₃), 2.24 (s, 3H, CH₃Ar), 2.57 (q, 2H, CH₂Ar), 4.04 (q, 2H, CO₂CH₂), 4.38 (s, 2H, CH₂N), 4.72 (s, 1H, C₅—H), 6.92-7.15 (m, 3H, Ar—H), 8.20 (s, 1H, NH), 10.62 (s, 1H, NH) ppm.

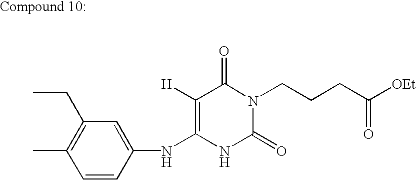

3-[3-(Ethoxycarbonyl)propyl]-6-(3-ethyl-4-methylanilino)uracil

A mixture of 6-amino-2-methoxy-3-[3-(ethoxycarbonyl)propyl]-4-pyrimidone (143 mg, 0.56 mmol) and 3-ethyl-4-methylaniline hydrochloride (102 mg, 0.59 mmol), and a few drops of 3-ethyl-4-methylaniline was heated at 165° C. for 1 hour. Workup gave 186 mg (92% yield) of 3-[3-(ethoxycarbonyl)propyl]-6-(3-ethyl-4-methylanilino)uracil. Crystallization from ethanol gave white crystals. 300 MHz ¹H NMR (DMSO-d₆): δ 1.16 (m, 6H, 2×CH₃), 1.75 (m, 2H, CH₂), 2.24 (s, 3H, CH₃Ar), 2.27 (t, 2H, CH₂CO₂Et), 2.57 (q, 2H, CH₂Ar), 3.72 (t, 2H, CH₂N), 4.03 (q, 2H, CO₂CH₂), 4.72 (s, 1H, C₅—H), 6.92-7.16 (m, 3H, Ar—H), 8.09 (s, 1H, NH), 10.40 (s, 1H, NH) ppm.

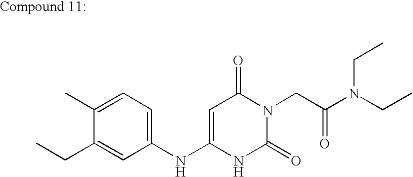

3-[(N,N-Diethylaminocarbonyl)methyl]-6-(3-ethyl-4-methylanilino)uracil

Method II gave the product in 59% yield. 300 MHz ¹H NMR (DMSO-d₆): 10.53 (s, 1H, NH), 8.19 (S, 1H, NH), 6.94-7.15 (m, 3H, Ar—H), 4.72 (s, 1H, C₅—H), 4.49 (s, 2H, NCH₂), 3.26 (m, 4H, N(CH₂)₂), 2.59 (q, 2H, ArCH₂), 2.24 (s, 3H, ArCH₃), 0.98-1.17 (m, 9H, 3×CH₃) ppm.

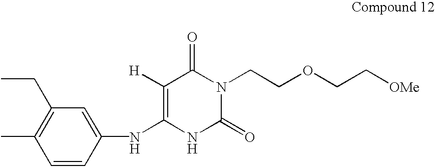

3-[2-(2-Methoxyethoxy)ethyl]-6-(3-ethyl-4-methylanilino)uracil

Method II gave the product in 74% yield. 300 MHz ¹H NMR (DMSO-d₆): δ 1.13 (t, 3H, CH₃CH₂), 2.24 (s, 3H, CH₃Ar), 2.57 (q, 2H, CH₂Ar), 3.22 (s, 3H, CH₃O), 3.37 (m, 2H, CH₂O), 3.49 (m, 4H, 2×CH₂O), 3.85 (t, 3H, CH₂N), 4.70 (s, 1H, C₅—H), 6.92-7.15 (m, 3H, Ar—H), 8.12 (s, 1H, NH), 10.40 (s, 1H, NH) ppm.

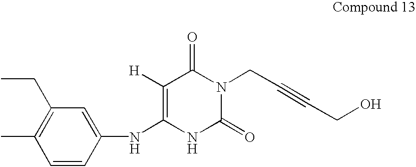

3-(4-Hydoxy-2-butynyl)-6-(3-ethyl-4-methylanilino)uracil

Method II gave the product in 41% yield. 300 MHz ¹H NMR (DMSO-d₆): 10.60 (s, 1H, NH), 8.19 (S, 1H, NH), 6.93-7.13 (m, 3H, Ar—H), 5.12 (t, 1H, OH), 4.72 (s, 1H, C₅—H), 4.47 (m, 2H, NCH₂), 4.02 (d, 2H, OCH₂), 2.57 (q, 2H, ArCH₂), 2.23 (s, 3H, ArCH₃), 1.14 (t, 3H, ArCH₂CH₃)ppm.

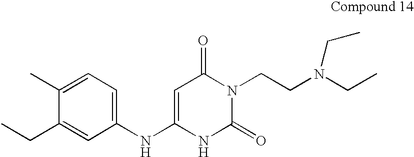

3-[2-(N,N-diethylamino)ethyl]-6-(3-ethyl-4-methylanilino)uracil

Method II gave the product in 61% yield. 300 MHz ¹H NMR (DMSO-d₆): 10.34 (s, 1H, NH), 8.13 (S, 1H, NH), 6.87-7.16 (m, 3H, Ar—H), 4.72 (s, 1H, C₅—H), 3.77 (t, 2H, NCH₂), 3.22 (m, 2H, CH₂N), 2.58 (q, 2H, ArCH₂), 2.43 (m, 4H, 2×NCH₂), 2.20 (s, 3H, ArCH₃), 1.16 (t, 3H, ArCH₂CH₃), 1.00 (m, 6H, 2×CH₃) ppm.

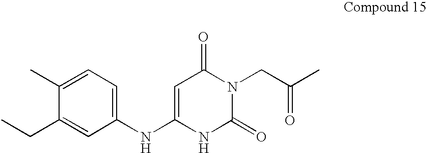

3-(2-Oxopropyl)-6-(3-ethyl-4-methylanilino)uracil

Method II gave the product in 59% yield. 300 MHz ¹H NMR (DMSO-d₆): 10.55 (s, 1H, NH), 8.20 (S, 1H, NH), 6.92-7.13 (m, 3H, Ar—H), 4.73 (s, 1H, C₅—H), 4.50 (s, 2H, NCH₂), 2.59 (q, 2H, ArCH₂), 2.22 (s, 3H, ArCH₃), 2.10 (s, 3H, CH₃CO), 1.14 (t, 3H, ArCH₂CH₃)ppm.

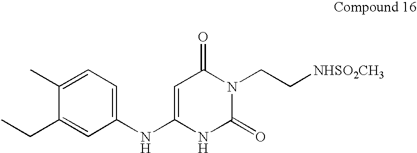

3-[2-(Methanesulfonylamino)ethyl]-6-(3-ethyl-4-methylanilino)uracil

Method II gave the product in 92% yield. 300 MHz ¹H NMR (DMSO-d₆): 10.53 (s, 1H, NH), 8.18 (S, 1H, NH), 7.16 (S, 1H, NH), 6.90-7.13 (m, 3H, Ar—H), 4.73 (s, 1H, C₅—H), 3.83 (t, 2H, NCH₂), 3.09 (m, 2H, CH₂NH), 2.88 (s, 3H, SO₂CH₃), 2.59 (q, 2H, ArCH₂), 2.20 (s, 3H, ArCH₃), 1.14 (t, 3H, ArCH₂CH₃)ppm.

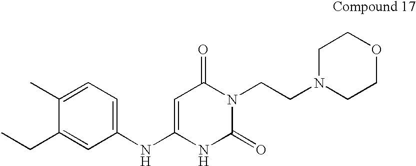

3-[2-(N-morpholino)ethyl]-6-(3-ethyl-4-methylanilino)uracil

Method II gave the product in 75% yield. 300 MHz ¹H NMR (DMSO-d₆): 10.48 (s, 1H, NH), 8.14 (S, 1H, NH), 6.90-7.18 (m, 3H, Ar—H), 4.72 (s, 1H, C₅—H), 3.82 (m, 2H, NCH₂), 3.47 (m, 4H, CH₂OCH₂), 3.24 (m, 2H, CH₂N), 2.58 (q, 2H, ArCH₂), 2.40 (m, 4H, CH₂NCH₂), 2.23 (s, 3H, ArCH₃), 1.14 (t, 3H, ArCH₂CH₃)ppm.

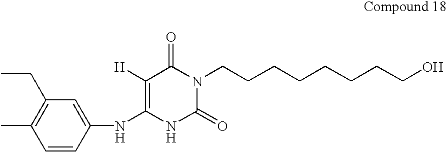

3-(8-Hydroxyoctyl)-6-(3-ethyl-4-methylanilino)uracil

Method II gave the product in 78% yield. 300 MHz ¹H NMR (DMSO-d₆): δ 1.14 (t, 3H, CH₃CH₂Ar), 1.20-1.30 (m, 8H, 4×CH₂), 1.37-1.52 (m, 4H, 2×CH₂), 2.21 (s, 3H, CH₃Ar), 2.57 (q, 2H, CH₂Ar), 3.35 (m, 2H, CH₂O), 3.64 (t, 2H, CH₂N), 4.30 (t, 1H, OH), 4.69 (s, 1H, C₅—H), 6.92-7.15 (m, 3H, Ar—H), 8.05 (s, 1H, NH), 10.35 (s, 1H, NH) ppm.

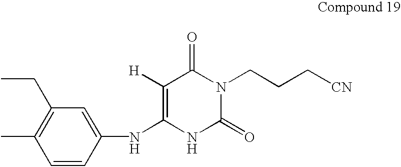

3-(3-Cyanopropyl)-6-(3-ethyl-4-methylanilino)uracil

Method II gave the product in 81% yield. 300 MHz ¹H NMR (DMSO-d₆): δ 1.14 (t, 3H, CH₃CH₂Ar), 1.79 (m, 2H, CH₂), 2.24 (s, 3H, CH₃Ar), 2.50 (t, 2H, CH₂CN), 2.57 (q, 2H, CH₂Ar), 3.79 (t, 2H, CH₂N), 4.74 (s, 1H, C₅—H), 6.92-7.15 (m, 3H, Ar—H), 8.12 (s, 1H, NH), 10.47 (s, 1H, NH) ppm.

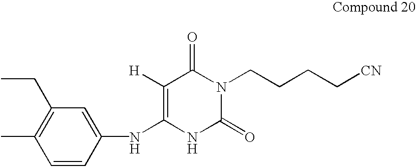

3-(4-Cyanobutyl)-6-(3-ethyl-4-methylanilino)uracil

Method II gave the product in 78% yield. 300 MHz ¹H NMR (DMSO-d₆): δ 1.14 (t, 3H, CH₃CH₂Ar), 1.50-1.61 (m, 4H, 2×CH₂), 2.24 (s, 3H, CH₃Ar), 2.58 (m, 4H, CH₂CN, CH₂Ar), 3.72(t, 2H, CH₂N), 4.73 (s, 1H, C₅—H), 6.92-7.15 (m, 3H, Ar—H), 8.12 (s, 1H, NH), 10.45 (s, 1H, NH) ppm.

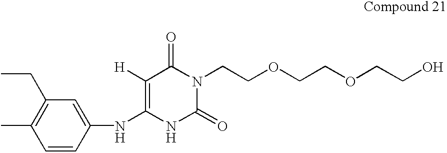

3-{2-[2-Hydroxyethoxy-(2-ethoxy)]ethyl}-6-(3-ethyl-4-methylanilino)uracil

Method II gave the product in 72% yield. 300 MHz ¹H NMR (DMSO-d₆): δ 1.14 (t, 3H, CH₃CH₂), 2.24 (s, 3H, CH₃Ar), 2.57 (q, 2H, CH₂Ar), 3.32-3.50 (m, 10H, 5×CH₂O), 3.87 (t, 2H, CH₂N), 4.56 (t, 1H, OH), 4.72 (s, 1H, C₅—H), 6.92-7.16 (m, 3H, Ar—H), 8.16 (s, 1H, NH), 10.50 (s, 1H, NH) ppm.

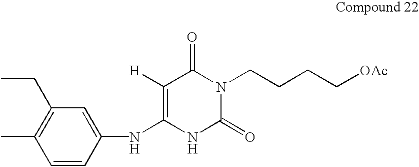

3-(4-Acetoxybutyl)-6-(3-ethyl-4-methylanilino)uracil

Method II gave the product in 84% yield. 300 MHz H NMR (DMSO-d₆): δ 1.14 (t, 3H, CH₃CH₂Ar), 1.53 (m, 4H, 2×CH₂), 2.0 (s, 3H, CH₃CO), 2.24 (s, 3H, CH₃Ar), 2.57 (q, 2H, CH₂Ar), 3.71 (t, 2H, CH₂O), 3.99 (t, 2H, CH₂N), 4.73 (s, 1H, C₅—H), 6.92-7.15 (m, 3H, Ar—H), 8.12 (s, 1H, NH), 10.43 (s, 1H, NH) ppm.

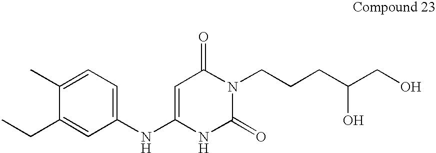

3-(4,5-Dihydroxypentyl)-6-(3-ethyl-4-methylanilino)uracil

Method II, starting with 6-amino-2-methoxy-3-[4,5-bis-(trimethylsiyloxy)pentyl]-4-pyrimidone, gave the product in 72% yield. 300 MHz ¹H NMR (DMSO-d₆): 10.42 (s, 1H, NH), 8.12 (S, 1H, NH), 6.90-7.13 (m, 3H, Ar—H), 4.72 (s, 1H, C₅—H), 4.41 (m, 2H, 2×OH), 3.66 (t, 2H, NCH₂), 3.20-3.36 (m, 3H, OCH₂, OCH), 2.58 (q, 2H, ArCH₂), 2.20 (s, 3H, ArCH₃), 1.33-1.68 (m, 4H, CH₂CH₂), 1.16 (t, 3H, ArCH₂CH₃) ppm.

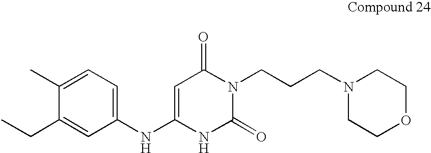

3-[3-(N-Morpholino)propyl]-6-(3-ethyl-4-methylanilino)uracil

Method II gave the product in 78% yield. 300 MHz ¹H NMR (DMSO-d₆): 10.40 (s, 1H, NH), 8.12 (S, 1H, NH), 6.95-7.18 (m, 3H, Ar—H), 4.75 (s, 1H, C₅—H), 3.78 (t, 2H, NCH₂), 3.59 (m, 4H, CH₂OCH₂), 2.60 (q, 2H, ArCH₂), 2.22-2.38 (m, 6H, NCH₂×3), 2.23 (s, 3H, ArCH₃), 1.68 (m, 2H, CH₂), 1.16 (t, 3H, ArCH₂CH₃)ppm.

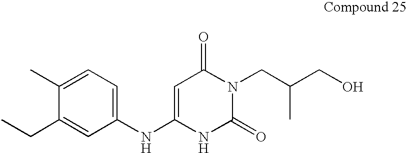

3-(3-Hydroxy-2-methylpropyl)-6-(3-ethyl-4-methylanilino)uracil

Method II gave the product in 60% yield. 300 MHz ¹H NMR (DMSO-d₆): 10.52 (s, 1H, NH), 8.19 (S, 1H, NH), 6.92-7.15 (m, 3H, Ar—H), 4.73 (s, 1H, C₅—H), 4.39 (t, 1H, OH), 3.61 (t, 2H, NCH₂), 3.33 (m, 2H, CH₂O), 2.59 (q, 2H, ArCH₂), 2.22 (s, 3H, ArCH₃), 1.96 (m, 1H, CH), 1.15 (t, 3H, ArCH₂CH₃), 0.88 (d, 3H, CH₃)ppm.

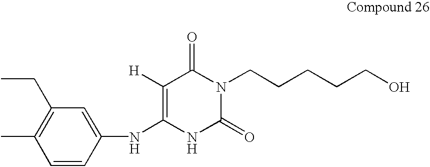

3-(5-Hydroxypentyl)-6-(3-ethyl-4-methylanilino)uracil

A solution of 1.0 M lithium aluminum hydride in tetrahydrofuran (1.5 ml) was added dropwise to a stirred solution of 3-[4-(ethoxycarbony)lbutyl]-6-(3-ethyl-4-methylanilino)uracil (160 mg, 0.43 mmol) in anhydrous tetrahydrofuran (30 ml) at room temperature. The reaction mixture was stirred at room temperature until disappearance of the stating material (20 minutes). Methanol (5 ml) was added dropwise to the solution, and the solvents were removed. Ethanol was added and the mixture was filtered and the solid washed carefully with ethanol. The solvent was removed, and the residue was purified by chromatography on silica gel with chloroform:methanol (98:2-96:4) as eluent, to give 141 mg (99% yield) of 3-(5-hydroxypentyl)-6-(3-ethyl-4-methylanilino)uracil. Crystallization from ethanol:water (1:1) gave white crystals. 300 MHz ¹H NMR (DMSO-d₆): δ 1.14 (t, 3H, CH₃CH₂Ar), 1.20-1.30 (m, 2H, CH₂), 1.37-1.52 (m, 4H, 2×CH₂), 2.24 (s, 3H, CH₃Ar), 2.57 (q, 2H, CH₂Ar), 3.34 (t, 2H, CH₂O), 3.67 (t, 2H, CH₂N), 4.35 (t, 1H, OH), 4.72 (s, 1H, C₅—H), 6.92-7.15 (m, 3H, Ar—H), 8.18 (s, 1H, NH), 10.48 (s, 1H, NH) ppm.

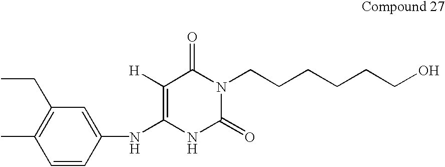

3-(6-Hydroxyhexyl)-6-(3-ethyl-4-methylanilino)uracil

Reduction of the corresponding ester by the method used for compound 26 gave the product in 94% yield. 300 MHz H NMR (DMSO-d₆): δ 1.14 (t, 3H, CH₃CH₂Ar), 1.17-1.48 (m, 8H, 4×CH₂), 2.24 (s, 3H, CH₃Ar), 2.57 (q, 2H, CH₂Ar), 3.34 (m, 2H, CH₂O), 3.67 (t, 2H, CH₂N), 4.35 (t, 1H, OH), 4.72 (s, 1H, C₅—H), 6.92-7.15 (m, 3H, Ar—H), 8.08 (s, 1H, NH), 10.39 (s, 1H, NH) ppm.

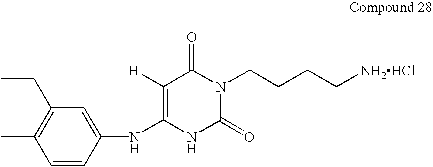

3-(4-Aminobutyl)-6-(3-ethyl-4-methylanilino)uracil Hydrochloride

Step 1. A solution of 0.5 M lithium aluminum hydride in diglyme (3 eq) was added dropwise to a stirred solution of 3-(3-cyanopropyl)-6-(3-ethyl-4-methylanilino)uracil (1 eq) in anhydrous diglyme at room temperature. The reaction mixture was stirred at room temperature until disappearance of the starting material. Methanol was added dropwise to the solution, and the solvents were removed. Ethanol was added and the mixture filtered, and the solid was washed carefully with ethanol. Ethanol was removed, and the residue was purified by chromatography on silica gel with chloroform:methanol as eluent, to give 3-(4-aminobutyl)-6-(3-ethyl-4-methylanilino)uracil (91% yield).

Step 2. 3-(4-Aminobutyl)-6-(3-ethyl-4-methylanilino)uracil was dissolved in chloroform and methanol, and a solution of 4.0 M hydrogen chloride in dioxane was added. The mixture was stirred at room temperature for 1 hour. The solvents were removed to give 3-(4-aminobutyl)-6-(3-ethyl-4-methylanilino)uracil hydrochloride as a white solid. 300 MHz ¹H NMR (DMSO-d₆): δ 1.11 (t, 3H, CH₃CH₂Ar), 1.50 (m, 4H, 2×CH₂), 2.21 (s, 3H, CH₃Ar), 2.57 (q, 2H, CH₂Ar), 2.78 (m, 2H, CH₂NH₂), 3.72 (t, 2H, CH₂N), 4.75 (s, 1H, C₅—H), 6.92-7.15 (m, 3H, Ar—H), 7.86 (br, 3H, NH₃), 8.89 (s, 1H, NH), 10.76 (s, 1H, NH) ppm.

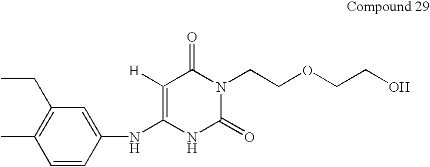

3-[2-(2-Hydroxyethoxy)ethyl]-6-(3-ethyl-4-methylanilino)uracil

A mixture of 3-[2-(2-benzyloxyethoxy)ethyl]-6-(3-ethyl-4-methylanilino)uracil (150 mg, 0.35 mmol) and 10% palladium on carbon (60 mg) in methanol (40 ml) was stirred under an atmosphere of hydrogen gas for 12 hours. The mixture was filtered, and the catalyst was washed with methanol. The combined filtrates were concentrated, and the residue was purified by chromatography on silica gel with chloroform:methanol (98:2-96:4) as eluent, to give 109 mg (92% yield) of 3-[2-(2-hydroxyethoxy)ethyl]-6-(3-ethyl-4-methylanilino)uracil. Crystallization from ethanol gave white crystals. 300 MHz ¹H NMR (DMSO-d₆): δ 1.14 (t, 3H, CH₃CH₂), 2.24 (s, 3H, CH₃Ar), 2.57 (q, 2H, CH₂Ar), 3.53-3.40 (m, 6H, 3×CH₂), 3.87 (t, 2H, CH₂N), 4.56 (t, 1H, OH), 4.72 (s, 1H, C₅—H), 6.92-7.16 (m, 3H, Ar—H), 8.16 (s, 1H, NH), 10.50 (s, 1H, NH) ppm.

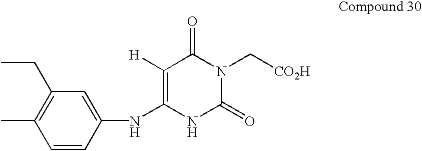

3-(Carboxymethyl)-6-(3-ethyl-4-methylanilino)uracil

A mixture of 3-(ethoxycarbonylmethyl)-6-(3-ethyl-4-methylanilino)uracil (20 mg, 0.06 mmol) and potassium hydroxide (5 mg, 0.09 mmol) in 5 ml of water and 5 ml of methanol was stirred at reflux for 5hours. The reaction mixture was cooled to room temperature and the solvents were removed. The residue was made acidic by addition of 4 ml of 6N hydrochloric acid. The precipitate was filtered and washed with water and dried to give 16 mg (87% yield) of 3-(carboxymethyl)-6-(3-ethyl-4-methylanilino)uracil as white crystals. 300 MHz ¹H NMR (DMSO-d₆): δ 1.14 (t, 3H, CH₃CH₂Ar), 2.24 (s, 3H, CH₃Ar), 2.58 (q, 2H, CH₂Ar), 4.34 (s, 2H, CH₂N), 4.73 (s, 1H, C₅—H), 6.94-7.17 (m, 3H, Ar—H), 8.20 (s, 1H, NH), 10.60 (s, 1H, NH), 12.79 (s, 1H, CO₂H) ppm.

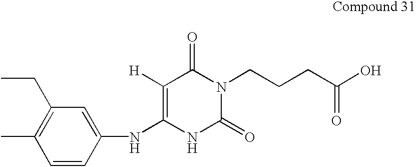

3-(3-Carboxypropyl)-6-(3-ethyl-4-methylanilino)uracil

A mixture of 3-[4-(ethoxycarbonyl)propyl]-6-(3-ethyl-4-methylanilino)uracil (80 mg, 0.22 mmol) and sodium hydroxide (10 mg, 0.25 mmol) in 10 ml of water and 10 ml of methanol was stirred at reflux for 3hours. The reaction mixture was cooled to room temperature and the solvents were removed. The residue was made acidic by addition of 8 ml of 6N hydrochloric acid, and the resulting precipitate was filtered and washed with water. Drying gave 68 mg (92% yield) of 3-(carboxylpropyl)-6-(3-ethyl-4-methylanilino)uracil as white crystals. 300 MHz ¹H NMR (DMSO-d₆): δ 1.14 (t, 3H, CH₃CH₂Ar), 1.17 (m, 2H, CH₂), 2.17 (t, 2H, CH₂CO₂H), 2.24 (s, 3H, CH₃Ar), 2.57 (q, 2H, CH₂Ar), 3.72 (t, 2H, CH₂N), 4.73 (s, 1H, C₅—H), 6.92-7.15 (m, 3H, Ar—H), 8.42 (s, 1H, NH), 10.80 (s, 1H, NH), 12.05 (s, 1H, CO₂H) ppm.

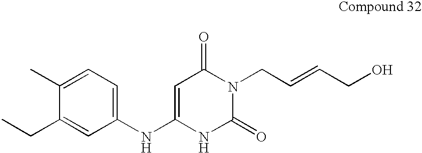

3-(4-Hydroxy-2-butenyl)-6-(3-ethyl-4-methylanilino)uracil

A solution of 1.0 M boron trichloride in methylene chloride (3.0 ml) was added dropwise to a stirred solution of 3-(4-benzyloxy-2-butenyl)-6-(3-ethyl-4-methylanilino)uracil (200 mg) in methylene chloride (8 ml) at −78° C. under nitrogen over 10 minutes. The reaction mixture was stirred at −78° C. for 5 hours, whereupon the reaction was quenched by a cautious addition of methanol (5 ml) and 10% ammonia in methanol (5 ml). The mixture was allowed to warm up to room temperature, and the insoluble portion was filtered off and washed with methylene chloride:methanol (4:1, 4×25 ml), The solvents were removed, and the residue was purified by chromatography on silica gel with methylene chloride:methanol (9:1-4:1) as eluent, to give 117.7 mg (72% yield) of 3-(4-hydroxy-2-butenyl)-6-(3-ethyl-4-methylanilino)uracil. 300 MHz ¹H NMR (DMSO-d₆): 10.46 (s, 1H, NH), 8.12 (S, 1H, NH), 6.93-7.13 (m, 3H, Ar—H), 5.60 (m, 1H, ═CH), 5.32 (m, 1H, ═CH), 4.72 (s, 1H, C₅—H), 4.66 (t, 1H, OH), 4.38 (m, 2H, OCH₂), 4.15 (m, 2H, NCH₂), 2.59 (q, 2H, ArCH₂), 2.22 (s, 3H, ArCH₃), 1.16 (t, 3H, ArCH₂CH₃)ppm.

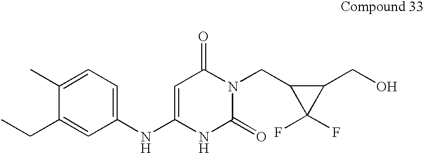

3-{[2-(Hydroxymethyl)-3,3-difluorocyclopropyl]methyl}-6-(3-ethyl-4-methylanilino)uracil

A solution of 1.0 M boron trichloride in methylene chloride (3.0 ml) was added dropwise to a stirred solution of 3-{[2-(benzyloxymethyl)-3,3-difluorocyclopropyl]methyl}-6-(3-ethyl-4-methylanilino)uracil (220 mg) in methylele chloride (8 ml) at −78° C. under nitrogen over 10 minutes. The reaction mixture was stirred at −78° C. for 5 hours, whereupon the reaction was quenched by a cautious addition of methanol (5 ml) and 10% ammonia in methanol (5 ml). The mixture was allowed to warm up to room temperature, the insoluble portion filtered off and washed with methylene chloride (25 ml), and the combined filtrates were evaporated. The residue was purified by chromatography on silica gel with methylene chloride:methanol as eluent, to give 148 mg (89% yield) of 3-{[2-(hydroxymethyl)-3,3-difluorocyclopropyl]methyl}-6-(3-ethyl-4-methylanilino)uracil. 300 MHz ¹H NMR (DMSO-d₆): 10.48 (s, 1H, NH), 8.13 (S, 1H, NH), 6.93-7.13 (m, 3H, Ar—H), 4.82 (t, 1H, OH), 4.73 (s, 1H, C₅—H), 3.82 (m, 2H, OCH₂), 3.58 (m, 2H, NCH₂), 2.59 (q, 2H, ArCH₂), 2.22 (s, 3H, ArCH₃), 2.10 (m, 1H, CF₂H), 1.90 (m, 1H, CF₂H), 1.16 (t, 3H, ArCH₂CH₃)ppm.

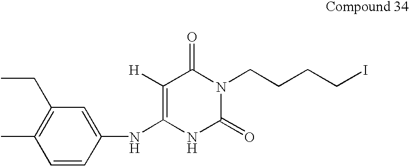

3-(4-Iodobutyl)-6-(3-ethyl-4-methylanilino)uracil

Trimethylsilyl iodide (2-5 eq) was added to a stirred solution of 3-(4-methoxybutyl)-6-(3-ethyl-4-methylanilino)uracil [or 3-(4-hydroxybutyl)-6-(3-ethyl-4-methylanilino)uracil, or 3-(4-acetoxybutyl)-6-(3-ethyl-4-methylanilino)uracil] (1 eq) in dry chloroform. The reaction mixture was stirred at reflux for 3 hours-2 days, until disappearance of starting material. Methanol and sodium sulfite were then added to the brown-purple solution. After stirring at room temperature for 10 minutes, the mixture was filtered and the solvent was removed. The residue was purified by chromatography on silica gel with chloroform:methanol (2:98-7:93) as eluent to give 3-(4-iodobutyl)-6-(3-ethyl-4-methylanilino)uracil in high yield (80-95%). 300 MHz ¹H NMR (DMSO-d₆): δ 1.14 (t, 3H, CH₃CH₂Ar), 1.54-1.78 (m, 4H, 2×CH₂), 2.24 (s, 3H, CH₃Ar), 2.57 (q, 2H, CH₂Ar), 3.29 (t, 2H, CH₂I), 3.72 (t, 2H, CH₂N), 4.73 (s, 1H, C₅—H), 6.92-7.15 (m, 3H, Ar—H), 8.15 (s, 1H, NH), 10.45 (s, 1H, NH) ppm.

General Method for the Reaction of 3-(4-iodobutyl)-6-(3-ethyl-4-methylanilino)uracil with Nucleophiles

Method IIIa:

A mixture of 3-(4-iodobutyl)-6-(3-ethyl-4-methylanilino)uracil, potassium carbonate and nucleophile in a solvent (acetone, acetonitrile or N,N-dimethylformamide) was stirred at room temperature. Once the reaction was complete, as monitored by thin layer chromatography, the solution was concentrated in vacuo, and water was added. The mixture was extracted with chloroform, and the extracts were dried over sodium sulfate. After removal of chloroform, the residue was purified by chromatography on silica gel using chloroform:methanol eluent to give the product.

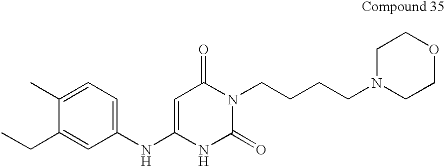

3-[4-(N-morpholino)butyl]-6-(3-ethyl-4-methylanilino)uracil

Method IIIa gave the product in 72% yield. 300 MHz ¹H NMR (DMSO-d₆): 10.38 (s, 1H, NH), 8.04 (S, 1H, NH), 6.90-7.14 (m, 3H, Ar—H), 4.72 (s, 1H, C₅—H), 3.68 (m, 2H, NCH₂), 3.56 (m, 4H, CH₂OCH₂), 3.30 (m, 2H, CH₂N), 2.55 (q, 2H, ArCH₂), 2.20-2.30 (m, 7H, CH₂NCH₂, ArCH₃), 1.30-1.52 (m, 4H, CH₂CH₂), 1.12 (t, 3H, ArCH₂CH₃) ppm.

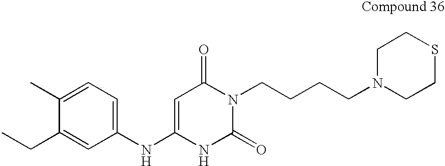

3-[(N-Thiomorpholino)butyl]-6-(3-ethyl-4-methylanilino)uracil

Method IIIa gave the product in 77% yield. 300 MHz ¹H NMR (DMSO-d₆): 10.03 (s, 1H, NH), 8.10 (S, 1H, NH), 6.90-7.12 (m, 3H, Ar—H), 4.71 (s, 1H, C₅—H), 3.62 (m, 2H, NCH₂), 3.30 (m, 2H, CH₂N), 2.55 (q, 2H, ArCH₂), 2.45 (m, 8H, NCH₂CH₂S×2), 2.18 (s, 3H, ArCH₃), 1.30-1.51 (m, 4H, CH₂CH₂), 1.10 (t, 3H, ArCH₂CH₃) ppm.

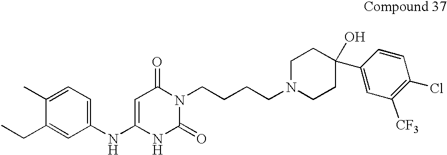

3{[4-(3-Trifluoromethylphenyl-4-chloro-)-4-hydroxypiperidino]butyl}-6-(3-ethyl-4-methylanilino)uracil

Method IIIa gave the product in 54% yield. 300 MHz ¹H NMR (DMSO-d₆): 10.36 (s, 1H, NH), 8.14 (S, 1H, NH), 7.68-7.90 (m, 3H, Ar—H), 6.90-7.14 (m, 3H, Ar—H), 5.11 (s, 1H, OH), 4.72 (s, 1H, C₅—H), 3.68 (m, 2H, NCH₂), 3.40 (m, 2H, CH₂N), 2.52 (q, 2H, ArCH₂), 2.24-2.38 (m, 4H, CH₂NCH₂), 2.10 (s, 3H, ArCH₃), 1.88(t, 2H, CH₂), 1.50 (m, 6H, CH₂×3), 1.10 (t, 3H, ArCH₂CH₃) ppm.

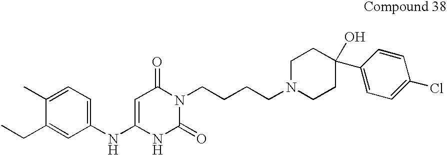

3{[4-(4-Chlorophenyl)-4-hydroxypiperidino] butyl}-6-(3-ethyl-4-methylanilino)uracil

Method IIIa gave the product in 64% yield. 300 MHz ¹H NMR (DMSO-d₆): 10.35 (s, 1H, NH), 8.09 (S, 1H, NH), 7.50 (d, 2H, Ar—H), 7.31 (d, 2H, Ar—H), 6.88-7.12 (m, 3H, Ar—H), 4.90 (s, 1H, OH), 4.72 (s, 1H, C₅—H), 3.62 (m, 2H, NCH₂), 3.42 (m, 2H, CH₂N), 2.52 (q, 2H, ArCH₂), 2.24-2.60 (m, 4H, CH₂NCH₂), 2.10 (s, 3H, ArCH₃), 1.88(t, 2H, CH₂), 1.52 (m, 6H, CH₂×3), 1.10 (t, 3H, ArCH₂CH₃) ppm.

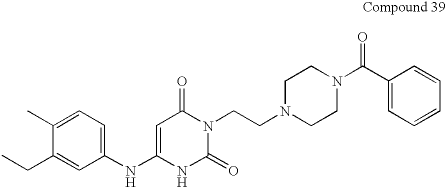

3-[2-(4-Benzoylpiperazino)ethyl]-6-(3-ethyl-4-methylanilino)uracil

Method IIIa gave the product in 81% yield. 300 MHz ¹H NMR (DMSO-d₆): 10.45 (s, 1H, NH), 8.12 (S, 1H, NH), 6.92-7.45 (m, 8H, Ar—H), 4.72 (s, 1H, C₅—H), 3.83 (t, 2H, NCH₂), 3.59 (m, 2H, CH₂N), 3.30 (m, 4H, NCH₂×2), 2.42-2.60 (m, 6H, NCH₂×2, ArCH₂), 2.24 (s, 3H, ArCH₃), 1.16 (t, 3H, ArCH₂CH₃) ppm.

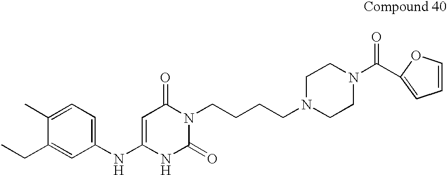

3-{4-[4-(2-Furoyl)piperazino]butyl}-6-(3-ethyl-4-methylanilino)uracil

Method IIIa gave the product in 68% yield. 300 MHz ¹H NMR (DMSO-d₆): 10.42 (s, 1H, NH), 8.12 (S, 1H, NH), 7.83 (s, 1H, furyl-H), 7.15 (m, 1H, Ar—H), 6.96 (m, 3H, 2×Ar—H, furyl-H), 6.62 (s, 1H, furyl-H), 4.73 (s, 1H, C₅—H), 3.68 (m, 4H, 2×NCH₂), 3.33 (m, 4H, 2×CH₂N), 2.58 (q, 2H, ArCH₂), 2.30-2.45 (m, 4H, 2×NCH₂), 2.24 (s, 3H, ArCH₃), 1.40-1.50 (m, 4H, CH₂CH₂), 1.15 (t, 3H, ArCH₂CH₃) ppm.

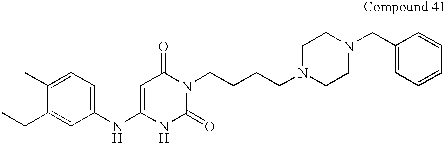

3-[4-(4-Benzylpiperazino)butyl]-6-(3-ethyl-4-methylanilino)uracil

Method IIIa gave the product in 78% yield. 300 MHz ¹H NMR (DMSO-d₆): 10.38 (s, 1H, NH), 8.15 (S, 1H, NH), 6.88-7.32 (m, 8H, Ar—H), 4.70 (s, 1H, C₅—H), 3.64 (t, 2H, NCH₂), 3.40 (m, 2H, NCH₂), 3.28 (m, 4H, 2×CH₂N), 2.52 (q, 2H, ArCH₂), 2.16-2.38 (m, 9H, 3×NCH₂, Ar—CH₃), 1.26-1.50 (m, 4H, CH₂CH₂), 1.10 (t, 3H, ArCH₂CH₃) ppm.

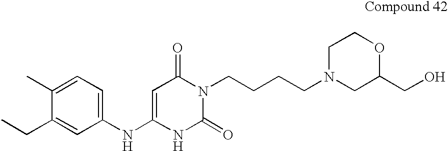

3-{1[4-(3-Hydoxymethyl)morpholino]butyl}-6-(3-ethyl-4-methylanilino)uracil

Method IIIa gave the product in 83% yield. 300 MHz ¹H NMR (DMSO-d₆): 10.35 (s, 1H, NH), 8.08 (S, 1H, NH), 6.88-7.10 (m, 3H, Ar—H), 4.72 (s, 1H, C5-H), 4.58 (t, 1H, OH), 3.70 (m, 4H, NCH₂, CH₂OH), 3.30-3.45(m, 3H, OCH₂, OCH), 2.78 (d, 1H, NCH), 2.60 (m, 3H, NCH, ArCH₂), 2.20 (m, 5H, NCH₂, Ar—CH₃), 1.90 (t, 1H, NCH), 1.68 (t, 1H, NCH), 1.33-1.50 (m, 4H, CH₂CH₂), 1.12 (t, 3H, ArCH₂CH₃) ppm.

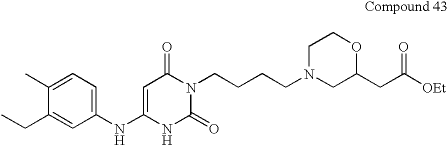

3-{[4-(3-Ethoxycarbonylmethyl)morpholino]butyl}-6-(3-ethyl-4-methylanilino)uracil

Method IIIa gave the product in 56% yield. 300 MHz ¹H NMR (DMSO-d₆): 10.42 (s, 1H, NH), 8.04 (S, 1H, NH), 6.92-7.13 (m, 3H, Ar—H), 4.72 (s, 1H, C₅—H), 4.06 (q, 2H, CO₂CH₂), 3.68 (m, 5H, NCH₂, OCH₂, OCH), 2.70 (d, 1H, NCH), 2.30-2.62 (m, 7H, CH₂N, CH₂CO₂, NCH, ArCH₂), 2.20 (s, 3H, Ar—CH₃), 1.82 (t, 1H, NCH), 1.68 (t, 1H, NCH), 1.30-1.52 (m, 4H, CH₂CH₂), 1.15 (m, 6H, ArCH₂CH₃, CO₂CH₂CH₃) ppm.

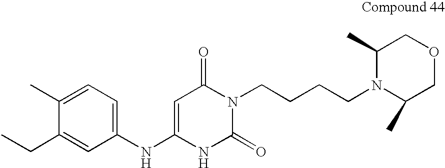

3-[4-(C is-2,6-dimethylmorpholino)butyl]-6-(3-ethyl-4-methylanilino)uracil

Method IIIa gave the product in 67% yield. 300 MHz ¹H NMR (DMSO-d₆): 10.38 (s, 1H, NH), 8.06 (S, 1H, NH), 6.92-7.13 (m, 3H, Ar—H), 4.72 (s, 1H, C₅—H), 3.82 (m, 1H, OCH), 3.68 (t, 2H, NCH₂), 3.50 (m, 1H, OCH), 2.64 (d, 1H, OCH), 2.59 (q, 2H, ArCH₂), 2.32 (d, 1H, OCH), 2.19 (m, 6H, CH₂N, NCH, Ar—CH₃), 2.00 (m, 1H, NCH), 1.30-1.52 (m, 4H, CH₂CH₂), 1.16 (m, 6H, 2×CH₃), 0.98 (m, 3H, CH₃) ppm.

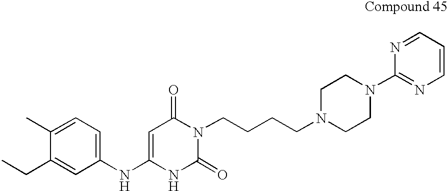

3-{4-[4-(2-pyrimidinyl)piperazinyl] butyl}-6-(3-ethyl-4-methyylanilino)uracil

Method IIIa gave the product in 62% yield. 300 MHz ¹H NMR (DMSO-d₆): 10.41 (s, 1H, NH), 8.38 (m, 2H, pyrimidine-H), 8.12 (S, 1H, NH), 6.93-7.15 (m, 3H, Ar—H), 6.61 (t, 1H, pyrimidine-H), 4.73 (s, 1H, C₅—H), 3.70 (m, 6H, NCH₂×3), 3.32 (m, 2H, NCH₂), 2.60 (q, 2H, ArCH₂), 2.39 (m, 4H, CH₂N×2), 2.22 (s, 3H, Ar—CH₃), 1.45-1.51 (m, 4H, CH₂CH₂), 1.14 (t, 3H, ArCH₂CH₃)ppm.

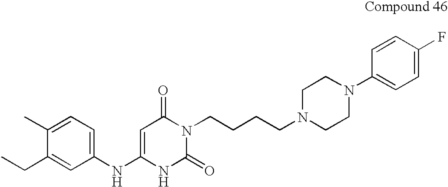

3-{4-[(4-fluorophenyl)piperazinyl]butyl}-6-(3-ethyl-4-methyylanilino)uracil

Method IIIa gave the product in 57% yield. 300 MHz ¹H NMR (DMSO-d₆): 10.41 (s, 1H, NH), 8.11 (S, 1H, NH), 6.93-7.15 (m, 7H, Ar—H), 4.73 (s, 1H, C₅—H), 3.71 (t, 2H, NCH₂), 3.30 (m, 2H, NCH₂), 2.99 (m, 4H, NCH₂×2), 2.59 (q, 2H, ArCH₂), 2.55 (m, 2H, NCH₂), 2.33 (m, 2H, CH₂N), 2.21 (s, 3H, Ar—CH₃), 1.42-1.54 (m, 4H, CH₂CH₂), 1.14 (t, 3H, ArCH₂CH₃)ppm.

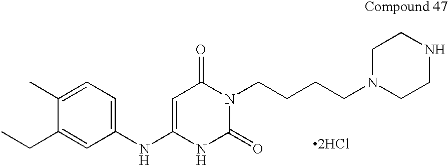

3-(4-Piperazinobutyl]-6-(3-ethyl-4-methylanilino)uracil Dihydrochloride

Step 1. Method IIIa with N-Boc-piperazine gave 3-{4-[4-(t-butoxycarbonyl)-piperazino]butyl}-6-(3-ethyl-4-methylanilino)uracil in 62% yield. 300 MHz ¹H NMR (DMSO-d₆): 10.40 (s, 1H, NH), 8.10 (s, 1H, NH), 6.93-7.15 (m, 3H, Ar—H), 4.72 (s, 1H, C5-H), 3.70 (t, 2H, NCH₂), 3.20 (m, 4H, 2×CH₂N), 2.60 (q, 2H, ArCH₂), 2.28 (m, 6H, 3×CH₂N), 2.26 (s, 3H, ArCH₃), 1.48-1.55 (m, 4H, CH₂CH₂), 1.40 (s, 9H, t-Bu), 1.16 (t, 3H, ArCH₂CH₃) ppm.

Step 2. A solution of the Boc-protected product (2.4 g) in CHCl₃:MeOH, 3:1 (20 ml) was treated with 50 ml of 4.0M HCl in dioxane. The mixture was stirred at room temperature for 4 hours. After removal of the solvent, the residue was washed with diethyl ether and dried in vacuo to give the product as a colorless solid in 94% yield. 300 MHz ¹H NMR (DMSO-d₆): 10.58 (s, 1H, NH), 10.38 (s, 3H, NH₃Cl), 8.50 (S, 1H, NH), 6.93-7.15 (m, 3H, Ar—H), 4.72 (s, 1H, C5-H), 3.72 (t, 2H, NCH₂), 3.50 (m, 6H, 3×CH₂N), 3.18 (m, 4H, 2×CH₂N), 2.60 (q, 2H, ArCH₂), 2.22 (s, 3H, ArCH₃), 1.50-1.70 (m, 4H, CH₂CH₂), 1.16 (t, 3H, ArCH₂CH₃) ppm.

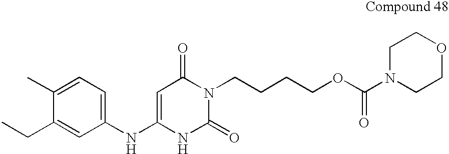

3-[4-(N-morpholinocarbonyloxy)butyl]-6-(3-ethyl-4-methylanilino)uracil

Method IIIa gave the product in 82% yield. 300 MHz ¹H NMR (DMSO-d₆): 10.42 (s, 1H, NH), 8.10 (S, 1H, NH), 6.93-7.15 (m, 3H, Ar—H), 4.72 (s, 1H, C₅—H), 4.05 (t, 2H, CO₂CH₂), 3.68 (t, 2H, NCH₂), 3.48 (m, 4H, CH₂OCH₂), 3.32 (m, 4H, NCH₂×2), 2.60 (q, 2H, ArCH₂), 2.22 (s, 3H, ArCH₃), 1.50 (m, 4H, CH₂CH₂), 1.16 (t, 3H, ArCH₂CH₃) ppm.

General Method for the Reaction of 3-(4-iodoalkyl)-6-(3-ethyl-4-methylanilino)uracil with Nucleophiles

Method IIIb:

3-(4-iodopentyl)-6-(3-ethyl-4-methylanilino)uracil or 3-(4-iodobutyl)-6-(3-ethyl-4-methylanilino)uracil (1 eq) was dissolved in dry DMF, then nucleophile (1-1.2 eq) and sodium carbonate or potassium carbonate (1.2-2 eq) were added to the mixture which was stirred at room temperature (or 60° C.) from 15 h to 2 days. The solvent was evaporated in vacuum, the residue dissolved in a mixture of dichloromethane:methanol and washed with H₂O. Organic fraction was dried over sodium sulfate, evaporated to dryness and separated by column chromatography on silica gel using dichloromethane:methanol as eluent (or triturated with acetonitrile and ether) to give target compounds.

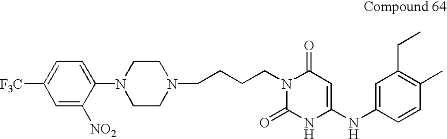

3-{4-[4-(2-nitro-4-trifluoromethylphenyl)-1-piperazinyl]butyl}-6-(3-ethyl-4-methylanilino)uracil

Method IIIb gave the compound 64 in 16% yield. 400 MHz ¹H NMR (CDCl₃): 9.67 (s, 1H, NH), 8.06 (s, 1H, ArH), 7.67 (d, 1H, ArH), 7.05-7.21 (m, 4H, ArH), 4.24 (m, 5H, C₅—H and 2×NCH₂ₚᵢₚ), 3.69 (m, 2H, NCH₂), 3.22 (m, 4H, 2×NCH_{2 pip}), 2.72 (m, 2H, NCH₂), 2.63 (q, 2H, ArCH₂CH₃), 2.24 (s, 3H, ArCH₃), 2.02 (m, 2H, CH₂), 1.81 (m, 2H, CH₂), 1.2 (t, 3H, ArCH₂CH₃) ppm.

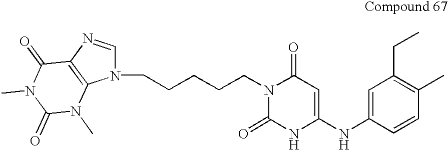

3-{5-[9-(1,3-dimethyl-2,6-dioxopurinyl)]pentyl}-6-(3-ethyl-4-methylanilino)uracil

Theophylline (27 mg, 0.15 mmol) was dissolved in dry DMF (5 ml), sodium hydride (4 mg, 0.15 mmol) was added to this solution and the mixture was stirred for 30 minutes under nitrogen at room temperature. A solution of 3-(4-iodopentyl)-6-(3-ethyl-4-methylanilino)uracil (59 mg, 0.13 mmol) in dry DMF was added dropwise and the mixture was stirred under nitrogen at room temperature overnight. Then, the solvent was evaporated to dryness and the residue was purified by column chromatography on silica gel using dichloromethane:methanol (95:5) as eluent to give 19 mg (22% yield) of product as a white solid (complex with theophylline 1:1). 400 MHz ¹H NMR (DMSO-d₆): 10.35 (s, 1H, NH), 8.04 (s, 1H, purine-H), 7.1 (d, 1H, ArH), 6.9-7.12 (m, 2H, ArH), 4.68 (s, 1H, C5-H), 4.2 (t, 2H, NCH₂), 3.62 (t, 2H, NCH₂), 3.19 (s, 3H, NCH_{3purine),} 3.21 (s, 3H, NCH₃ₚᵤᵣᵢₙₑ), 2.55 (q, 2H, ArCH₂CH₃), 2.19 (s, 3H, ArCH₃), 1.75 (m, 2H, CH₂), 1.46 (m, 2H, CH₂), 1.18 (m, 2H, CH₂), 1.13 (t, 3H, ArCH₂CH₃) ppm.

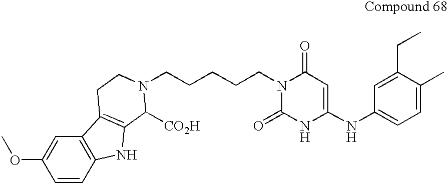

3-[5-(1-carboxy-6-methoxy-2,3,4,9-tetrahydro-1H-β-carboline-2-yl)pentyl]-6-(3-ethyl-4-methylanilino)uracil

Method IIIb gave the compound 68 in 97% yield. 400 MHz ¹H NMR (CD₃OD): 8.84 (s, 1H, NH), 6.77-7.14 (m, 6H, ArH), 4.87 (s, 1H, C5-H), 4.27 (s, 1H, NCH), 4.10 (t, 2H, NCH₂), 3.95 (m, 2H, NCH₂), 3.79 (s, 3H, OCH₃), 3.38 (m, 2H, NCH₂), 2.99-3.09 (m, 2H, CH₂), 2.61 (q, 2H, ArCH₂CH₃), 2.28 (s, 3H, ArCH₃), 1.91 (m, 2H, CH₂), 1.61-1.72 (m, 2H, CH₂), 1.41 (m, 2H, CH₂), 1.19 (t, 3H, ArCH₂CH₃) ppm.

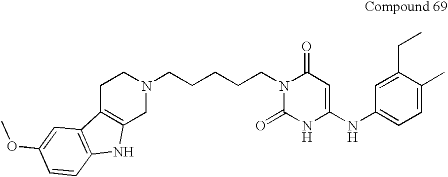

3-[5-(6-methoxy-2,3,4,9-tetrahydro-1H-α-carboline-2-yl)pentyl]-6-(3-ethyl-4-methylanilino)uracil

Method IIIb gave the compound 69 in 36% yield. 400 MHz ¹H NMR (CD₃OD): 7.15-7.2 (m, 2H, ArH), 6.93-7.0 (m, 3H, ArH), 6.74 (dd, 1H, ArH), 4.88 (s, 1H, C5-H), 4.13 (m, 2H, NCH₂), 3.87 (m, 2H, NCH₂), 3.79 (s, 3H, OCH₃), 3.31 (m, 2H, NCH₂), 2.94-3.03 (m, 4H, NCH₂ and CH₂), 2.63 (q, 2H, ArCH₂CH₃), 2.29 (s, 3H, ArCH₃), 1.81 (m, 2H, CH₂), 1.69 (m, 2H, CH₂), 1.42 (m, 2H, CH₂), 1.20 (t, 3H, ArCH₂CH₃) ppm.

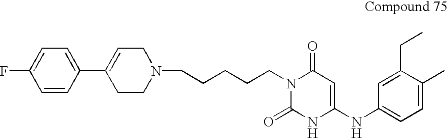

3-{5-1-(4-(4-fluorophenyl)-1,2,3,6-tetrahydropyridinyl)]pentyl}-6-(3-ethyl-4-methylanilino)uracil

Method IIIb afforded the compound 75 in 76% yield after column purification. 400 MHz ¹H NMR (DMSO-d₆): 9.44 (s, 1H, NH), 8.07 (s, 1H, NH), 7.49-7.51 (m, 2H, ArH), 7.17-7.21 (m, 2H, ArH), 7.13 (d, 1H, ArH), 6.90-6.94 (m, 2H, ArH), 6.13 (s, 1H, CH), 4.69 (s, 1H, C5-H), 3.95 (m, 1H, CHN), 3.66-3.7 (m, 4H, 2×NCH₂), 3.12-3.25 (m, 3H, CHN and NCH₂), 2.63-2.72 (m, 2H, CH₂), 2.53 (q, 2H, ArCH₂CH₃), 2.2 (s, 3H, ArCH₃), 1.67 (m, 2H, CH₂), 1.49-1.55 (m, 2H, CH₂), 1.25-1.3 (m, 2H, CH₂), 1.1 (t, 3H, ArCH₂CH₃).

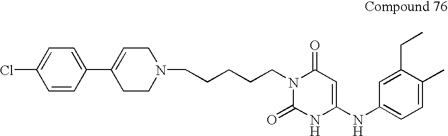

3-{5-[1-(4-(4-chlorophenyl)-1,2,3,6-tetrahydropyridinyl)]pentyl}-6-(3-ethyl-4-methylanilino)uracil

Method IIIb gave the compound 76 in 33% yield after column purification. 400 MHz ¹H NMR (CD₃OD): 7.42 (m, 2H, ArH), 7.32 (m, 2H, ArH), 7.15 (d, 1H, ArH), 7.0 (d, 1H, ArH), 6.93 (dd, 1H, ArH), 6.14 (m, 1H, CH), 4.88 (s, 1H, C5-H), 3.85 (t, 2H, NCH₂), 3.46 (m, 2H, NCH₂), 3.05 (t, 2H, NCH₂), 2.77 (m, 2H, NCH₂), 2.67 (m, 2H, CH₂), 2.63 (q, 2H, ArCH₂CH₃), 2.28 (s, 3H, ArCH₃), 1.65-1.73 (m, 4H, 2×CH₂), 1.39 (m, 2H, CH₂), 1.19 (t, 3H, ArCH₂CH₃).

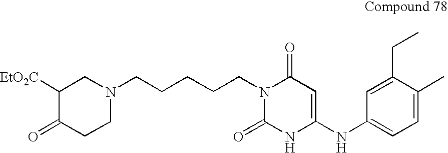

3-{5-(3-ethoxycarbonyl-4-oxopiperidinyl)pentyl}-6-(3-ethyl-4-methylanilino)uracil

Method IIIb gave the compound 78 in 37% yield after a following work-up: DMF was evaporated to dryness, the residue dissolved in dichloromethane:methanol and washed with H₂O (back-extracted with dichloromethane:methanol). Organic fraction was dried over sodium sulfate, concentrated and purified by column chromatography on silica gel using dichloromethane:methanol as eluent. 400 MHz ¹H NMR (CD₃OD): 7.16 (d, 1H, Ar—H), 7.01 (d, 1H, Ar—H), 6.94 (dd, 1H, Ar—H), 4.82 (s, 1H, C5-H), 3.88-3.61 (m, 6H, 3×NCH₂), 3.28 (s, 3H,OMe), 3.22 (t, 2H, CH₂N), 2.85 (t, 1H, CH), 2.63 (q, 2H, ArCH₂CH₃), 2.28 (s, 3H, ArCH₃), 1.63 (m, 4H, 2×CH₂), 1.38 (m, 2H, CH₂), 1.21 (t, 3H, ArCH₂CH₃) ppm.

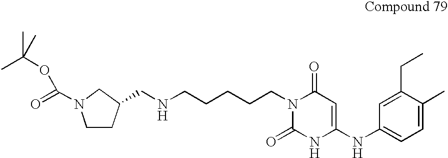

3(S)-{5-[(N-tert-butoxycarbonylpyrrolidin-3-yl)methylamino]pentyl}-6-(3-ethyl-4-methylanilino)uracil

Method IIIb gave the compound 79 in 84% yield. 400 MHz ¹H NMR (CD₃OD): 7.15 (m, 2H, ArH), 7.01 (m, 1H, ArH), 6.94 (m, 1H, ArH), 4.9 (s, 1H, C5-H), 3.85 (t, 2H, NCH₂), 3.62 (m, 1H, NCH), 3.46 (m, 1H, NCH), 3.30 (m, 2H, NCH₂), 2.99-3.17 (m, 4H, 2×NCH₂), 2.56-2.66 (m, 3H, ArCH₂CH₃ and CH), 2.29 (s, 3H, ArCH₃), 2.14 (m, 1H, CH), 1.62-1.80 (m, 5H, 2×CH₂ and CH), 1.39-1.45 (m, 1H, CH₂ and OtBu), 1.19 (t, 3H, ArCH₂CH₃).

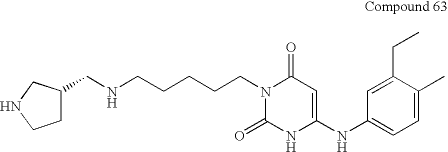

3(S)-{5-[(3-pyrrolidinyl)methylamino]pentyl}-6-(3-ethyl-4methylanilino)uracil

Compound 79 (46 mg, 0.09 mmol) was dissolved in trifluoroacetic acid (0.2 ml), the mixture was stirred at room temperature for 2 hours, evaporated under reduced pressure and purified by flash chromatography on silica gel using EtOH: H₂O: NH₄OH as eluent (gradient from 98:1:1 to 80:10:10) to give 33.2 mg (yield 60%) of compound 63. 400 MHz ¹H NMR (CF₃COOD): 8.1 (s, 1H, NH), 7.66 (m, 1H, ArH), 7.33-7.42 (m, 2H, ArH), 4.27-4.47 (m, 4H, CH₂N, C5-H and CHN), 4.12 (m, 1H, CHN), 3.92-3.99 (m, 1H, CHN), 3.83 (m, 3H, CH and NCH₂), 3.65 (m, 2H, NCH₂), 3.46 (m, 1H, CHN), 3.06 (q, 2H, ArCH₂CH₃), 2.88 (m, 1H, CHN), 2.72 (s, 3H, ArCH₃), 2.33-2.43 (m, 1H, CHN), 2.19-2.25 (m, 4H, 2×CH₂), 1.88 (m, 2H, CH₂), 1.60 (t, 3H, ArCH₂CH₃).

Reaction of 3-(4-aminoalkyl)-6-(3-ethyl-4-methylanilino)uracil with Carbonyl Chlorides, Aryl Halide and Alkyl Halide:

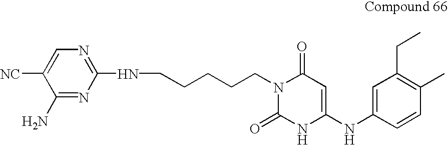

3-{5-[(4-amino-5-cyano-2-pyrimidinyl)amino]pentyl}-6-(3-ethyl-4-methylanilino)uracil

To a suspension of 3-(5-aminopentyl)-6-anilinouracil hydrochloride (61 mg, 0.166 mmol) and 4-amino-2-chloro-pyrimidine-5-carbonitrile (26 mg, 0.166 mmol) in acetonitrile (10 ml) was added sodium carbonate (35 mg, 0.332 mmol). The mixture was refluxed overnight, filtered, solvent evaporated and the residue separated by column chromatography on silica gel using dichloromethane:methanol (gradient from 100:0 to 90:10) as eluent affording 17 mg (23%) of compound 66. 400 MHz ¹H NMR (CD₃OD): 8.65 (s, 1H, Ar_{pyr}H), 8.40 (d 2H, NH₂), 8.04 (s, 1H, NH), 7.40 (d, 1H, ArH), 7.07 (m, 1H, ArH), 6.85 (m, 1H, ArH), 4.83 (s, 1H, C5-H), 3.92 (m, 2H, NCH₂), 3.34 (m, 2H, NCH₂), 2.59 (q, 2H, ArCH₂CH₃), 2.24 (s, 3H, ArCH₃), 1.62-1.70 (m, 4H, 2×CH₂), 1.39 (m, 2H, CH₂), 1.20 (t, 3H, ArCH₂CH₃) ppm.

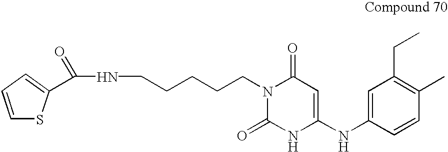

3-[5-(2-thiophenecarboxamido)pentyl]-6-(3-ethyl-4-methylanilino)uracil

To a solution of 3-(5-aminopentyl)-6-anilinouracil hydrochloride (32 mg, 0.22 mmol) in dry pyridine (2 ml) was added thiophene-2-carbonyl chloride (54 mg, 0.14 mmol). The mixture was stirred for 4 hours at room temperature, evaporated to dryness and the residue was triturated with acetonitrile. Precipitate was filtered off and filtrate was concentrated and purified by column chromatography on silica gel using dichloromethane:methanol (gradient from 99:1 to 98:2) as eluent to give 40 mg (yield 64%) of compound 70. 400 MHz ¹H NMR (CD₃OD): 8.4 (s, 1H, NH), 7.65 (dd, 1H, ArₜₕᵢₒH), 7.6 (dd, 1H, ArₜₕᵢₒH), 7.15 (d, 1H, ArH), 7.08 (dd, 1H, ArₜₕᵢₒH), 6.99 (d, 1H, ArH), 6.92 (dd, 1H, ArH), 4.90 (s, 1H, C5-H), 3.84 (t, 2H, NCH₂), 3.33 (m, 2H, NCH₂), 2.63 (q,2H,ArCH₂CH₃), 2.29 (s, 3H, ArCH₃), 1.60-1.66 (m, 4H, 2×CH₂), 1.36-1.42 (m, 2H, CH₂), 1.18 (t, 3H, ArCH₂CH₃).

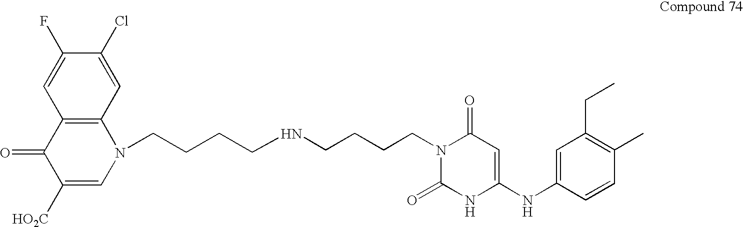

3-{4-[4-(3-carboxy-4-oxo-6-fluoro-7-chloroquinoline-1-yl)butylamino]butyl}-6-(3-ethyl-4-methylanilino)uracil

A mixture of 7-chloro-6-fluoro-4-oxo-1,4-dihydro-quinoline-3-carboxylic acid ethyl ester (860 mg, 3.18 mmol), 1,4-diiodobutane (4.94 g, 16 mmol), potassium carbonate (4.2 g, 30.38 mmol) and tetrabutylammonium bromide (340 mg, 1.05 mmol) was stirred at room temperature overnight. The solvent was removed and the residue was purified by flash chromatography on silica gel using ethyl acetate (100%) as eluent to obtain 757 mg (yield 69%) of 7-chloro-6-fluoro-1-(4-iodo-butyl)-4-oxo-1,4-dihydro-quinoline-3-carboxylic acid ethyl ester.

7-chloro-6-fluoro-1-(4-amino-butyl)-4-oxo-1,4-dihydro-quinoline-3-carboxylic acid ethyl ester (757 mg, 1.68 mmol), 3-(4-iodobutyl)-6-(3-ethyl-4-methylanilino)uracil (600 mg, 1.70 mmol), potassium carbonate (700 mg, 5.06 mmol) and tetrabutylammonium bromide (160 mg, 0.5 mmol) were stirred at room temperature overnight. The solvent was removed and the residue was purified by preparative HPLC (35% to 65% acetonitrile:H₂O) to obtain 152 mg (yield 12%) of 3-{4-[4-(3-ethoxycarbonyl-4-oxo-6-fluoro-7-chloroquinoline-1-yl)butylamino]butyl}-6-(3-ethyl-4-methylanilino)uracil.

The above ester intermediate (105 mg, 0.16 mmol) was dissoved in methanol (6 ml), sodium hydroxide 2N (3 ml) was added to the solution and the mixture was stirred at room temperature overnight. The solvent was removed and the residue was purified by preparative HPLC (35% to 70% acetonitrile:H₂O) to obtain 115 mg (yield 91%) of compound 74 as trifluoroacetate. 400 MHz ¹H NMR (DMSO-d₆): 10.90 (s, 1H, NH), 9.03 (s, 1H, NH), 8.77 (s, 1H, FQ-C₂—H), 8.41 (d, 1H, FQ-C₅—H), 8.36 (s, 1H, NH), 8.16 (d, 1H, FQ-C8-H), 7.07 (d, 1H, ArH), 6.84-6.90 (m, 2H, ArH), 4.66 (s, 1H, C5-H), 4.55 (t, 2H, NCH₂), 3.64 (m, 2H, NCH₂), 2.83 (t, 4H, 2×NCH₂), 2.50 (q, 2H, ArCH₂CH₃), 2.16 (s, 3H, ArCH₃), 1.76 (m, 2H, CH₂), 1.55 (m, 2H, CH₂), 1.47 (m, 4H, 2×CH₂), 1.06 (t, 3H, ArCH₂CH₃).

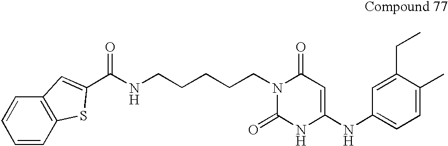

3-[5-(2-benzo[b]thiophenecarboxamido)pentyl]-6-(3-ethyl-4-methylanilino)uracil

3-(5-aminopentyl)-6-anilinouracil hydrochloride (77 mg, 0.21 mmol) was dissolved in dry pyridine (1 ml), benzo[b]thiophene-2-carbonyl chloride (50 mg, 0.25 mmol) and catalytic amount (10 mol %) of DMAP (4 mg, 0.03 mmol) were added and the mixture was stirred at room temperature overnight. The solvent was evaporated in vacuum, the residue dissolved in dichloromethane:methanol (10:1) and washed with aqueous ammonium choride. Organic fraction was dried over sodium sulfate, evaporated and purified by preparative TLC using dichloromethane:methanol (90:10) as eluent to give 12 mg (yield 7%) of compound 77 as a off-white solid. 400 MHz ¹H NMR (CD₃OD): 8.6 (s, 1H, NH), 7.82-7.91 (m, 5H, ArH), 7.12 (d, 1H, ArH) 6.97 (d, 1H, ArH), 6.89 (dd, 1H, ArH), 4.90 (s, 1H, C5-H), 3.85 (t, 2H, NCH₂), 3.38 (t, 2H, NCH₂), 2.60 (q, 2H, ArCH₂CH₃), 2.27 (s, 3H, ArCH₃), 1.64-1.69 (m, 4H, 2×CH₂), 1.39-1.43 (m, 2H, CH₂), 1.17 (t, 3H,ArCH₂CH₃).

General Method for the Preparation of 3-(sulfonylaminoalkyl)-6-anilinouracils

Method IV:

The organic sulfonyl chloride (1.1 eq) was added to a stirred solution of 3-(aminoalkyl)-6-anilinouracil hydrochloride (1 eq) and triethylamine (2.5-3.0 eq) in anhydrous methylene chloride (or chloroform or ethanol) at room temperature. The reaction mixture was stirred at room temperature for 3-5 hours. Once the reaction was complete, as monitored by thin layer chromatography, the mixture was concentrated in vacuo. Water was added, and the mixture was extracted with chloroform. After drying the extracts over sodium sulfate, the solvent was removed and the residue was purified by chromatography on silica gel using chloroform:methanol as eluent to give the product.

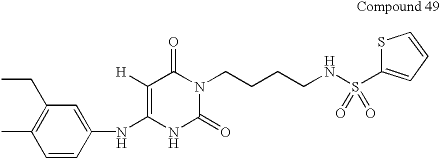

3-[4-(2-thienylsulfonylamino)butyl]-6-(3-ethyl-4-methylanilino)uracil

Method IV gave the product in 88% yield. 300 MHz ¹H NMR (DMSO-d₆): δ 1.14 (t, 3H, CH₃CH₂Ar), 1.30-1.50 (m, 4H, 2×CH₂), 2.24 (s, 3H, CH₃Ar), 2.57 (q, 2H, CH₂Ar), 2.78 (m, 2H, CH₂NH), 3.60 (t, 2H, CH₂N), 4.73 (s, 1H, C₅—H), 6.92-7.15 (m, 3H, Ar—H), 7.16 (m, 1H), 7.53 (d, 1H), 7.78 (s, 1H, NH), 7.90 (d, 1H), 8.10 (s, 1H, NH), 10.40 (s, 1H, NH) ppm.

General Method for the Preparation of 3-[ω-(alkylcarbonylamino)alkyl]-6-anilinouracils

Method V:

The organic acid chloride (1.2 eq) was added to a stirred solution of 3-(ω-aminoalkyl)-6-anilinouracil hydrochloride (1 eq) and triethylamine (2.5-3.0 eq) in anhydrous methylene chloride (or chloroform or ethanol) at room temperature. The reaction mixture was stirred at room temperature for 3-5 hours. Once the reaction was complete, as monitored by thin layer chromatography, the mixture was concentrated in vacuo. Water was added and extracted with chloroform, and the extracts were dried over Na₂SO₄. After removal of chloroform, the residue was purified by chromatography on silica gel using chloroform:methanol eluent to give the product.

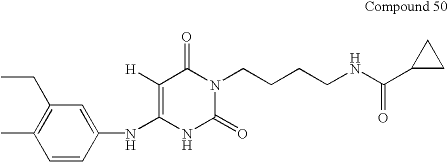

3-{4-[(cyclopropylcarbonyl)amino]butyl}-6-(3-ethyl-4-methylanilino)uracil

Method V gave the product in 86% yield. 300 MHz ¹H NMR (DMSO-d₆): δ 0.6 (m, 4H, 2×CH₂), 1.14 (t, 3H, CH₃CH₂Ar), 1.30-1.53 (m, 6H, 3×CH₂), 2.24 (s, 3H, CH₃Ar), 2.57 (q, 2H, CH₂Ar), 3.0 (m, 2H, CH₂NH), 3.65 (t, 2H, CH₂N), 4.73 (s, 1H, C₅—H), 6.92-7.15 (m, 3H, Ar—H), 8.0 (s, 1H, NH), 8.10 (s, 1H, NH), 10.40 (s, 1H, NH) ppm.

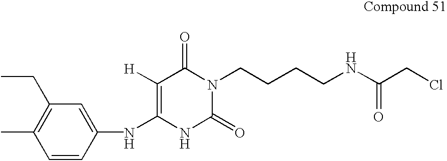

3-{4-[(chloromethylcarbonyl)amino]butyl}-6-(3-ethyl-4-methylanilino)uracil

Method V gave the product in 51% yield. 300 MHz ¹H NMR (DMSO-d₆): δ 1.14 (t, 3H, CH₃CH₂Ar), 1.30-1.60 (m, 4H, 2×CH₂), 2.24 (s, 3H, CH₃Ar), 2.57 (q, 2H, CH₂Ar), 3.07 (m, 2H, CH₂NH), 3.68 (t, 2H, CH₂N), 4.05 (s, 2H, CH₂Cl), 4.73 (s, 1H, C₅—H), 6.92-7.15 (m, 3H, Ar—H), 8.10 (s, 1H, NH), 8.22 (s, 1H, NH), 10.40 (s, 1H, NH) ppm.

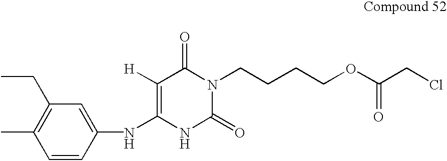

3-[4-(2-chloroacetoxy)butyl]-6-(3-ethyl-4-methylanilino)uracil

Chloroacetyl chloride (1.1 eq) was added to a stirred solution of 3-(4-hydroxybutyl)-6-anilinouracil (1 eq) and triethylamine (1.2 eq) in anhydrous N,N-dimethylformamide at room temperature. The reaction mixture was stirred at room temperature for 5 hours. The mixture was concentrated in vacuo, water was added, and the mixture extracted with chloroform and dried over sodium sulfate. After removal of chloroform, the residue was purified by chromatography on silica gel using chloroform:methanol eluent to give 3-[4-(2-chloroacetoxy)butyl]-6-(3-ethyl-4-methylanilino)uracil (yield 41%) as a white solid. 300 MHz ¹H NMR (DMSO-d₆): δ 1.14 (t, 3H, CH₃CH₂Ar), 1.57 (m, 4H, 2×CH₂), 2.24 (s, 3H, CH₃Ar), 2.57 (q, 2H, CH₂Ar), 3.69 (t, 2H, CH₂N), 4.16 (t, 2H, CH₂O), 4.40 (s, 2H, CH₂Cl), 4.73 (s, 1H, C₅—H), 6.92-7.15 (m, 3H, Ar—H), 8.18 (s, 1H, NH), 10.48 (s, 1H, NH) ppm.

General Method for the Synthesis of 3-substituted-6-(3-ethyl-4-methylanilino) Isocytosines

Method VI:

A mixture of 6-anilinoisocytosine (1 eq.), potassium carbonate (2 eq), alkylating agent (1.2 eq.) and a catalytic amount of benzyltriethylammonium chloride in acetone was heated at 60° C. for 36 hours. The solvent was removed in vacuo, and the residue was purified on a silica gel column with chloroform:methanol as eluent to afford the title product and the O4 isomeric product.

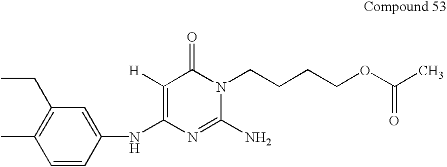

3-(4-Acetoxybutyl)-6-(3-ethyl-4-methylanilino)isocytosine

Method VI using 4-acetoxybutyl bromide as alkylating agent gave 8% yield of the title product, together with 72% yield of 04 isomeric product. Compound 52: 300 MHz ¹H NMR (DMSO-d₆): δ 1.05 (t, 3H, CH₃CH₂Ar), 1.55 (m, 4H, 2×CH₂), 2.02 (s, 3H, COCH₃), 2.20 (s, 3H, CH₃Ar), 2.50 (q, 2H, CH₂Ar), 3.80 (t, 2H, CH₂N), 4.00 (t, 2H, CH₂O), 4.90 (s, 1H, C₅—H), 6.85 (s, 2H, NH₂), 7.00-7.22 (m, 3H, Ar—H), 8.32 (s, 1H, NH) ppm.

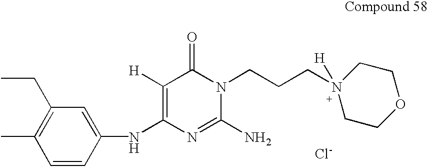

3-(3-Morpholinopropyl)-6-(3-ethyl-4-methylanilino)isocytosine Hydrochloride

Method VI using 3-morpholinopropyl bromide as alkylating agent gave an inseparable mixture of the 3-substituted (22%) and 04-substituted isomers (62%). A solution of this mixture in methanol was treated with an excess of a solution of hydrogen chloride in dioxane. The solvents were removed in vacuo, and the residue was purified by HPLC on a Vydac C18 column (15 micron). Elution with 25% acetonitrile in water containing 0.2% acetic acid and 0.1% triethylamine gave the product. 300 MHz ¹H NMR (DMSO-d₆): δ 1.18 (t, 3H, CH₃CH₂Ar), 1.80 (q, 2H, CH₂), 2.20 (s, 3H, CH₃Ar), 2.25-2.40 (m, 6H, 3×CH₂), 2.50 (q, 2H, CH₂Ar), 3.55 (m, 4H, 2×CH₂), 3.80 (t, 2H, CH₂N), 4.90 (s, 1H, C₅—H), 7.12 (s, 3H, NH₂ and NH⁺), 7.02-7.25 (m, 3H, Ar—H), 8.36 (s, 1H, NH) ppm.

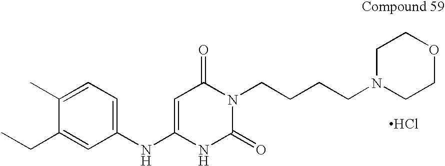

3-[4-(N-morpholino)butyl]-6-(3-ethyl-4-methylanilino)uracil Hydrochloride

A solution of 3-[4-(N-morpholino)butyl]-6-(3-ethyl-4-methylanilino)uracil (772 mg, 2 mmol) in CHCl₃:MeOH, 95:5 (20 ml) was treated with 1 ml of 4.0M HCl in dioxane. The mixture was stirred at room temperature for 1 hour. After removal of the solvent, the residue was dried in vacuo to provide the product in 98% yield. 300 MHz ¹H NMR (DMSO-d₆): 10.75 (s, 1H, HCl), 10.58 (s, 1H, NH), 8.75 (S, 1H, NH), 6.93-7.15 (m, 3H, Ar—H), 4.72 (s, 1H, C5-H), 3.90 (m, 2H, NCH₂), 3.80 (m, 4H, CH₂OCH₂), 3.38 (m, 2H, CH₂N), 3.12 (m, 4H, NCH₂×2), 2.60 (q, 2H, ArCH₂), 2.26 (s, 3H, ArCH₃), 1.48-1.55 (m, 4H, CH₂CH₂), 1.16 (t, 3H, ArCH₂CH₃) ppm.

The examples provided above are meant to illustrate the synthesis and characterization of a representative subset of the compounds of the invention. Analogous methods known to one skilled in the art can be used for the synthesis and characterization of other compounds of the invention (see, Advanced Organic Chemistry, J. March, 3rd. ed., NY: John Wiley (1985); The Chemistry of Functional Groups, S. Patai, Ed., NY: John Wiley, multiple volumes (1960ff); Heterocyclic and nucleoside synthesis—Purines, J. H. Lister, NY: Wiley-Interscience, (1971); Chemistry of Nucleosides and Nucleotides, Vols. 1 and 2, L. B. Townsend, Ed., NY: Plenum Press, (1988); Medicinal chemistry—The Basis of Medicinal Chemistry, 4th ed., 3 vols., M. E. Wolff, Ed., NY: Wiley-Interscience (1980), all incorporated herein by reference).

General Method for Preparation of 3-(4-Aryl-1-piperazinylbutyl)-6-anilinouracils

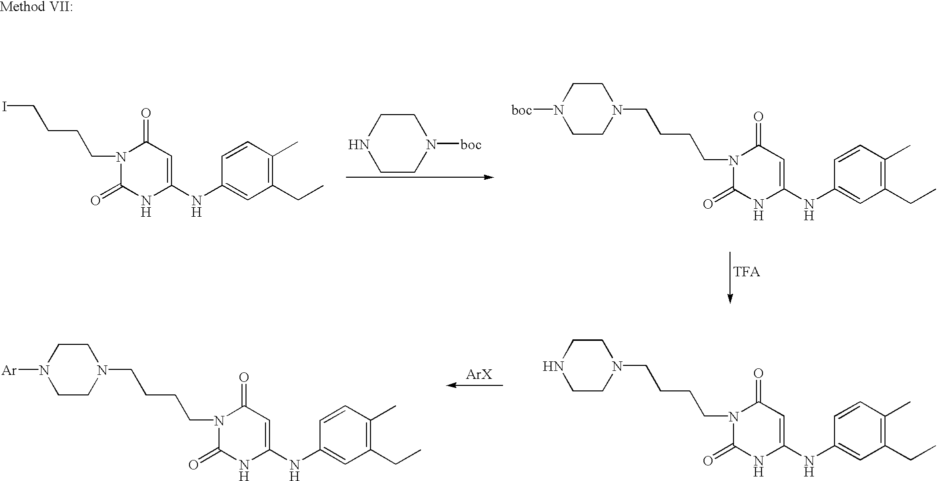

Method IIIb gave 3-(4-Boc-piperazinyl-butyl)-6-(3-ethyl-4-methylanilino)uracil intermediate in 60% yield after column purification.

The above Boc-piperazine intermediate (563 mg, 1.16 mmol) was dissolved in dichloromethane (20 ml) and trifluoroacetic acid (10 ml) was added to this solution. The mixture was stirred for 5 hours at room temperature, then the solvent was evaporated under reduced pressure and the residue was dried in vacuum to afford 675 mg (quantitative yield) of 3-(4-piperazinyl-butyl)-6-anilinouracil trifluoroacetate intermediate.

To a solution of the 3-(4-piperazinyl-butyl)-6-anilinouracil trifluoroacetate intermediate in dry acetonitrile was added corresponding chloropyrimidine (1.2 eq) in dry acetonitrile, followed by potassium carbonate (2 eq). Then, the mixture was refluxed under nitrogen for 5-6 hours, diluted with methanol (20 ml), filtered, solvent was evaporated to dryness and the residue was purified by column chromatography on silica gel using dichloromethane:methanol as eluent affording 3-(4-aryl-1-piperazinylbutyl)-6-anilinouracils.

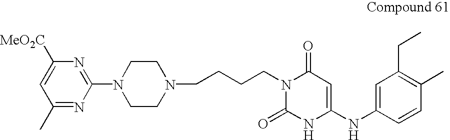

3-{4-[4-(6-methyl-4-methoxycarbonyl-2-pyrimidinyl)-1-piperazinyl]butyl}-6-(3-ethyl-4-methylanilino)uracil

Method VII gave the compound 61 in 58% yield after column purification. 400 MHz ¹H NMR (CDCl₃): 7.12 (d, 1H, ArH), 7.00 (s, 1H, Ar_{pyr}H), 6.90-6.99 (m, 2H, ArH), 5.09 (s, 1H, C5-H), 3.93 (s, 3H, CO₂CH₃), 3.84-3.91 (m, 6H, NCH₂ and 2×NCH_{2 pip}), 2.58 (q, 2H, ArCH₂CH₃), 2.35-2.46 (m, 6H, NCH₂ and 2×NCH_{2 pip}), 2.39 (s, 3H, Ar_{pyr}CH₃), 2.28 (s, 3H, ArCH₃), 1.66 (m, 2H, CH₂), 1.55 (m, 2H, CH₂), 1.18 (t, 3H, ArCH₂CH₃) ppm.

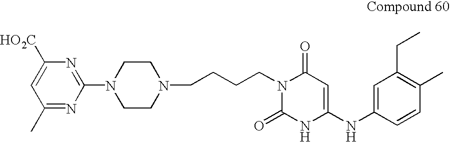

3-{4-[4-(6-methyl-4-carboxy-2-pyrimidinyl)-1-piperazinyl]butyl}-6-(3-ethyl-4-methylanilino)uracil

Compound 61 (35 mg, 0.08 mmol) was dissolved in a mixture of THF:H₂O (3:2) and lithium hydroxide was added. The mixture was stirred at room temperature for 6 hours, acidified by addition of 10% HCl to pH 2-3 and filtered through celite, washing with H₂O and THF—CH₂Cl₂-MeOH. Filtrate was evaporated to dryness and off-white solid dried in vacuum and triturated with ether and acetonitrile to afford 47 mg (yield 100%) of compound 60 as hydrochloride salt. 400 MHz ¹H NMR (DMSO-d₆): 11.57 (s, 1H, CO₂H), 10.94 (s, 1H, NH), 9.44 (s, 1H, NH), 7.10-7.13 (m, 2H, ArH and Ar_{pyr}H), 6.88-6.93 (m, 2H, ArH), 4.70-4.75 (m, 3H, C5-H and NCH₂), 3.68 (t, 2H, NCH₂), 3.48 (m, 4H, 2×NCH_{2 pip}), 2.95-3.13 (m, 4H, 2×NCH_{2 pip}), 2.55 (q, 2H, ArCH₂CH₃), 2.38 (s, 3H, Ar_{pyr}CH₃), 2.21 (s, 3H, ArCH₃), 1.71 (m, 2H, CH₂), 1.49 (m, 2H, CH₂), 1.09 (t, 3H, ArCH₂CH₃) ppm.

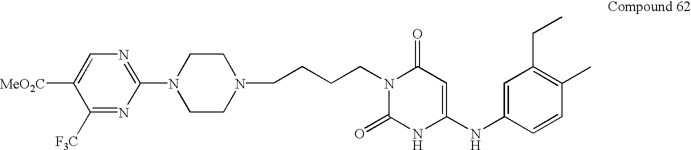

3-{4-[4-(4-trifluoromethyl-5-methoxycarbonyl-2-pyrimidinyl)-1-piperazinyl]butyl}-6-(3-ethyl-4-methylanilino)uracil

Method VII gave the compound 62 in 66% yield after column purification. 400 MHz ¹H NMR (CDCl₃): 8.81 (s, 1H, Ar_{pyr}H), 7.39 (s, 1H, NH), 7.01 (d, 1H, ArH), 6.81-6.88 (m, 2H, ArH), 5.02 (s, 1H, C₅—H), 3.89 (m, 4H, 2×NCH_{2 pip}), 3.81 (m, 5H, CO₂CH₃ and NCH₂), 2.47-2.52 (m, 6H, 2×NCH_{2 pip} and ArCH₂CH₃), 2.39 (m, 2H, NCH₂), 2.19 (s, 3H, ArCH₃), 1.59 (m, 2H, CH₂), 1.51 (m, 2H, CH₂), 1.09 (t, 3H, ArCH₂CH₃) ppm.

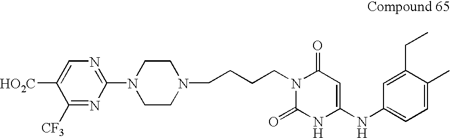

3-{4-[4-(4-trifluoromethyl-5-carboxy-2-pyrimidinyl)-1-piperazinyl]butyl}-6-(3-ethyl-4-methylanilino)uracil

Compound 62 (61 mg, 0.1 mmol) was dissolved in a mixture of THF:H₂O (1:1) and an excess of lithium hydroxide (10 eq) was added. The mixture was stirred at room temperature for 2 days, neutralized by addition of diluted acetic acid, evaporated to dryness and purified by column chromatography on silica gel using dichloromethane:methanol (95:5) as eluent to afford 27 mg (47% yield) of compound 65. 400 MHz ¹H NMR (DMSO-d6): 10.44 (s, 1H, NH), 8.9 (s, 1H, Ar_{pyr}H), 8.14 (s, 1H, NH), 7.12 (m, 1H, ArH), 6.90-6.95 (m, 2H, ArH), 4.71 (s, 1H, C5-H), 3.86 (m, 4H, 2×NCH₂), 3.68 (m, 2H, NCH₂), 2.18-2.65 (m, 6H, 3×NCH₂ and ArCH₂CH₃), 2.17 (s, 3H, ArCH₃), 1.49 (m, 4H, 2×CH₂),1.28 (t, 3H, ArCH₂CH₃) ppm.

General Method for the Synthesis of 3-[4-(aryl-1,2,3,6-tetrahydropyridinyl)]butyl-6-(3-ethyl-4-methylanilino)uracils

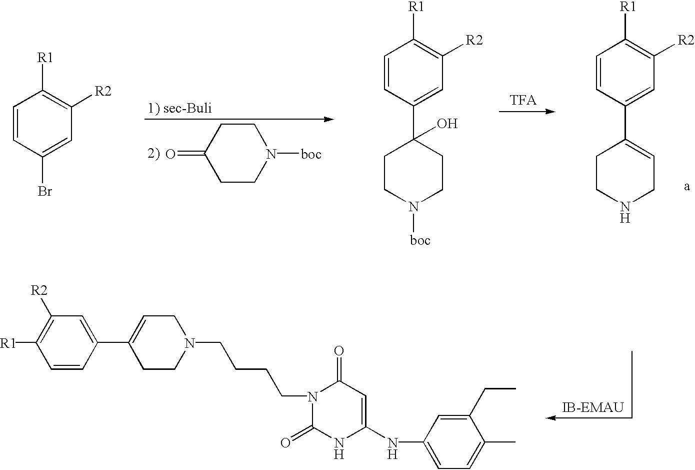

General Preparation of 4-aryl-1,2,3,6-tetrahydro-pyridines a:

Method VIII:

Aryl bromide (1 eq) was dissolved in dry cyclohexane (or THF or mixture cyclohexane/THF), cooled to −73° C. and sec-butyllithium (1.3M in cyclohexane; 1.05 eq) was added dropwise. The mixture was stirred for 1 hour at −40° C./−30° C., then it was cooled to −73° C. and a solution of Boc-piperidone (1 eq) in dry THF was added dropwise. The mixture was stirred for an additional 3 hours allowing to warm up to room temperature, then it was diluted with dichloromethane and quenched with H₂O. The mixture was washed with diluted aqueous HCl (1%) adjusting pH to 7, and aqueous fraction was extracted with dichloromethane. Combined organic fractions were dried over sodium sulfate, concentrated in vacuum and separated by column chromatography on silica gel using hexane:ethyl acetate as eluent to give Boc-piperidine intermediate.

Boc-piperidine intermediate was dissolved in dichloromethane, an excess of trifluoroacetic acid (1% v/v) was added dropwise to this solution and the mixture was stirred under nitrogen at room temperature for 24 hours. Then the mixture was evaporated to dryness. The residue was triturated with dry ether and dried in vacuum affording 4-aryl-1,2,3,6-tetrahydro-pyridine a.

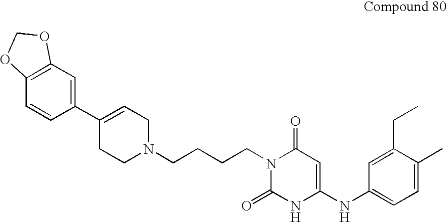

3-{4-[4-(3,4-dioxomethylenephenyl-1,2,3,6-tetrahydropyridinyl)]butyl}-6-(3-ethyl-4-methylanilino)uracil

(3,4-dioxomethylenephenyl)-1,2,3,6-tetrahydropyridine was obtained in 35% yield according to the method VIII.

Method IIIb afforded the compound 80 in 74% yield. 400 MHz ¹H NMR (CD₃OD): 7.14 (d, 1H, Ar—H), 7.02 (d, 1H, ArH), 6.92-6.96 (m, 3H, ArH), 6.74 (d, 1H, ArH), 5.98 (s, 1H, CH), 5.91 (s, 2H, OCH₂O), 4.87 (s, 1H, C5-H), 3.89 (t, 2H, NCH₂), 3.15 (m, 2H, NCH₂), 2.74 (t, 2H, NCH₂), 2.65 (q, 2H, ArCH₂CH₃), 2.52 (m, 4H, CH₂ and NCH₂), 2.28 (s, 3H, ArCH₃), 1.61-1.63 (m, 4H, 2×CH₂), 1.20 (t, 3H, ArCH₂CH₃).

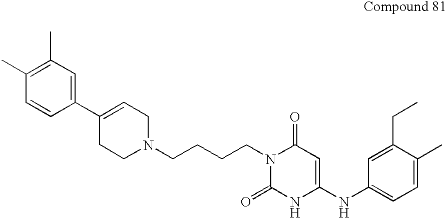

3-{4-[4-(3,4-(dimethyl)phenyl)1,2,3,6-tetrahydropyridinyl)]butyl}-6-(3-ethyl-4-methylanilino)uracil

(3,4-dimethylphenyl)1,2,3,6-tetrahydropyridine was obtained in 57% yield according to the method VIII.

Method IIIb gave the compound 81 in 73% yield. 400 MHz ¹H NMR (DMSO-d₆): 7.14 (s, 1H, Ar—H), 7.0-7.09 (m, 5H, ArH), 6.0 (m, 1H, CH), 4.75 (s, 1H, C5-H), 3.70 (t, 2H, NCH₂), 2.96 (m, 2H, NCH₂), 2.46-2.54 (m, 4H, 2NCH₂), 2.31-2.36 (m, 4H, CH₂ and ArCH₂CH₃), 2.15-2.17 (3s, 9H, 3ArCH₃), 1.40-1.48 (m, 4H, 2CH₂), 1.11 (t, 3H, ArCH₂CH₃).

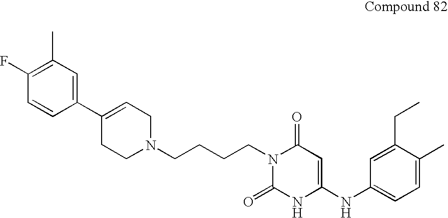

3-{4-[4-(4-fluoro-3-methyl)phenyl-1,2,3,6-tetrahydropyridinyl)]butyl}-6-(3-ethyl-4-methylanilino)uracil

(4-fluoro-3-methyl)phenyl-1,2,3,6-tetrahydropyridine was obtained in 39% yield according to the method VIII.

Method IIIb gave the compound 82 in 65% yield. 400 MHz ¹H NMR (DMSO-d₆): 8.17 (s, 1H, NH), 7.31 (d, 1H, ArH), 7.23 (m, 1H, ArH), 7.12 (d, 1H, ArH), 7.05 (dd, 1H, ArH), 6.93 (m, 2H, ArH), 6.06 (m, 1H, CH), 4.72 (s, 1H, C5-H), 3.69 (t, 2H, NCH₂), 3.01 (m, 2H, NCH₂), 2.52-2.58 (m, 4H, CH₂ and ArCH₂CH₃), 2.35-2.40 (m, 4H, 2CH₂), 2.21 (s, 3H, Ar—CH₃), 1.43-1.5 (m, 4H, 2CH₂), 1.12 (t, 3H, ArCH₂CH₃).

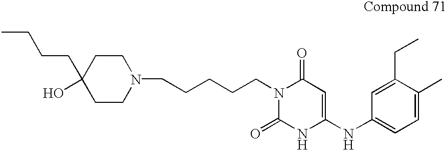

3-{5-[1-(4-hydroxy-4-butylpiperidinyl)]pentyl}-6-(3-ethyl-4-methylanilino)uracil

1-Benzyl-4-hydroxy-4-butylpiperidine (0.53 g, 2.15 mmol) was dissolved in ethanol (50 ml), palladium (10% on activated charbon, 226 mg) was added and the mixture was deoxygenated by bubbling nitrogen through solution for 15 minutes. Then the flask was filled with hydrogen and the mixture was stirred under hydrogen at room temperature for 2 days. The mixture was filtered through a celite pad washing with ethanol, then with methanol and solvent was evaporated in vacuum affording 312 mg (yield 100%) of unprotected piperidine intermediate as a yellow oil.

The above piperidine intermediate (52 mg, 0.33 mmol) and IP-EMAU (73 mg, 0.16 mmol) were dissolved in dry DMF (3 ml), sodium carbonate (35 mg, 0.33 mmol) was added and the mixture was stirred at room temperature for 2 days. Then, DMF was evaporated to dryness, the residue dissolved in the mixture of dichloromethane and methanol, washed with H₂O, dried over sodium sulfate and solvent was evaporated under reduced pressure. The residue was purified by column chromatography using dichloromethane:methanol (gradient from 100:0 to 95:5) as eluent affording 51 mg (68%) of compound 71 as as a yellow solid. 400 MHz ¹H NMR (CDCl₃): 10.32 (s, 1H, NH), 7.16 (d, 1H, ArH), 6.90 (dd, 1H, ArH), 6.86 (dd, 1H, ArH), 3.81 (t, 2H, NCH₂), 3.5 (s, 1H, C5-H), 3.15-3.33 (m, 6H, 3×NCH₂), 3.06 (q, 2H, CH₂), 2.88 (m, 2H, CH₂), 2.56 (q, 2H, ArCH₂CH₃), 2.20-2.28 (m, 7H, ArCH₃ and 2×CH₂), 1.59-1.89 (m, 8H, 4×CH₂), 1.48 (t, 3H, CH₃), 1.14 (t, 3H, ArCH₂CH₃).

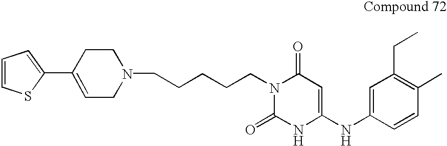

3-{5-[1-(4-(thiophen-2-yl)-1,2,3,6-tetrahydropyridinyl)]pentyl}-6-(3-ethyl-4-methylanilino)uracil

2-Bromothiophene (4.02 g, 24.65 mmol) was dissolved in dry THF (50 ml); this solution was cooled to −73° C. and n-butyllithium (2M in pentane; 12.94 ml, 25.89 mmol) was added dropwise. The mixture was brought to −30° C. and stirred at this temperature for 2 hours. Then it was cooled back to −73° C. and a solution of 1-benzyl-4-piperidone (4.67 g, 24.65 mmol) in dry THF (20 ml) was added dropwise. The mixture was allowed to warm up to room temperature (over 3 hours), stirred for an additional hour and quenched by addition of H₂O. Then it was diluted with dichloromethane and washed wih saturated aqueous ammonium chloride. Organic fraction was separated, dried over sodium sulfate and concentrated. The residue was taken into ether and insoluble white solid filtered off. Filtrate evaporated to dryness affording 4.82 g (yield 72%) of benzyl-piperidine intermediate which was used in the next step without further purification.

The above intermediate (2.15 g, 7.9 mmol) was dissolved in toluene (120 ml), p-toluenesulfonic acid (1.65 g, 8.7 mmol) was added and the mixture was refluxed for 5 hours separating H₂O. Then the mixture was brought to room temperature and filtered through a celite pad. Obtained solution was neutralized with aqueous NaOH (1N) and washed with water. The solution was dried over sodium sulfate and solvent evaporated under reduced pressure to afford the dehydro-intermediate in a quantitative yield.

To a solution of the above intermediate (0.52 g, 2.05 mmol) in dichloromethane (20 ml) was added phenyl chloroformate (0.96 g, 6.14 mmol) and the mixture was stirred under nitrogen at room temperature overnight. Then, the mixture was concentrated under reduced pressure and an excess of chloroformate and benzyl choride were distilled off in vacuum. The crude material was dissolved in THF (100 ml) containing 5% mol of 18-crown-6 (26 mg, 0.1 mmol), powdered KOH (345 mg, 6.14 mmol) was added to the mixture and the reaction mixture was refluxed under nitrogen for 2 days. Then the mixture was diluted with dichloromethane and washed with aqueous ammonium chloride. Aqueous fraction was separated and extracted with dichloromethane and dichloromethane-methanol. Combined organic fractions were dried over sodium sulfate, solvent evaporated to dryness and the residue was separated by column chromatography using dichloromethane-methanol (gradient from 100:0 to 80:20) as eluent affording 179 mg (yield 53%) of amine.

The above amine (81 mg, 0.49 mmol) and IP-EMAU (108 mg, 0.24 mmol) were dissolved in dry DMF (3 ml), sodium carbonate (52 mg, 0.49 mmol) was added to the mixture which was stirred at room temperature under nitrogen for 3 days. The solvent was evaporated to dryness, the residue was dissolved in dichloromethane:methanol (9:1) and washed with water. Aqueous fraction was separated and extracted with dichloromethane: methanol. Combined organic fractions were dried over sodium sulfate, concentrated in vacuum and separated by column chromatography on silica gel using dichloromethane:methanol (gradient from 100:0 to 90:10) as eluent affording 67 mg (yield 58%) of compound 72 as a yellow solid. 400 MHz ¹H NMR (CDCl₃): 8.21 (s, 1H, NH), 7.25 (d, 1H, ArH), 7.17 (dd, 1H, ArH), 6.88-7.03 (m, 6H, ArH and ArₜₕᵢₒH), 5.97 (s, 1H, CH), 4.98 (s, 1H, C5-H), 3.82 (m, 4H, 2×NCH₂), 3.41 (m, 2H, NCH₂), 3.13 (t, 2H, NCH₂), 2.89 (m, 2H, CH₂), 2.52 (q, 2H, ArCH₂CH₃), 2.23 (s, 3H, ArCH₃), 1.92 (m, 2H, CH₂), 1.65 (m, 2H, CH₂), 1.39 (m, 2H, CH₂), 1.12 (t, 3H, ArCH₂CH₃).

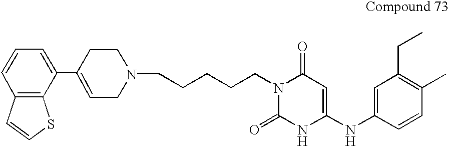

3-{5-[1-(4-(benzo[b]thiophen-7-yl)-1,2,3,6-tetrahydropyridinyl)]pentyl}-6-(3-ethyl-4-methylanilino)uracil

Thiophenol (3.2 g, 29.2 mmol) in dry cyclohexane (65 ml) was slowly added to a solution of TMEDA (7.47 g, 64.3 mmol) and n-butyllithium (2M in cyclohexane; 32.1 ml, 64.3 mmol) in dry cyclohexane (65 ml) at room temperature under nitrogen. The reaction mixture was stirred at room temperature for 3 hours, then was heated at 50° C. for 5 hours and left at room temperature overnight. A solution of N-Boc-piperidone (5.5 g, 27.8 mmol) in cyclohexane (10 ml) was added to the lithio reagent at 0° C., the mixture was stirred for 30 min at 0° C. then for 24 h at room temperature. Then dimethyl acetal of 2-bromoacetaldehyde (5.18 g, 30.7 mmol) was added dropwise and the mixture was stirred at room temperature for another 24 hours. The mixture was quenched by addition of H₂O, diluted with dichloromethane and washed with aqueous ammonium chloride. The aqueous fraction was back-extracted with dichloromethane. Organic fractions were dried over sodium sulfate, concentrated and separated by column chromatography on silica gel using hexane-ethylacetate (gradient from 100:0 to 50:50) as eluent to obtain 3.32 g (yield 30%) of piperidine derivative.

Piperidine derivative (1.67 g, 4.21 mmol) was dissolved in toluene (100 ml). P-toluenesulfonic acid (1.6 g, 8.42 mmol) was added and the mixture was refluxed with Dean-Stark condensor for 4 hours. Then the mixture was brought to room temperature and filtered through a celite pad. Solvent was evaporated and the residue purified by column chromatography using dichloromethane:methanol (gradient from 100:0 to 80:20) as eluent affording 93 mg (yield 10%) of 4-(7-benzo[b]thiophenyl)-3,4-dehydropiperidine.

The above amine (71 mg, 0.33 mmol) and IP-EMAU (145 mg, 0.33 mmol) were dissolved in dry DMF (5 ml). Sodium carbonate (70 mg, 0.66 mmol) was added and the mixture was stirred overnight at room temperature. Then DMF was evaporated to dryness and the residue purified by column chromatography using dichloromethane:methanol (gradient 100:0 to 80:20) affording 132 mg (yield 76%) of compound 73. 400 MHz ¹H NMR (CDCl₃): 8.25 (s, 1H, NH), 7.99 (s, 1H, NH), 7.68 (d, 1H, ArH), 6.74-7.09 (m, 7H, ArH), 6.12 (s, 1H, CH), 4.94 (s, 1H, C5-H), 3.80-3.85 (m, 4H, 2×NCH₂), 3.42 (m, 2H, NCH₂), 3.22 (m, 2H, NCH₂), 2.44 (q, 2H, ArCH₂CH₃), 2.16 (s, 3H, ArCH₃), 1.94 (m, 2H, CH₂), 1.61-1.77 (m, 4H, 2×CH₂), 1.38 (m, 2H, CH₂), 1.05 (t, 3H, ArCH₂CH₃).

### Example 4

### Pharmacokinetic Properties of Compound 59

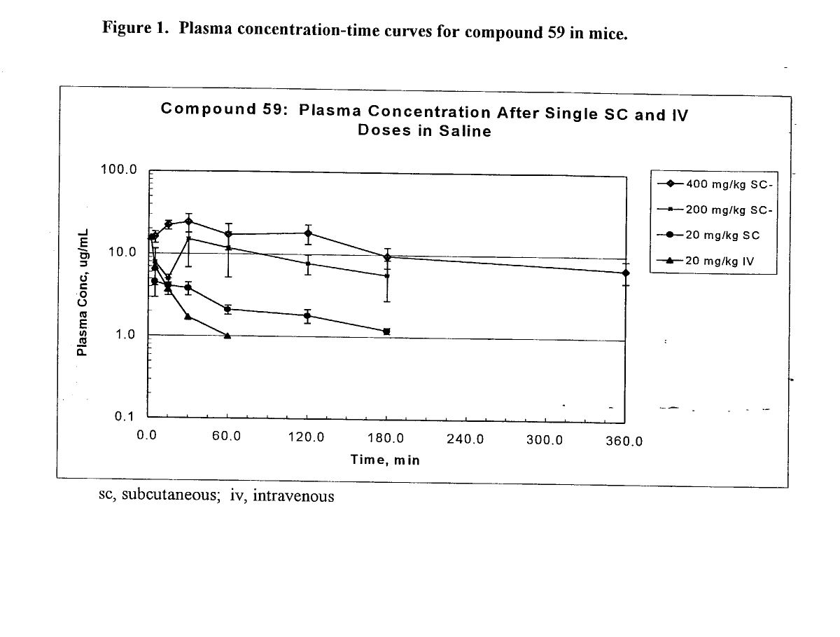

Compound 59 was given to mice in simple saline solutions by various routes—subcutaneous (sc), intravenous (iv), and orally (po). High performance liquid chromatography (HPLC) on a reverse phase C 18 column was used to monitor the concentration of the compound in animal plasma, and the results were plotted as a function of time, as shown in FIG. 1. Various pharmacokinetic parameters derived from this data are summarized in Table 2, below.

TABLE 2
Pharmacokinetic parameters of Compound 59 in mice

| PK | | Route of Administration (saline solution) | | | |
|---|---|---|---|---|---|
| Parameters | Units | iv | sc | sc | sc |
| Dose | mg/kg | 20 | 20 | 200 | 400 |
| Tmax | min | 0 | 5.00 | 30.0 | 30.0 |
| | | (ncm fit¹) | | | |
| Cmax | ug/mL | 27.4 | 4.60 | 15.0 | 24.1 |
| T1/2 | min | 39 | 149 | 132 | 373 |
| AUC(0-t) | ug-min/mL | 234 | 406 | 1613 | 4500 |
| | | 0-120 | (0-180) | (0-180) | (0-360) |
| AUC(0-60) | ug-min/mL | 204 | 200 | 635 | 1191 |
| Vd | L/kg | 4.3 | 6.5 | nc² | nc |
| Cl | L/h/kg | 4.62 | 1.81 | nc | nc |

¹ncm fit = noncompartmental fit by winnonlin program
²nc = not calculated

Tmax, time to peak concentration; Cmax, peak concentration; T½, half-life; AUC(0-t), area under the curve, from 0 to time t; AUC(0-60), area under the curve, from 0 to 60 minutes; Vd, volume of distribution; Cl, clearance.

These results show that Compound 59 was completely absorbed by the sc route, and that the apparent T½ (half life) of compound in plasma was prolonged relative to that by the iv route. The compound was about 50% absorbed from the po route, i.e., it had about 50% oral bioavailability.

### Example 5

### Efficacy of Compounds 12, 19, 26, and 29

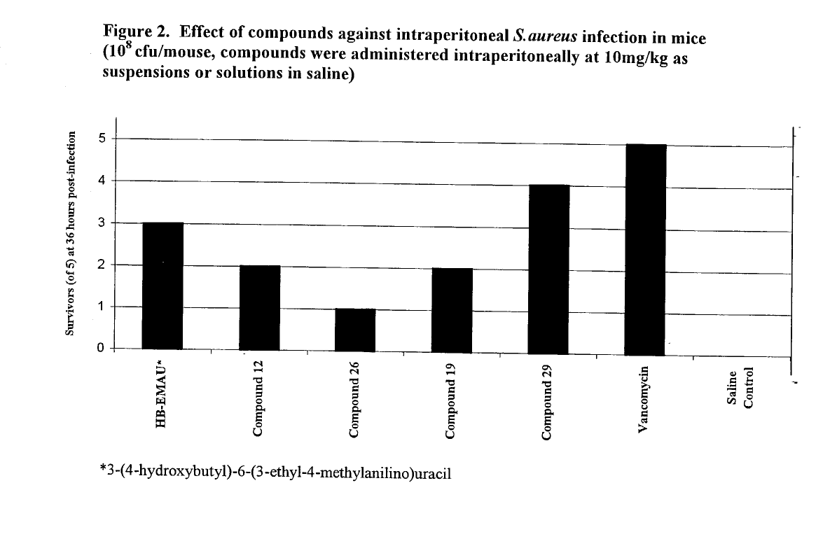

Compounds 12, 19, 26, and 29 were given by the intraperitoneal (ip) route as suspensions in saline to mice previously infected with S. aureus (Smith strain). Vancomycin was used as a positive drug control and saline was used as a negative vehicle control. As shown in FIG. 2, Compound 29 protected 4 of the 5 animals, whereas Compound 26 protected only 1 of the 5 animals. In this experiment, the positive control drug vancomycin protected all animals, and the negative vehicle control protected no animals.

### Example 6

### Efficacy of Compound 59

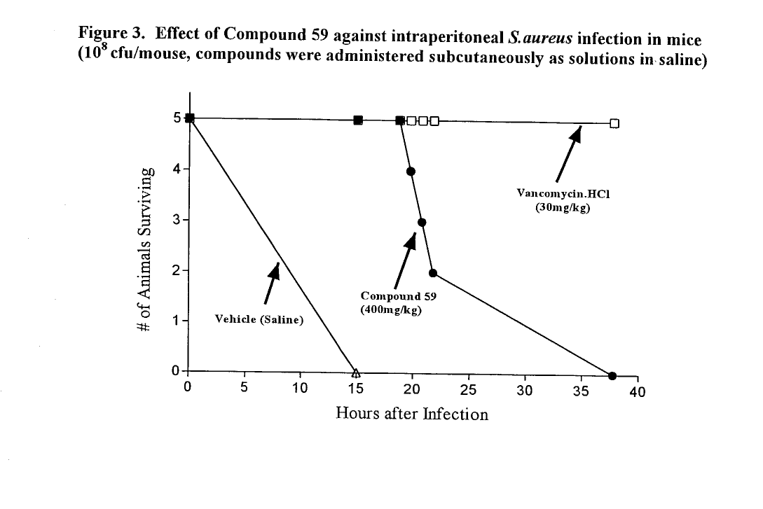

Compound 59 was give by the sc route to mice infected by the ip route with S. aureus (Smith strain). As shown in FIG. 3, Compound 59 prolonged the survival time of mice relative to vehicle control. Vancomycin also protected the animals from this infection.

### Other Embodiments

A number of embodiments of the invention have been described. Nevertheless, it will be understood that various modifications can be made without departing from the spirit and scope of the invention.

What is claimed:

1. A compound having the formula 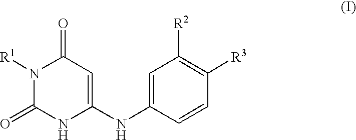
wherein R¹ is (CH₂)ₘ-{(A)ₙ-(CH₂)ₚ}_{q}—B,
in which each A is, independently, CH₂, CH═CH, C≡C, CO, O, S, NR⁸, where R⁸ is H or C₁₋₆ alkyl, CHR¹⁰, where R¹⁰ is OH or C₁₋₆ alkyl, CH(CR¹²R¹³)ᵣCH, where each of R¹² and R¹³ is, independently, H, halogen, or C₁₋₆ alkyl, OCO, CONR¹⁴, NR¹⁵CO, where each of R¹⁴ and R¹⁵ is, independently, H or C₁₋₆ alkyl, SO₂NH, or NHSO₂;
B is H, halogen, substituted or unsubstituted C₁₋₁₀ alkyl, C₃₋₈ cycloalkyl, C₅₋₁₅ heteroaryl, NH₂, CN, OR¹⁶, SR¹⁸, COR⁹, OCOR²⁰, NR²¹(CO)R²², NR²³R²⁴, NR²⁵(CO)NHR²⁶, CN, CH(CO₂R²⁸)₂, CO₂R³⁰, NHSO₂R³², CONR³⁴R³⁶, or CH₂COR³⁸, in which each of R¹⁶-R³⁸ is, independently, H, substituted or unsubstituted C₁₋₆ alkyl, substituted or unsubstituted C₁₋₆ cycloalkyl, substituted or unsubstituted C₆₋₁₂ aryl, substituted or unsubstituted C₇₋₂₀ arylalkyl, substituted or unsubstituted C₇₋₂₀ alkylaryl, substituted or unsubstituted C₄₋₁₀ heteroaryl, C₁₋₃ acyl, or C₁₋₆ sulfonyl, or B is a substituted or unsubstituted 5-8 membered non-aromatic heterocycle;
wherein m is 1-4, n is 0 or 1, p is 0-4, q is 0-4, and r is 1-4; and
wherein each of R² and R³ is, independently, C₁₋₆ alkyl, C₂₋₆ alkenyl, or halogen, or R² and R³ together are C₃₋₅ alkylene,
provided that R¹ is not unsubstituted alkyl, hydroxy-substituted alkyl, alkoxy-substituted alkyl, carboxy-substituted alkyl, amino-substituted alkyl, (substituted-amino)-substituted alkyl, amido-substituted alkyl, carbamate-substituted alkyl, halogen-substituted alkyl, thio-substituted alkyl, azido-substituted alkyl, dithio-substituted alkyl, sulfonyl-substituted alkyl, or alkenyl;
further provided that when A is OCO, then B is not CO₂H, NH₂, or CH(CO₂R)₂;
further provided that when A is C═C, then B is not H or alkyl; and
further provided that when A is NH, then B is not CO₂R;
or a pharmaceutically acceptable salt thereof.

2. The compound of claim 1, wherein n is 1, p is 1-4, q is 1, A is not CH₂, C═C, or C≡C, and B is not H, alkyl, or cycloalkyl.

3. The compound of claim 1, wherein n is 1, q is 1, and A is not CH₂, and wherein B is a 5-8 membered, substituted, or unsubstituted non-aromatic heterocycle.

4. The compound of claim 3, wherein B includes a substituent that is capable of forming a salt with an acid or a base.

5. The compound of claim 4, wherein B includes an amine, a carboxylic acid, a sulfonamide, or an imide.

6. The compound of claim 3, wherein B includes a ring N atom that is capable of forming a salt with an acid or a base.

7. The compound of claim 6, wherein B includes an amine, a sulfonamide, or an imide.

8. The compound of claim 1, wherein B is a substituted heterocycle.

9. The compound of claim 8, wherein B includes a substituent that is capable of forming a salt with an acid or a base.

10. The compound of claim 9, wherein B includes an amine, a carboxylic acid, a sulfonamide, or an imide.

11. The compound of claim 8, wherein B includes a ring N atom that is capable of forming a salt with an acid or a base.

12. The compound of claim 11, wherein B includes an amine, a sulfonamide, or an imide.

13. The compound of claim 1, wherein R² is selected from the group consisting of Cl, Br, I, CH₃, CH₂CH₃, and CH═CH₂.

14. The compound of claim 1, wherein R² is CH₂CH₃.

15. The compound of claim 14, wherein R³ is CH₃.

16. The compound of claim 1, wherein A is O.

17. The compound of claim 1, wherein B is 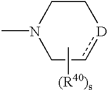
in which D is O, S, NR⁴¹, or C(R⁴²)(R⁴³), in which each of R⁴¹-R⁴³ is, independently, absent, H, C₁₋₆ alkyl, C₆₋₂₀ aryl, C₄₋₉ heteroaryl, C₇₋₁₂ arylalkyl, or COR⁴⁴, in which R⁴⁴ is substituted or unsubstituted C₆₋₁₀ aryl or substituted or unsubstituted C₄₋₆ heteroaryl, OR⁴⁵, in which R⁴⁵ is H, substituted or unsubstituted C₁₋₆ alkyl, substituted or unsubstituted C₁₋₆ cycloalkyl, C₆₋₁₂ aryl, C₇₋₂₀ arylalkyl, C₄₋₆ heteroaryl, C₁₋₃ acyl, or C₁₋₆ sulfonyl;
each R⁴⁰ is, independently, substituted or unsubstituted C₁₋₆ alkyl, substituted or unsubstituted C₁₋₆ cycloalkyl, C₆₋₁₂ aryl, C₇₋₂₀ arylalkyl, C₄₋₆ heteroaryl, C₁₋₃ acyl, or C₁₋₆ sulfonyl, OR⁴⁶, CH₂OR⁴⁸, in which each of R⁴⁶ and R⁴⁸ is, independently, H, substituted or unsubstituted C₁₋₆ alkyl, substituted or unsubstituted C₁₋₆ cycloalkyl, C₆₋₁₂ aryl, C₇₋₂₀ arylalkyl, C₄₋₆ heteroaryl, C₁₋₃ acyl, or C₁₋₆ sulfonyl, or CO₂R⁵⁰, where R⁵⁰ is substituted or unsubstituted C₆₋₁₂ aryl, or substituted or unsubstituted C₄₋₆ heteroaryl;
and s is 0-2.

18. The compound of claim 17, wherein D is O, S, or NR⁴′, and s is 1 or 2.

19. The compound of claim 17, wherein D is C(R⁴²)(R⁴³), and wherein at least one of R⁴² and R⁴³ is not H.

20. The compound of claim 1, wherein the compound is an acid salt derived from an inorganic acid.

21. The compound of claim 20, wherein the compound is selected from the group consisting of hydrochlorides, hydrobromides, and sulfates.

22. The compound of claim 1, wherein the compound is an acid salt derived from an organic acid.

23. The compound of claim 22, wherein the compound is selected from the group consisting of mesylates, maleates, and fumarates.

24. A pharmaceutical composition comprising a compound of claim 1 and a pharmaceutically acceptable carrier.

25. A method of inhibiting growth of Gram-positive bacteria in vitro, the method comprising contacting the bacteria with an effective amount of a compound of claim 1.

26. A method of inhibiting growth of Gram-positive bacteria, the method comprising contacting the bacteria with an effective amount of a compound of claim 1.

27. A method of treating a subject with a Gram-positive bacterial infection, the method comprising administering to the subject a therapeutically effective amount of a compound of claim 1.

28. A method of treating a subject with a mycoplasmal infection, the method comprising administering to the subject a therapeutically effective amount of a compound of claim 1.

29. A method of prophylactically treating a subject susceptible to a Gram-positive bacterial infection, the method comprising administering to the subject a therapeutically effective amount of a compound of claim 1.

30. A method of prophylactically treating a subject susceptible to a mycoplasmal infection, the method comprising administering to the animal a therapeutically effective amount of a compound of claim 1.

31. A compound having the formula 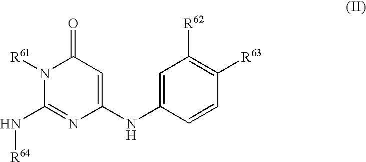
wherein R⁶¹ is (CH₂)ₜ-{(E)ₙ—(CH₂)ᵥ}_{w}-J,
in which each E is, independently, CH₂, CH═CH, C≡C, CO, O, S, NR⁷⁰, where R⁷⁰ is H or C₁₋₆ alkyl, CHR⁷¹, where R⁷¹ is OH or C₁₋₆ alkyl, CH(CR⁷²R⁷³)CH, where each of R⁷² and R⁷³ is, independently, H, halogen, or C₁₋₆ alkyl, OCO, CONR⁷⁴, NR⁷⁵CO, where each of R⁷⁴ and R⁷⁵ is, independently, H or C₁₋₆ alkyl, SO₂NH, or NHSO₂;
J is H, halogen, substituted or unsubstituted C₁₋₁₀ alkyl, C₃₋₈ cycloalkyl, C₅₋₁₀ heteroaryl, NH₂, CN, OR₇₆, SR⁷⁸, COR⁷⁹, OCOR⁸⁰, NR⁸¹(CO)R⁸², NR⁸³R⁸⁴, NR⁸⁵(CO)NHR⁸⁶, CN, CH(CO₂R⁸⁸)₂, CO₂R⁹⁰, NHSO₂R⁹², CONR⁹⁴R⁹⁶, or CH₂COR⁹⁸, in which each of R⁷⁶-R⁹⁸ is, independently, H, substituted or unsubstituted C₁₋₆ alkyl, substituted or unsubstituted C₁₋₆ cycloalkyl, substituted or unsubstituted C₆₋₁₂ aryl, substituted or unsubstituted C₇₋₂₀ arylalkyl, substituted or unsubstituted C₇₋₂₀ alkylaryl, substituted or unsubstituted C₄₋₁₀ heteroaryl, C₁₋₃ acyl, or Ci-6 sulfonyl, or B is a substituted or unsubstituted 5-8 membered non-aromatic heterocycle;
wherein R⁶⁴ is H, substituted or unsubstituted C₁₋₆ alkyl, or substituted or unsubstituted C₆₋₁₂ aryl;
wherein each of R⁶² and R⁶³ is, independently, C₁₋₆ alkyl, C₂₋₆ alkenyl, or halogen, or R⁶² and R⁶³ together are C₃₋₅ alkylene; and
wherein t is 1-4, u is 0 or 1, v is 0-4, w is 0-4, and x is 1-4;
provided that R⁶¹ is not unsubstituted alkyl, hydroxy-substituted alkyl, alkoxy-substituted alkyl, carboxy-substituted alkyl, amino-substituted alkyl, amido-substituted alkyl, carbamate-substituted alkyl, halogen-substituted alkyl, thio-substituted alkyl, azido-substituted alkyl, dithio-substituted alkyl, or sulfonyl-substituted alkyl;
further provided that when E is OCO, then J is not CO₂H, NH₂, or CH(CO₂R)₂;
further provided that when E is C═C, then J is not H or alkyl; and
further provided that when E is NH, then J is not CO₂R;
or a pharmaceutically acceptable salt thereof.

32. The compound of claim 31, wherein R⁶² is selected from the group consisting of Cl, Br, I, CH₃, CH₂CH₃, or CH═CH₂.

33. The compound of claim 32, wherein R⁶² is CH₂CH₃.

34. The compound of claim 33, wherein R⁶³ is CH₃.

35. The compound of claim 31, wherein R⁶⁴ is selected from the group consisting of CH₃, CH₂CH₃, phenyl, 3-ethyl-4-methylphenyl, 4-hydroxybutyl, 4-aminobutyl, and 4-methoxybutyl.

36. The compound of claim 31, wherein J is 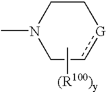
in which G is O, S, NR¹⁰¹, or C(R¹⁰²)(R¹⁰³), in which each of R¹⁰¹-R¹⁰³ is, independently, absent, H, C₁₋₆ alkyl, C₆₋₂₀ aryl, C₄₋₆ heteroaryl, C₇₋₁₂ arylalkyl, or COR¹⁰⁴, in which R¹⁰⁴ is substituted or unsubstituted C₆₋₁₀ aryl or substituted or unsubstituted C₄₋₆ heteroaryl, OR¹⁰⁵, in which R¹⁰⁵ is H, substituted or unsubstituted C₁₋₆ alkyl, substituted or unsubstituted C₁₋₆ cycloalkyl, substituted or unsubstituted C₆₋₁₂ aryl, substituted or unsubstituted C₇₋₂₀ arylalkyl, substituted or unsubstituted C₇₋₂₀ alkylaryl, substituted or unsubstituted C₄₋₁₀ heteroaryl, C₁₋₃ acyl, or C₁₋₆ sulfonyl;
each R¹⁰⁰ is, independently, substituted or unsubstituted C₁₋₆ alkyl, substituted or unsubstituted C₁₋₆ cycloalkyl, substituted or unsubstituted C₆₋₁₂ aryl, substituted or unsubstituted C₇₋₂₀ arylalkyl, substituted or unsubstituted C₇₋₂₀ alkylaryl, substituted or unsubstituted C₄₋₁₀ heteroaryl, C₁₋₃ acyl, or C₁₋₆ sulfonyl, OR¹⁰⁶, CH₂OR¹⁰⁸, in which each of R¹⁰⁶ and R¹⁰⁸ is, independently, H, substituted or unsubstituted C₁₋₆ alkyl, substituted or unsubstituted C₁₋₆ cycloalkyl, substituted or unsubstituted C₆₋₁₂ aryl, substituted or unsubstituted C₇₋₂₀ arylalkyl, substituted or unsubstituted C₇₋₂₀ alkylaryl, substituted or unsubstituted C₄₋₁₀ heteroaryl, C₁₋₃ acyl, or C₁₋₆ sulfonyl, or CO₂R¹¹⁰, where R¹¹⁰ is substituted or unsubstituted C₆₋₁₂ aryl, or substituted or unsubstituted C₄₋₆ heteroaryl;
and y is 0-2.

37. A pharmaceutical composition comprising a compound of claim 31 and a pharmaceutically acceptable carrier.

38. A method of inhibiting growth of Gram-positive bacteria in vitro, the method comprising contacting the bacteria with an effective amount of a compound of claim 31.

39. A method of inhibiting growth of Gram-positive bacteria, the method comprising contacting the bacteria with an effective amount of a compound of claim 31.

40. A method of treating a subject with a Gram-positive bacterial infection, the method comprising administering to the subject a therapeutically effective amount of a compound of claim 31.

41. A method of treating a subject with a mycoplasmal infection, the method comprising administering to the subject a therapeutically effective amount of a compound of claim 31.

42. A method of prophylactically treating a subject susceptible to a Gram-positive bacterial infection, the method comprising administering to the subject a therapeutically effective amount of a compound of claim 31.

43. A method of prophylactically treating a subject susceptible to a mycoplasmal infection, the method comprising administering to the subject a therapeutically effective amount of a compound of claim 31.
